(19) United States
(12) Patent Application Publication
(10) Pub. No.: US 2003/0203869 A1
(43) Pub. Date: Oct. 30, 2003

(54) Expression vectors for stimulating an immune response and methods of using the same

(76) Inventors: John D. Fikes, San Diego, CA (US); Gary G. Hermanson, Encinitas, CA (US); Alessandro Sette, La Jolla, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/371,525

(22) Filed: Feb. 21, 2003

Related U.S. Application Data
(62) Division of application No. 09/311,784, filed on May 13, 1999, now Pat. No. 6,534,482.
Application No. 09/311,784 is a continuation of application No. 09/078,904, filed on May 13, 1998, now abandoned.

(51) Int. Cl. A61K 48/00; C07H 21/04

(52) U.S. Cl. 514/44; 536/23.5

(57) ABSTRACT

The present invention relates to nucleic acid vaccines encoding multiple CTL and HTL epitopes and MHC targeting sequences.

### CROSS-REFERENCES TO RELATED APPLICATIONS

This patent application is a divisional of U.S. patent application Ser. No. 09/311,784, filed May 13, 1999, which is a continuation of U.S. patent application Ser. No. 09/078,904, filed May 13, 1998. This patent application also claims the benefit of U.S. Patent Application No. 60/085,751, filed May 15, 1998. Each of these documents is herein incorporated by reference in its entirety.

### STATEMENT AS TO RIGHTS TO INVENTIONS MADE UNDER FEDERALLY SPONSORED RESEARCH AND DEVELOPMENT

This invention was made with government support under NIH Grant No. AI-42699-01, NIH Grant No. AI38584-03, and NIH Contract No. N01-AI-45241. The Government has certain rights in this invention.

### FIELD OF THE INVENTION

The present invention relates to nucleic acid vaccines encoding multiple CTL and HTL epitopes and MHC targeting sequences.

### BACKGROUND OF THE INVENTION

Vaccines are of fundamental importance in modern medicine and have been highly effective in combating certain human diseases. However, despite the successful implementation of vaccination programs that have greatly limited or virtually eliminated several debilitating human diseases, there are a number of diseases that affect millions worldwide for which effective vaccines have not been developed.

Major advances in the field of immunology have led to a greater understanding of the mechanisms involved in the immune response and have provided insights into developing new vaccine strategies (Kuby, Immunology, 443-457 (3rd ed., 1997), which is incorporated herein by reference). These new vaccine strategies have taken advantage of knowledge gained regarding the mechanisms by which foreign material, termed antigen, is recognized by the immune system and eliminated from the organism. An effective vaccine is one that elicits an immune response to an antigen of interest.

Specialized cells of the immune system are responsible for the protective activity required to combat diseases. An immune response involves two major groups of cells, lymphocytes, or white blood cells, and antigen-presenting cells. The purpose of these immune response cells is to recognize foreign material, such as an infectious organism or a cancer cell, and remove that foreign material from the organism.

Two major types of lymphocytes mediate different aspects of the immune response. B cells display on their cell surface specialized proteins, called antibodies, that bind specifically to foreign material, called antigens. Effector B cells produce soluble forms of the antibody, late throughout the body and function to eliminate antigen from the organism. This branch of the immune system is known as the humoral branch. Memory B cells function to recognize the antigen in future encounters by continuing to express the membrane-bound form of the antibody.

A second major type of lymphocyte is the T cell. T cells also have on their cell surface specialized proteins that recognize antigen but, in contrast to B cells, require that the antigen be bound to a specialized membrane protein complex, the major histocompatibility complex (MHC), on the surface of an antigen-presenting cell. Two major classes of T cells, termed helper T lymphocytes (“HTL”) and cytotoxic T lymphocytes (“CTL”), are often distinguished based on the presence of either CD4 or CD8 protein, respectively, on the cell surface. This branch of the immune system is known as the cell-mediated branch.

The second major class of immune response cells are cells that function in antigen presentation by processing antigen for binding to MHC molecules expressed in the antigen presenting cells. The processed antigen bound to MHC molecules is transferred to the surface of the cell, where the antigen-MHC complex is available to bind to T cells.

MHC molecules can be divided into MHC class I and class II molecules and are recognized by the two classes of T cells. Nearly all cells express MHC class I molecules, which function to present antigen to cytotoxic T lymphocytes. Cytotoxic T lymphocytes typically recognize antigen bound to MHC class I. A subset of cells called antigen-presenting cells express MHC class II molecules. Helper T lymphocytes typically recognize antigen bound to MHC class II molecules. Antigen-presenting cells include dendritic cells, macrophages, B cells, fibroblasts, glial cells, pancreatic beta cells, thymic epithelial cells, thyroid epithelial cells and vascular endothelial cells. These antigen-presenting cells generally express both MHC class I and class II molecules. Also, B cells function as both antibody-producing and antigen-presenting cells.

Once a helper T lymphocyte recognizes an antigen-MHC class II complex on the surface of an antigen-presenting cell, the helper T lymphocyte becomes activated and produces growth factors that activate a variety of cells involved in the immune response, including B cells and cytotoxic T lymphocytes. For example, under the influence of growth factors expressed by activated helper T lymphocytes, a cytotoxic T lymphocyte that recognizes an antigen-MHC class I complex becomes activated. CTLs monitor and eliminate cells that display antigen specifically recognized by the CTL, such as infected cells or tumor cells. Thus, activation of helper T lymphocytes stimulates the activation of both the humoral and cell-mediated branches of the immune system.

An important aspect of the immune response, in particular as it relates to vaccine efficacy, is the manner in which antigen is processed so that it can be recognized by the specialized cells of the immune system. Distinct antigen processing and presentation pathways are utilized. The one is a cytosolic pathway, which results in the antigen being bound to MHC class I molecules. An alternative pathway is an endoplasmic reticulum pathway, which bypasses the cytosol. Another is an endocytic pathway, which results in the antigen being bound to MHC class II molecules. Thus, the cell surface presentation of a particular antigen by a MHC class II or class I molecule to a helper T lymphocyte or a cytotoxic T lymphocyte, respectively, is dependent on the processing pathway for that antigen.

The cytosolic pathway processes endogenous antigens that are expressed inside the cell. The antigen is degraded by a specialized protease complex in the cytosol of the cell, and the resulting antigen peptides are transported into the endoplasmic reticulum, an organelle that processes cell surface molecules. In the endoplasmic reticulum, the antigen peptides bind to MHC class I molecules, which are then transported to the cell surface for presentation to cytotoxic T lymphocytes of the immune system.

Antigens that exist outside the cell are processed by the endocytic pathway. Such antigens are taken into the cell by endocytosis, which brings the antigens into specialized vesicles called endosomes and subsequently to specialized vesicles called lysosomes, where the antigen is degraded by proteases into antigen peptides that bind to MHC class II molecules. The antigen peptide-MHC class II molecule complex is then transported to the cell surface for presentation to helper T lymphocytes of the immune system.

A variety of factors must be considered in the development of an effective vaccine. For example, the extent of activation of either the humoral or cell-mediated branch of the immune system can determine the effectiveness of a vaccine against a particular disease. Furthermore, the development of immunologic memory by inducing memory-cell formation can be important for an effective vaccine against a particular disease (Kuby, supra). For example, protection from infectious diseases caused by pathogens with short incubation periods, such as influenza virus, requires high levels of neutralizing antibody generated by the humoral branch because disease symptoms are already underway before memory cells are activated. Alternatively, protection from infectious diseases caused by pathogens with long incubation periods, such as polio virus, does not require neutralizing antibodies at the time of infection but instead requires memory B cells that can generate neutralizing antibodies to combat the pathogen before it is able to infect target tissues. Therefore, the effectiveness of a vaccine at preventing or ameliorating the symptoms of a particular disease depends on the type of immune response generated by the vaccine.

Many traditional vaccines have relied on intact pathogens such as attenuated or inactivated viruses or bacteria to elicit an immune response. However, these traditional vaccines have advantages and disadvantages, including reversion of an attenuated pathogen to a virulent form. The problem of reversion of an attenuated vaccine has been addressed by the use of molecules of the pathogen rather than the whole pathogen. For example, immunization approaches have begun to incorporate recombinant vector vaccines and synthetic peptide vaccines (Kuby, supra). Recently, DNA vaccines have also been used (Donnelly et al., Annu. Rev. Immunol. 15:617-648 (1997), which is incorporated herein by reference). The use of molecules of a pathogen provides safe vaccines that circumvent the potential for reversion to a virulent form of the vaccine.

The targeting of antigens to MHC class II molecules to activate helper T lymphocytes has been described using lysosomal targeting sequences, which direct antigens to lysosomes, where the antigen is digested by lysosomal proteases into antigen peptides that bind to MHC class II molecules (U.S. Pat. No. 5,633,234; Thomson et al., J. Virol. 72:2246-2252 (1998)). It would be advantageous to develop vaccines that deliver multiple antigens while exploiting the safety provided by administering individual epitopes of a pathogen rather than a whole organism. In particular, it would be advantageous to develop vaccines that effectively target antigens to MHC class II molecules for activation of helper T lymphocytes.

Several studies also point to the crucial role of cytotoxic T cells in both production and eradication of infectious diseases and cancer by the immune system (Byrne et al., J. Immunol. 51:682 (1984); McMichael et al., N. Engl. J. Med. 309:13 (1983)). Recombinant protein vaccines do not reliably induce CTL responses, and the use of otherwise immunogenic vaccines consisting of attenuated pathogens in humans is hampered, in the case of several important diseases, by overriding safety concerns. In the case of diseases such as HIV, HBV, HCV, and malaria, it appears desirable not only to induce a vigorous CTL response, but also to focus the response against highly conserved epitopes in order to prevent escape by mutation and overcome variable vaccine efficacy against different isolates of the target pathogen.

Induction of a broad response directed simultaneously against multiple epitopes also appears to be crucial for development of efficacious vaccines. HIV infection is perhaps the best example where an infected host may benefit from a multispecific response. Rapid progression of HIV infection has been reported in cases where a narrowly focused CTL response is induced whereas nonprogressors tend to show a broader specificity of CTLs (Goulder et al., Nat. Med. 3:212 (1997); Borrow et al., Nat. Med. 3:205 (1997)). The highly variable nature of HIV CTL epitopes resulting from a highly mutating genome and selection by CTL responses directed against only a single or few epitopes also supports the need for broad epitope CTL responses (McMichael et al., Annu. Rev. Immunol. 15:271 (1997)).

One potential approach to induce multispecific responses against conserved epitopes is immunization with a minigene plasmid encoding the epitopes in a string-of-beads fashion. Induction of CTL, HTL, and B cell responses in mice by minigene plasmids have been described by several laboratories using constructs encoding as many as 11 epitopes (An et al., J. Virol. 71:2292 (1997); Thomson et al., J. Immunol. 157:822 (1996); Whitton et al., J. Virol. 67:348 (1993); Hanke et al., Vaccine 16:426 (1998); Vitiello et al., Eur. J. Immunol. 27:671-678 (1997)). Minigenes have been delivered in vivo by infection with recombinant adenovirus or vaccinia, or by injection of purified DNA via the intramuscular or intradermal route (Thomson et al., J. Immunol. 160:1717 (1998); Toes et al., Proc. Natl. Acad. Sci. USA 94:14660 (1997)).

Successful development of minigene DNA vaccines for human use will require addressing certain fundamental questions dealing with epitope MHC affinity, optimization of constructs for maximum in vivo immunogenicity, and development of assays for testing in vivo potency of multi-epitope minigene constructs. Regarding MHC binding affinity of epitopes, it is not currently known whether both high and low affinity epitopes can be included within a single minigene construct, and what ranges of peptide affinity are permissible for CTL induction in vivo. This is especially important because dominant epitopes can vary in their affinity and because it might be important to be able to deliver mixtures of dominant and subdominant epitopes that are characterized by high and low MHC binding affinities.

With respect to minigene construct optimization for maximum immunogenicity in vivo, conflicting data exists regarding whether the exact position of the epitopes in a given construct or the presence of flanking regions, helper T cell epitopes, and signal sequences might be crucial for CTL induction (Del Val et al., Cell 66:1145 (1991); Bergmann et al., J. Virol. 68:5306 (1994); Thomson et al., Proc. Natl. Acad. Sci. USA 92:5845 (1995); Shirai et al., J. Infect. Dis. 173:24 (1996); Rahemtulla et al., Nature 353:180 (1991); Jennings et al., Cell. Immunol. 133:234 (1991); Anderson et al., J. Exp. Med. 174:489 (1991); Uger et al., J. Immunol. 158:685 (1997)). Finally, regarding development of assays that allow testing of human vaccine candidates, it should be noted that, to date, all in vivo immunogenicity data of multi-epitope minigene plasmids have been performed with murine class I MHC-restricted epitopes. It would be advantageous to be able to test the in vivo immunogenicity of minigenes containing human CTL epitopes in a convenient animal model system.

Thus, there exists a need to develop methods to effectively deliver a variety of HTL (helper T lymphocyte) and CTL (cytotoxic T lymphocyte) antigens to stimulate an immune response. The present invention satisfies this need and provides related advantages as well.

### SUMMARY OF THE INVENTION

The invention therefore provides expression vectors encoding two or more HTL epitopes fused to a MHC class II targeting sequence, as well as expression vectors encoding a CTL epitope and a universal HTL epitope fused to an MHC class I targeting sequence. The HTL epitope can be a universal HTL epitope (also referred to as a universal MHC class II epitope). The invention also provides expression vectors encoding two or more HTL epitopes fused to a MHC class II targeting sequence and encoding one or more CTL epitopes. The invention additionally provides methods of stimulating an immune response by administering an expression vector of the invention in vivo, as well as methods of assaying the human immunogenicity of a human T cell peptide epitope in vivo in a non-human mammal.

In one aspect, the present invention provides an expression vector comprising a promoter operably linked to a first nucleotide sequence encoding a major histocompatibility (MHC) targeting sequence fused to a second nucleotide sequence encoding two or more heterologous peptide epitopes, wherein the heterologous peptide epitopes comprise two HTL peptide epitopes or a CTL peptide epitope and a universal HTL peptide epitope.

In another aspect, the present invention provides a method of inducing an immune response in vivo comprising administering to a mammalian subject an expression vector comprising a promoter operably linked to a first nucleotide sequence encoding a major histocompatibility (MHC) targeting sequence fused to a second nucleotide sequence encoding two or more heterologous peptide epitopes, wherein the heterologous peptide epitopes comprise two HTL peptide epitopes or a CTL peptide epitope and a universal HTL peptide epitope.

In another aspect, the present invention provides a method of inducing an immune response in vivo comprising administering to a mammalian subject an expression vector comprising a promoter operably linked to a first nucleotide sequence encoding a major histocompatibility (MHC) targeting sequence fused to a second nucleotide sequence encoding a heterologous human HTL peptide epitope.

In another aspect, the present invention provides a method of assaying the human immunogenicity of a human T cell peptide epitope in vivo in a non-human mammal, comprising the step of administering to the non-human mammal an expression vector comprising a promoter operably linked to a first nucleotide sequence encoding a heterologous human CTL or HTL peptide epitope.

In one embodiment, the heterologous peptide epitopes comprise two or more heterologous HTL peptide epitopes. In another embodiment, the heterologous peptide epitopes comprise a CTL peptide epitope and a universal HTL peptide epitope. In another embodiment, the heterologous peptide epitopes further comprise one to two or more heterologous CTL peptide epitopes. In another embodiment, the expression vector comprises both HTL and CTL peptide epitopes.

In one embodiment, one of the HTL peptide epitopes is a universal HTL epitope. In another embodiment, the universal HTL epitope is a pan DR epitope. In another embodiment, the pan DR epitope has the sequence AlaLysPheValAlaAlaTrpThrLeuLysAlaAlaAla (SEQ ID NO:52).

In one embodiment, the peptide epitopes are hepatitis B virus epitopes, hepatitis C virus epitopes, human immunodeficiency virus epitopes, human papilloma virus epitopes, MAGE epitopes, PSA epitopes, PSM epitopes, PAP epitopes, p53 epitopes, CEA epitopes, Her2/neu epitopes, or Plasmodium epitopes. In another embodiment, the peptide epitopes each have a sequence selected from the group consisting of the peptides depicted in Tables 1-8. In another embodiment, at least one of the peptide epitopes is an analog of a peptide depicted in Tables 1-8.

In one embodiment, the MHC targeting sequence comprises a region of a polypeptide selected from the group consisting of the li protein, LAMP-I, HLS-DM, HLA-DO, H2-DO, influenza matrix protein, hepatitis B surface antigen, hepatitis B virus core antigen, Ty particle, Ig-α protein, Ig-β protein, and Ig kappa chain signal sequence.

In one embodiment, the expression vector further comprises a second promoter sequence operably linked to a third nucleotide sequence encoding one or more heterologous HTL or CTL peptide epitopes. In another embodiment, the CTL peptide epitope comprises a structural motif for an HLA supertype, whereby the peptide CTL epitope binds to two or more members of the supertype with an affinity of greater that 500 nM. In another embodiment, the CTL peptide epitopes have structural motifs that provide binding affinity for more than one HLA allele supertype.

In one embodiment, the non-human mammal is a transgenic mouse that expresses a human HLA allele. In another embodiment, the human HLA allele is selected from the group consisting of A11 and A2.1. In another embodiment, the non-human mammal is a macaque that expresses a human HLA allele.

### BRIEF DESCRIPTION OF THE DRAWINGS

FIG. 1 shows the nucleotide and amino acid sequences (SEQ ID NOS:1 and 2, respectively) of the IiPADRE construct encoding a fusion of the murine Ii gene with a pan DR epitope sequence substituted for the CLIP sequence of the Ii protein.

FIG. 2 shows the nucleotide and amino acid sequences (SEQ ID NOS:3 and 4, respectively) of the 180T construct encoding a fusion of the cytoplasmic domain, the transmembrane domain and part of the luminal domain of the Ii protein fused to multiple MHC class II epitopes.

FIG. 3 shows the nucleotide and amino acid sequences (SEQ ID NOS:5 and 6, respectively) of the IiThfull construct encoding a fusion of the cytoplasmic domain, transmembrane domain and a portion of the luminal domain of the Ii protein fused to multiple T helper epitopes and amino acid residues 101 to 215 of the Ii protein, which encodes the trimerization region of the Ii protein.

FIG. 4 shows the nucleotide and amino acid sequences (SEQ ID NOS:7 and 8, respectively) of the KappaLAMP-Th construct encoding a fusion of the murine immunoglobulin kappa signal sequence fused to multiple T helper epitopes and the transmembrane and cytoplasmic domains of LAMP-1.

FIG. 5 shows the nucleotide and amino acid sequences (SEQ ID NOS:9 and 10, respectively) of the H2M-Th construct encoding a fusion of the signal sequence of H2-M fused to multiple MHC class II epitopes and the transmembrane and cytoplasmic domains of H2-M.

FIG. 6 shows the nucleotide and amino acid sequences (SEQ ID NOS:11 and 12, respectively) of the H₂O-Th construct encoding a fusion of the signal sequence of H2-DO fused to multiple MHC class II epitopes and the transmembrane and cytoplasmic domains of H2-DO.

FIG. 7 shows the nucleotide and amino acid sequences (SEQ ID NOS:13 and 14, respectively) of the PADRE-Influenza matrix construct encoding a fusion of a pan DR epitope sequence fused to the amino-terminus of influenza matrix protein.

FIG. 8 shows the nucleotide and amino acid sequences (SEQ ID NOS:15 and 16, respectively) of the PADRE-HBV-s construct encoding a fusion of a pan DR epitope sequence fused to the amino-terminus of hepatitis B virus surface antigen.

FIG. 9 shows the nucleotide and amino acid sequences (SEQ ID NOS:17 and 18, respectively) of the Ig-alphaTh construct encoding a fusion of the signal sequence of the Ig-α protein fused to multiple MHC class II epitopes and the transmembrane and cytoplasmic the Ig-α protein.

FIG. 10 shows the nucleotide and amino acid sequences (SEQ ID NOS:19 and 20, respectively) of the Ig-betaTh construct encoding a fusion of the signal sequence of the Ig-β protein fused to multiple MHC class II epitopes and the transmembrane and cytoplasmic domains of the Ig-β protein.

FIG. 11 shows the nucleotide and amino acid sequences (SEQ ID NOS:21 and 22, respectively) of the SigTh construct encoding a fusion of the signal sequence of the kappa immunoglobulin fused to multiple MHC class II epitopes.

FIG. 12 shows the nucleotide and amino acid sequences (SEQ ID NOS:23 and 24, respectively) of human HLA-DR, the invariant chain (Ii) protein.

FIG. 13 shows the nucleotide and amino acid sequences (SEQ ID NOS:25 and 26, respectively) of human lysosomal membrane glycoprotein-1 (LAMP-1).

FIG. 14 shows the nucleotide and amino acid sequences (SEQ ID NOS:27 and 28, respectively) of human HLA-DMB.

FIG. 15 shows the nucleotide and amino acid sequences (SEQ ID NOS:29 and 30, respectively) of human HLA-DO beta.

FIG. 16 shows the nucleotide and amino acid sequences (SEQ ID NOS:31 and 32, respectively) of the human MB-I Ig-α.

FIG. 17 shows the nucleotide and amino acid sequences (SEQ ID NOS:33 and 34, respectively) of human Ig-β protein.

FIG. 18 shows a schematic diagram depicting the method of generating some of the encoding a MHC class II targeting sequence fused to multiple MHC class II epitopes.

FIG. 19 shows the nucleotide sequence of the vector pEP2 (SEQ ID NO:35).

FIG. 20 shows the nucleotide and amino acid sequences of the vector pMIN.0 (SEQ ID NOS:36 and 37, respectively).

FIG. 21 shows the nucleotide and amino acid sequences of the vector pMIN.1 (SEQ ID NOS:38 and 39, respectively).

FIG. 22. Representative CTL responses in HLA-A2.1/K^{b}-H-2^{bxs} mice immunized with pMin.1 DNA. Splenocytes from primed animals were cultured in triplicate flasks and stimulated twice in vitro with each peptide epitope. Cytotoxicity of each culture was assayed in a ⁵¹Cr release assay against Jurkat-A2.1/K^{b} target cells in the presence (filled symbols, solid lines) or absence (open symbols, dotted lines) of peptide. Each symbol represents the response of a single culture.

FIG. 23. Presentation of viral epitopes to specific CTLs by Jurkat-A2.1/K^{b} tumor cells transfected with DNA minigene. Two constructs were used for transfection, pMin.1 and pMin.2-GFP. pMin.2-GFP-transfected targets cells were sorted by FACS and the population used in this experiment contained 60% fluorescent cells. CTL stimulation was measured by quantitating the amount of IFN-γ release (A, B) or by lysis of ⁵¹Cr-labeled target cells (C, D, hatched bars). CTLs were stimulated with transfected cells (A, C) or with parental Jurkat-A2.1/K^{b} cells in the presence of 1 μg/ml peptide (B, D). Levels of IFN-γ release and cytotoxicity for the different CTL lines in the absence of epitope ranged from 72-126 pg/ml and 2-6% respectively.

FIG. 24. Summary of modified minigene constructs used to address variables critical for in vivo immunogenicity. The following modifications were incorporated into the prototype pMin.1 construct; A, deletion of PADRE HTL epitope; B, incorporation of the native HBV Pol 551 epitope that contains an alanine in position 9; C, deletion of the Ig kappa signal sequence; and D, switching position of the HBV Env 335 and HBV Pol 455 epitopes.

FIG. 25. Examination of variables that may influence pMin.1 immunogenicity. In vivo CTL-inducing activity of pMin.1 is compared to modified constructs. For ease of comparison, the CTL response induced by each of the modified DNA minigene constructs (shaded bars) is compared separately in each of the four panels to the response induced by the prototype pMin.1 construct (solid bars). The geometric mean response of CTL-positive cultures from two to five independent experiments are shown. Numbers shown with each bar indicate the number of positive cultures/total number tested for that particular epitope. The ratio of positive cultures/total tested for the pMin.1 group is shown in panel A and is the same for the remaining Figure panels (see Example V, Materials and Methods, in vitro CTL cultures, for the definition of a positive CTL culture). Theradigm responses were obtained by immunizing animals with the lipopeptide and stimulating and testing splenocyte cultures with the HBV Core 18-27 peptide.

Definitions

An “HTL” peptide epitope or an “MHC II epitope” is an MHC class II restricted epitope, i.e., one that is bound by an MHC class II molecule.

A “CTL” peptide epitope or an “MHC I epitope” is an MHC class I restricted epitope, i.e., one that is bound by an MHC class I molecule.

An “MHC targeting sequence” refers to a peptide sequence that targets a polypeptide, e.g., comprising a peptide epitope, to a cytosolic pathway (e.g., an MHC class I antigen processing pathway), en endoplasmic reticulum pathways, or an endocytic pathway (e.g., an MHC class II antigen processing pathway).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature, e.g., a fusion polypeptide comprising subsequence from different polypeptides, peptide epitopes from the same polypeptide that are not naturally in an adjacent position, or repeats of a single peptide epitope.

As used herein, the term “universal MHC class II epitope” or a “universal HTL epitope” refers to a MHC class II peptide epitope that binds to gene products of multiple MHC class II alleles. For example, the DR, DP and DQ alleles are human MHC II alleles. Generally, a unique set of peptides binds to a particular gene product of a MHC class II allele. In contrast, a universal MHC class II epitope is able to bind to gene products of multiple MHC class II alleles. A universal MHC class II epitope binds to 2 or more MHC class II alleles, generally 3 or more MHC class II alleles, and particularly 5 or more MHC class II alleles. Thus, the presence of a universal MHC class II epitope in an expression vector is advantageous in that it function to increase the number of allelic MHC class II molecules that can bind to the peptide and, consequently, the number of Helper T lymphocytes that are activated.

Universal MHC class II epitopes are well known in the art and include, for example, epitopes such as the “pan DR epitopes,” also referred to as “PADRE” (Alexander et al., Immunity 1:751-761 (1994); WO 95/07707, U.S. S No. 60/036,713, U.S. S No. 60/037,432, PCT/US98/01373, U.S. Ser. No. 09/009,953, and U.S. S No. 60/087,192 each of which is incorporated herein by reference). A “pan DR binding peptide” or a “PADRE” peptide of the invention is a peptide capable of binding at least about 7 different DR molecules, preferably 7 of the 12 most common DR molecules, most preferably 9 of the 12 most common DR molecules (DR1, 2w2b, 2w2a, 3, 4w4, 4w14, 5, 7, 52a, 52b, 52c, and 53), or alternatively, 50% of a panel of DR molecules representative of greater than or equal to 75% of the human population, preferably greater than or equal to 80% of the human population. Pan DR epitopes can bind to a number of DR alleles and are strongly immunogenic for T cells. For example, pan DR epitopes were found to be more effective at inducing an immune response than natural MHC class II epitopes (Alexander, supra). An example of a PADRE epitope is the peptide AlaLysPheValAlaAlaTrpThrLeuLysAlaAlaAla (SEQ ID NO:52).

With regard to a particular amino acid sequence, an “epitope” is a set of amino acid residues which is involved in recognition by a particular immunoglobulin, or in the context of T cells, those residues necessary for recognition by T cell receptor proteins and/or Major Histocompatibility Complex (MHC) receptors. In an immune system setting, in vivo or in vitro, an epitope is the collective features of a molecule, such as primary, secondary and tertiary peptide structure, and charge, that together form a site recognized by an immunoglobulin, T cell receptor or HLA molecule. Throughout this disclosure epitope and peptide are often used interchangeably. It is to be appreciated, however, that isolated or purified protein or peptide molecules larger than and comprising an epitope of the invention are still within the bounds of the invention.

As used herein, “high affinity” with respect to HLA class I molecules is defined as binding with an IC50 (or KD) of less than 50 nM. “Intermediate affinity” is binding with an IC50 (or KD) of between about 50 and about 500 nM. “High affinity” with respect to binding to HLA class II molecules is defined as binding with an KD of less than 100 nM. “Intermediate affinity” is binding with a KD of between about 100 and about 1000 nM. Assays for determining binding are described in detail, e.g., in PCT publications WO 94/20127 and WO 94/03205. Alternatively, binding is expressed relative to a reference peptide. As a particular assay becomes more, or less, sensitive, the IC50s of the peptides tested may change somewhat. However, the binding relative to the reference peptide will not significantly change. For example, in an assay run under conditions such that the IC50 of the reference peptide increases 10-fold, the IC50 values of the test peptides will also shift approximately 10-fold. Therefore, to avoid ambiguities, the assessment of whether a peptide is a good, intermediate, weak, or negative binder is generally based on its IC50, relative to the IC50 of a standard peptide.

Throughout this disclosure, results are expressed in terms of “IC50s.” IC50 is the concentration of peptide in a binding assay at which 50% inhibition of binding of a reference peptide is observed. Given the conditions in which the assays are run (i.e., limiting HLA proteins and labeled peptide concentrations), these values approximate KD values. It should be noted that IC50 values can change, often dramatically, if the assay conditions are varied, and depending on the particular reagents used (e.g., HLA preparation, etc.). For example, excessive concentrations of HLA molecules will increase the apparent measured IC50 of a given ligand.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithms using default program parameters or by manual alignment and visual inspection.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Major histocompatibility complex” or “MHC” is a cluster of genes that plays a role in control of the cellular interactions responsible for physiologic immune responses. In humans, the MHC complex is also known as the HLA complex. For a detailed description of the MHC and HLA complexes, see Paul, Fundamental Immunology (3rd ed. 1993).

“Human leukocyte antigen” or “HLA” is a human class I or class II major histocompatibility complex (MHC) protein (see, e.g., Stites, et al., Immunology, (8th Ed., 1994).

An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, HLA family, and HLA xx-like supertype molecules (where xx denotes a particular HLA type), are synonyms.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “supermotif” is a peptide binding specificity shared by HLA molecules encoded by two or more HLA alleles. Thus, a preferably is recognized with high or intermediate affinity (as defined herein) by two or more HLA antigens.

“Cross-reactive binding” indicates that a peptide is bound by more than one HLA molecule; a synonym is degenerate binding.

The term “peptide” is used interchangeably with “oligopeptide” in the present specification to designate a series of residues, typically L-amino acids, connected one to the other, typically by peptide bonds between the a-amino and carboxyl groups of adjacent amino acids. The preferred CTL-inducing oligopeptides of the invention are 13 residues or less in length and usually consist of between about 8 and about 11 residues, preferably 9 or 10 residues. The preferred HTL-inducing oligopeptides are less than about 50 residues in length and usually consist of between about 6 and about 30 residues, more usually between about 12 and 25, and often between about 15 and 20 residues.

An “immunogenic peptide” or “peptide epitope” is a peptide which comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

A “protective immune response” refers to a CTL and/or an HTL response to an antigen derived from an infectious agent or a tumor antigen, which prevents or at least partially arrests disease symptoms or progression. The immune response may also include an antibody response which has been facilitated by the stimulation of helper T cells.

The term “residue” refers to an amino acid or amino acid mimetic incorporated into an oligopeptide by an amide bond or amide bond mimetic.

“Synthetic peptide” refers to a peptide that is not naturally occurring, but is man-made using such methods as chemical synthesis or recombinant DNA technology.

The nomenclature used to describe peptide compounds follows the conventional practice wherein the amino group is presented to the left (the N-terminus) and the carboxyl group to the right (the C-terminus) of each amino acid residue. When amino acid residue positions are referred to in a peptide epitope they are numbered in an amino to carboxyl direction with position one being the position closest to the amino terminal end of the epitope, or the peptide or protein of which it may be a part. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G.

As used herein, the term “expression vector” is intended to refer to a nucleic acid molecule capable of expressing an antigen of interest such as a MHC class I or class II epitope in an appropriate target cell. An expression vector can be, for example, a plasmid or virus, including DNA or RNA viruses. The expression vector contains such a promoter element to express an antigen of interest in the appropriate cell or tissue in order to stimulate a desired immune response.

### DETAILED DESCRIPTION OF THE INVENTION

Cytotoxic T lymphocytes (CTLs) and helper T lymphocytes (HTLs) are critical for immunity against infectious pathogens; such as viruses, bacteria, and protozoa; tumor cells; autoimmune diseases and the like. The present invention provides minigenes that encode peptide epitopes which induce a CTL and/or HTL response. The minigenes of the invention also include an MHC targeting sequence. A variety of minigenes encoding different epitopes can be tested for immunogenicity using an HLA transgenic mouse. The epitopes are typically a combination of at least two or more HTL epitopes, or a CTL epitope plus a universal HTL epitope, and optimally include additional HTI and/or CTL epitopes. Two, three, four, five, six, seven, eight, nine, ten, twenty, thirty, forty or about fifty different epitopes, either HTL and/or CTL, can be included in the minigene, along with the MHC targeting sequence. The epitopes can have different HLA restriction. Epitopes to be tested include those derived from viruses such as HIV, HBV, HCV, HSV, CMV, HPV, and HTLV; cancer antigens such as p53, Her2/Neu, MAGE, PSA, human papilloma virus, and CEA; parasites such as Trypanosoma, Plasmodium, Leishmania, Giardia, Entamoeba; autoimmune diseases such as rheumatoid arthritis, myesthenia gravis, and lupus erythematosus; fungi such as Aspergillus and Candida; and bacteria such as Escherichia coli, Staphylococci, Chlamydia, Mycobacteria, Streptococci, and Pseudomonas. The epitopes to be encoded by the minigene are selected and tested using the methods described in published PCT applications WO 93/07421, WO 94/02353, WO 95/01000, WO 97/04451, and WO 97/05348, herein incorporated by reference.

HTL and CTL Epitopes

The expression vectors of the invention encode one or more MHC class II and/or class I epitopes and an MHC targeting sequence. Multiple MHC class II or class I epitopes present in an expression vector can be derived from the same antigen, or the MHC epitopes can be derived from different antigens. For example, an expression vector can contain one or more MHC epitopes that can be derived from two different antigens of the same virus or from two different antigens of different viruses. Furthermore, any MHC epitope can be used in the expression vectors of the invention. For example, any single MHC epitope or a combination of the MHC epitopes shown in Tables 1 to 8 can be used in the expression vectors of the invention. Other peptide epitopes can be selected by one of skill in the art, e.g., by using a computer to select epitopes that contain HLA allele-specific motifs or supermotifs. The expression vectors of the invention can also encode one or more universal MHC class II epitopes, e.g., PADRE (see, e.g., SEQ ID NO:52).

Universal MHC class II epitopes can be advantageously combined with other MHC class I and class II epitopes to increase the number of cells that are activated in response to a given antigen and provide broader population coverage of MHC-reactive alleles. Thus, the expression vectors of the invention can encode MHC epitopes specific for an antigen, universal MHC class II epitopes, or a combination of specific MHC epitopes and at least one universal MHC class II epitope.

MHC class I epitopes are generally about 5 to 15 amino acids in length, in particular about 8 to 11 amino acids in length. MHC class II epitopes are generally about 10 to 25 amino acids in length, in particular about 13 to 21 amino acids in length. A MHC class I or II epitope can be derived from any desired antigen of interest. The antigen of interest can be a viral antigen, surface receptor, tumor antigen, oncogene, enzyme, or any pathogen, cell or molecule for which an immune response is desired. Epitopes can be selected based on their ability to bind one or multiple HLA alleles, and can also be selected using the “analog” technique described below.

Targeting Sequences

The expression vectors of the invention encode one or more MHC epitopes operably linked to a MHC targeting sequence. The use of a MHC targeting sequence enhances the immune response to an antigen, relative to delivery of antigen alone, by directing the peptide epitope to the site of MHC molecule assembly and transport to the cell surface, thereby providing an increased number of MHC molecule-peptide epitope complexes available for binding to and activation of T cells.

MHC class I targeting sequences are used in the present invention, e.g., those sequences that target an MHC class I epitope peptide to a cytosolic pathway or to the endoplasmic reticulum (see, e.g., Rammensee et al., Immunogenetics 41:178-228 (1995)). For example, the cytosolic pathway processes endogenous antigens that are expressed inside the cell. Although not wishing to be bound by any particular theory, cytosolic proteins are thought to be at least partially degraded by an endopeptidase activity of a proteasome and then transported to the endoplasmic reticulum by the TAP molecule (transporter associated with processing). In the endoplasmic reticulum, the antigen binds to MHC class I molecules. Endoplasmic reticulum signal sequences bypass the cytosolic processing pathway and directly target endogenous antigens to the endoplasmic reticulum, where proteolytic degradation into peptide fragments occurs. Such MHC class I targeting sequences are well known in the art, and include, e.g., signal sequences such as those from Ig kappa tissue plasminogen activator or insulin. A preferred signal peptide is the human Ig kappa chain sequence. Endoplasmic reticulum signal sequences can also be used to target MHC class II epitopes to the endoplasmic reticulum, the site of MHC class I molecule assembly.

MHC class II targeting sequences are also used in the invention, e.g., those that target a peptide to the endocytic pathway. These targeting sequences typically direct extracellular antigens to enter the endocytic pathway, which results in the antigen being transferred to the lysosomal compartment where the antigen is proteolytically cleaved into antigen peptides for binding to MHC class II molecules. As with the normal processing of exogenous antigen, a sequence that directs a MHC class II epitope to the endosomes of the endocytic pathway and/or subsequently to lysosomes, where the MHC class II epitope can bind to a MHC class II molecule, is a MHC class II targeting sequence. For example, group of MHC class II targeting sequences useful in the invention are lysosomal targeting sequences, which localize polypeptides to lysosomes. Since MHC class II molecules typically bind to antigen peptides derived from proteolytic processing of endocytosed antigens in lysosomes, a lysosomal targeting sequence can function as a MHC class II targeting sequence. Lysosomal targeting sequences are well known in the art and include sequences found in the lysosomal proteins LAMP-1 and LAMP-2 as described by August et al. (U.S. Pat. No. 5,633,234, issued May 27, 1997), which is incorporated herein by reference.

Other lysosomal proteins that contain lysosomal targeting sequences include HLA-DM. HLA-DM is an endosomal/lysosomal protein that functions in facilitating binding of antigen peptides to MHC class II molecules. Since it is located in the lysosome, HLA-DM has a lysosomal targeting sequence that can function as a MHC class II molecule targeting sequence (Copier et al., J. Immunol. 157:1017-1027 (1996), which is incorporated herein by reference).

The resident lysosomal protein HLA-DO can also function as a lysosomal targeting sequence. In contrast to the above described resident lysosomal proteins LAMP-1 and HLA-DM, which encode specific Tyr-containing motifs that target proteins to lysosomes, HLA-DO is targeted to lysosomes by association with HLA-DM (Liljedahl et al., EMBO J. 15:4817-4824 (1996)), which is incorporated herein by reference. Therefore, the sequences of HLA-DO that cause association with HLA-DM and, consequently, translocation of HLA-DO to lysosomes can be used as MHC class II targeting sequences. Similarly, the murine homolog of HLA-DO, H2-DO, can be used to derive a MHC class II targeting sequence. A MHC class II epitope can be fused to HLA-DO or H2-DO and targeted to lysosomes.

In another example, the cytoplasmic domains of B cell receptor subunits Ig-α and Ig-β mediate antigen internalization and increase the efficiency of antigen presentation (Bonnerot et al., Immunity 3:335-347 (1995)), which is incorporated herein by reference. Therefore, the cytoplasmic domains of the Ig-α and Ig-β proteins can function as MHC class II targeting sequences that target a MHC class II epitope to the endocytic pathway for processing and binding to MHC class II molecules.

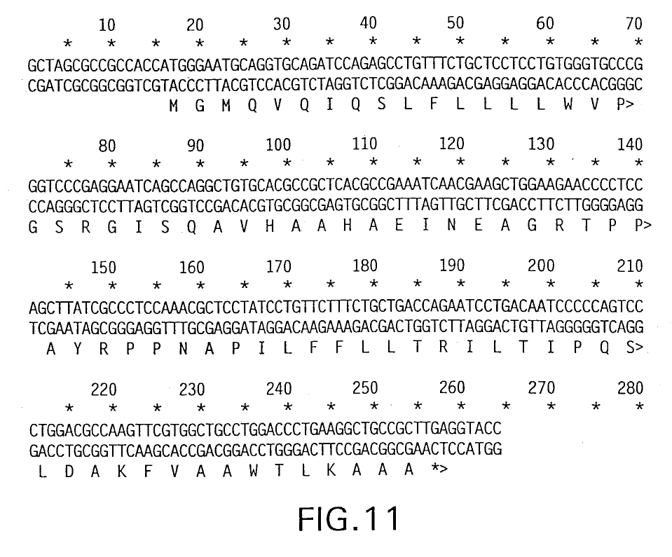

Another example of a MHC class II targeting sequence that directs MHC class II epitopes to the endocytic pathway is a sequence that directs polypeptides to be secreted, where the polypeptide can enter the endosomal pathway. These MHC class II targeting sequences that direct polypeptides to be secreted mimic the normal pathway by which exogenous, extracellular antigens are processed into peptides that bind to MHC class II molecules. Any signal sequence that functions to direct a polypeptide through the endoplasmic reticulum and ultimately to be secreted can function as a MHC class II targeting sequence so long as the secreted polypeptide can enter the endosomal/lysosomal pathway and be cleaved into peptides that can bind to MHC class II molecules. An example of such a fusion is shown in FIG. 11, where the signal sequence of kappa immunoglobulin is fused to multiple MHC class II epitopes.

In another example, the Ii protein binds to MHC class II molecules in the endoplasmic reticulum, where it functions to prevent peptides present in the endoplasmic reticulum from binding to the MHC class II molecules. Therefore, fusion of a MHC class II epitope to the Ii protein targets the MHC class II epitope to the endoplasmic reticulum and a MHC class II molecule. For example, the CLIP sequence of the Ii protein can be removed and replaced with a MHC class II epitope sequence so that the MHC class II epitope is directed to the endoplasmic reticulum, where the epitope binds to a MHC class II molecule.

In some cases, antigens themselves can serve as MHC class II or I targeting sequences and can be fused to a universal MHC class II epitope to stimulate an immune response. Although cytoplasmic viral antigens are generally processed and presented as complexes with MHC class I molecules, long-lived cytoplasmic proteins such as the influenza matrix protein can enter the MHC class II molecule processing pathway (Gueguen & Long, Proc. Natl. Acad. Sci. USA 93:14692-14697 (1996)), which is incorporated herein by reference. Therefore, long-lived cytoplasmic proteins can function as a MHC class II targeting sequence. For example, an expression vector encoding influenza matrix protein fused to a universal MHC class II epitope can be advantageously used to target influenza antigen and the universal MHC class II epitope to the MHC class II pathway for stimulating an immune response to influenza.

Other examples of antigens functioning as MHC class II targeting sequences include polypeptides that spontaneously form particles. The polypeptides are secreted from the cell that produces them and spontaneously form particles, which are taken up into an antigen-presenting cell by endocytosis such as receptor-mediated endocytosis or are engulfed by phagocytosis. The particles are proteolytically cleaved into antigen peptides after entering the endosomal/lysosomal pathway.

One such polypeptide that spontaneously forms particles is HBV surface antigen (HBV-S) (Diminsky et al., Vaccine 15:637-647 (1997); Le Borgne et al., Virology 240:304-315 (1998)), each of which is incorporated herein by reference. Another polypeptide that spontaneously forms particles is HBV core antigen (Kuhrober et al., International Immunol. 9:1203-1212 (1997)), which is incorporated herein by reference. Still another polypeptide that spontaneously forms particles is the yeast Ty protein (Weber et al., Vaccine 13:831-834 (1995)), which is incorporated herein by reference. For example, an expression vector containing HBV-S antigen fused to a universal MHC class II epitope can be advantageously used to target HBV-S antigen and the universal MHC class II epitope to the MHC class II pathway for stimulating an immune response to HBV.

Binding Affinity of Peptide Epitopes for HLA Molecules

The large degree of HLA polymorphism is an important factor to be taken into account with the epitope-based approach to vaccine development. To address this factor, epitope selection encompassing identification of peptides capable of binding at high or intermediate affinity to multiple HLA molecules is preferably utilized, most preferably these epitopes bind at high or intermediate affinity to two or more allele specific HLA molecules.

CTL-inducing peptides of interest for vaccine compositions preferably include those that have a binding affinity for class I HLA molecules of less than 500 nM. HTL-inducing peptides preferably include those that have a binding affinity for class II HLA molecules of less than 1000 nM. For example, peptide binding is assessed by testing the capacity of a candidate peptide to bind to a purified HLA molecule in vitro. Peptides exhibiting high or intermediate affinity are then considered for further analysis. Selected peptides are tested on other members of the supertype family. In preferred embodiments, peptides that exhibit cross-reactive binding are then used in vaccines or in cellular screening analyses.

Higher HLA binding affinity is typically correlated with greater immunogenicity. Greater immunogenicity can be manifested in several different ways. Immunogenicity corresponds to whether an immune response is elicited at all, and to the vigor of any particular response, as well as to the extent of a population in which a response is elicited. For example, a peptide might elicit an immune response in a diverse array of the population, yet in no instance produce a vigorous response. In accordance with these principles, close to 90% of high binding peptides have been found to be immunogenic, as contrasted with about 50% of the peptides which bind with intermediate affinity. Moreover, higher binding affinity peptides leads to more vigorous immunogenic responses. As a result, less peptide is required to elicit a similar biological effect if a high affinity binding peptide is used. Thus, in preferred embodiments of the invention, high binding epitopes are particularly useful.

The relationship between binding affinity for HLA class I molecules and immunogenicity of discrete peptide epitopes on bound antigens has been determined for the first time in the art by the present inventors. The correlation between binding affinity and immunogenicity was analyzed in two different experimental approaches (Sette et al., J. Immunol. 153:5586-5592 (1994)). In the first approach, the immunogenicity of potential epitopes ranging in HLA binding affinity over a 10,000-fold range was analyzed in HLA-A*0201 transgenic mice. In the second approach, the antigenicity of approximately 100 different hepatitis B virus (HBV)-derived potential epitopes, all carrying A*0201 binding motifs, was assessed by using PBL (peripheral blood lymphocytes) from acute hepatitis patients. Pursuant to these approaches, it was determined that an affinity threshold of approximately 500 nM (preferably 50 nM or less) determines the capacity of a peptide epitope to elicit a CTL response. These data are true for class I binding affinity measurements for naturally processed peptides and for synthesized T cell epitopes. These data also indicate the important role of determinant selection in the shaping of T cell responses (see, e.g., Schaeffer et al. Proc. Natl. Acad. Sci. USA 86:4649-4653, 1989).

An affinity threshold associated with immunogenicity in the context of HLA class II DR molecules has also been delineated (see, e.g., Southwood et al. J. Immunology 160:3363-3373 (1998), and U.S. S No. 60/087,192, filed May 29, 1998). In order to define a biologically significant threshold of DR binding affinity, a database of the binding affinities of 32 DR-restricted epitopes for their restricting element (i.e., the HLA molecule that binds the motif) was compiled. In approximately half of the cases (15 of 32 epitopes), DR restriction was associated with high binding affinities, i.e. binding affinities of less-than 100 nM. In the other half of the cases (16 of 32), DR restriction was associated with intermediate affinity (binding affinities in the 100-1000 nM range). In only one of 32 cases was DR restriction associated with an IC50 of 1000 nM or greater. Thus, 1000 nM can be defined as an affinity threshold associated with immunogenicity in the context of DR molecules.

Peptide Epitope Binding Motifs and Supermotifs

In the past few years evidence has accumulated to demonstrate that a large fraction of HLA class I and class II molecules can be classified into a relatively few supertypes, each characterized by largely overlapping peptide binding repertoires, and consensus structures of the main peptide binding pockets.

For HLA molecule pocket analyses, the residues comprising the B and F pockets of HLA class I molecules as described in crystallographic studies were analyzed (Guo et al., Nature 360:364 (1992); Saper et al., J. Mol. Biol. 219:277 (1991); Madden et al., Cell 75:693 (1993); Parham et al., Immunol. Rev. 143:141 (1995)). In these analyses, residues 9, 45, 63, 66, 67, 70, and 99 were considered to make up the B pocket; and the B pocket was deemed to determine the specificity for the amino acid residue in the second position of peptide ligands. Similarly, residues 77, 80, 81, and 116 were considered to determine the specificity of the F pocket; the F pocket was deemed to determine the specificity for the C-terminal residue of a peptide ligand bound by the HLA class I molecule.

Through the study of single amino acid substituted antigen analogs and the sequencing of endogenously bound, naturally processed peptides, critical residues required for allele-specific binding to HLA molecules have been identified. The presence of these residues correlates with binding affinity for HLA molecules. The identification of motifs and/or supermotifs that correlate with high and intermediate affinity binding is an important issue with respect to the identification of immunogenic peptide epitopes for the inclusion in a vaccine. Kast et al. (J. Immunol. 152:3904-3912 (1994)) have shown that motif-bearing peptides account for 90% of the epitopes that bind to allele-specific HLA class I molecules. In this study all possible peptides of 9 amino acids in length and overlapping by eight amino acids (240 peptides), which cover the entire sequence of the E6 and E7 proteins of human papillomavirus type 16, were evaluated for binding to five allele-specific HLA molecules that are expressed at high frequency among different ethnic groups. This unbiased set of peptides allowed an evaluation of the predictive value of HLA class I motifs. From the set of 240 peptides, 22 peptides were identified that bound to an allele-specific HLA molecules with high or intermediate affinity. Of these 22 peptides, 20, (i.e., 91%), were motif-bearing. Thus, this study demonstrates the value of motifs for the identification of peptide epitopes for inclusion in a vaccine: application of motif-based identification techniques eliminates screening of 90% of the potential epitopes in a target antigen protein sequence.

Peptides of the present invention may also include epitopes that bind to MHC class II DR molecules. There is a significant difference between class I and class II HLA molecules. This difference corresponds to the fact that, although a stringent size restriction and motif position relative to the binding pocket exists for peptides that bind to class I molecules, a greater degree of heterogeneity in both size and binding frame position of the motif, relative to the N and C termini of the peptide, exists for class II peptide ligands.

This increased heterogeneity of HLA class II peptide ligands is due to the structure of the binding groove of the HLA class II molecule which, unlike its class I counterpart, is open at both ends. Crystallographic analysis of HLA class II DRB*0101-peptide complexes showed that the residues occupying position 1 and position 6 of peptides complexed with DRB*0101 engage two complementary pockets on the DRBa*0101 molecules, with the P1 position corresponding to the most crucial anchor residue and the deepest hydrophobic pocket (see, e.g., Madden, Ann. Rev. Immunol. 13:587 (1995)). Other studies have also pointed to the P6 position as a crucial anchor residue for binding to various other DR molecules.

Thus, peptides of the present invention are identified by any one of several HLA class I or II-specific amino acid motifs (see, e.g., Tables I-III of U.S. Ser. Nos. 09/226,775, and 09/239,043, herein incorporated by reference in their entirety). If the presence of the motif corresponds to the ability to bind several allele-specific HLA antigens it is referred to as a supermotif. The allele-specific HLA molecules that bind to peptides that possess a particular amino acid supermotif are collectively referred to as an HLA “supertype.”

Immune Response-Stimulating Peptide Analogs

In general, CTL and HTL responses are not directed against all possible epitopes. Rather, they are restricted to a few “immunodominant” determinants (Zinkemagel et al., Adv. Immunol. 27:5159 (1979); Bennink et al., J. Exp. Med. 168:1935-1939 (1988); Rawle et al., J. Immunol. 146:3977-3984 (1991)). It has been recognized that immunodominance (Benacerraf et al., Science 175:273-279 (1972)) could be explained by either the ability of a given epitope to selectively bind a particular HLA protein (determinant selection theory) (Vitiello et al., J. Immunol. 131:1635 (1983)); Rosenthal et al., Nature 267:156-158 (1977)), or being selectively recognized by the existing TCR (T cell receptor) specificity (repertoire theory) (Klein, Immunology, The Science of Self on self Discrimination, pp. 270-310 (1982)). It has been demonstrated that additional factors, mostly linked to processing events, can also play a key role in dictating, beyond strict immunogenicity, which of the many potential determinants will be presented as immunodominant (Sercarz et al., Annu. Rev. Immunol. 11:729-766 (1993)).

The concept of dominance and subdominance is relevant to immunotherapy of both infectious diseases and cancer. For example, in the course of chronic viral disease, recruitment of subdominant epitopes can be important for successful clearance of the infection, especially if dominant CTL or HTL specificities have been inactivated by functional tolerance, suppression, mutation of viruses and other mechanisms (Franco et al., Curr. Opin. Immunol. 7:524-531 (1995)). In the case of cancer and tumor antigens, CTLs recognizing at least some of the highest binding affinity peptides might be functionally inactivated. Lower binding affinity peptides are preferentially recognized at these times, and may therefore be preferred in therapeutic or prophylactic anti-cancer vaccines.

In particular, it has been noted that a significant number of epitopes derived from known non-viral tumor associated antigens (TAA) bind HLA class I with intermediate affinity (IC50 in the 50-500 nM range). For example, it has been found that 8 of 15 known TAA peptides recognized by tumor infiltrating lymphocytes (TIL) or CTL bound in the 50-500 nM range. (These data are in contrast with estimates that 90% of known viral antigens were bound by HLA class I molecules with IC50 of 50 nM or less, while only approximately 10% bound in the 50-500 nM range (Sette et al., J. Immunol., 153:558-5592 (1994)). In the cancer setting this phenomenon is probably due to elimination, or functional inhibition of the CTL recognizing several of the highest binding peptides, presumably because of T cell tolerization events.

Without intending to be bound by theory, it is believed that because T cells to dominant epitopes may have been clonally deleted, selecting subdominant epitopes may allow extant T cells to be recruited, which will then lead to a therapeutic or prophylactic response. However, the binding of HLA molecules to subdominant epitopes is often less vigorous than to dominant ones. Accordingly, there is a need to be able to modulate the binding affinity of particular immunogenic epitopes for one or more HLA molecules, and thereby to modulate the immune response elicited by the peptide, for example to prepare analog peptides which elicit a more vigorous response. This ability would greatly enhance the usefulness of peptide-based vaccines and therapeutic agents.

Thus, although peptides with suitable cross-reactivity among all alleles of a superfamily are identified by the screening procedures described above, cross-reactivity is not always as complete as possible, and in certain cases procedures to further increase cross-reactivity of peptides can be useful; moreover, such procedures can also be used to modify other properties of the peptides such as binding affinity or peptide stability. Having established the general rules that govern cross-reactivity of peptides for HLA alleles within a given motif or supermotif, modification (i.e., analoging) of the structure of peptides of particular interest in order to achieve broader (or otherwise modified) HLA binding capacity can be performed. More specifically, peptides which exhibit the broadest cross-reactivity patterns, can be produced in accordance with the teachings herein. The present concepts related to analog generation are set forth in greater detail in co-pending U.S. Ser. No. 09/226,775.

In brief, the strategy employed utilizes the motifs or supermotifs which correlate with binding to certain HLA class I and II molecules. The motifs or supermotifs are defined by having primary anchors, and in many cases secondary anchors (see Tables I-III of U.S. Ser. No. 09/226,775). Analog peptides can be created by substituting amino acids residues at primary anchor, secondary anchor, or at primary and secondary anchor positions. Generally, analogs are made for peptides that already bear a motif or supermotif. Preferred secondary anchor residues of supermotifs and motifs that have been defined for HLA class I and class II binding peptides are shown in Tables II and III, respectively, of U.S. Ser. No. 09/226,775.

For a number of the motifs or supermotifs in accordance with the invention, residues are defined which are deleterious to binding to allele-specific HLA molecules or members of HLA supertypes that bind to the respective motif or supermotif (see Tables II and III of U.S. Ser. No. 09/226,775). Accordingly, removal of such residues that are detrimental to binding can be performed in accordance with the methods described therein. For example, in the case of the A3 supertype, when all peptides that have such deleterious residues are removed from the population of analyzed peptides, the incidence of cross-reactivity increases from 22% to 37% (I., Sidney et al., Hu. Immunol. 45:79 (1996)). Thus, one strategy to improve the cross-reactivity of peptides within a given supermotif is simply to delete one or more of the deleterious residues present within a peptide and substitute a small “neutral” residue such as Ala (that may not influence T cell recognition of the peptide). An enhanced likelihood of cross-reactivity is expected if, together with elimination of detrimental residues within a peptide, “preferred” residues associated with high affinity binding to an allele-specific HLA molecule or to multiple HLA molecules within a superfamily are inserted.

To ensure that an analog peptide, when used as a vaccine, actually elicits a CTL response to the native epitope in vivo (or, in the case of class II epitopes, a failure to elicit helper T cells that cross-react with the wild type peptides), the analog peptide may be used to immunize T cells in vitro from individuals of the appropriate HLA allele. Thereafter, the immunized cells' capacity to induce lysis of wild type peptide sensitized target cells is evaluated. In both class I and class II systems it will be desirable to use as targets, cells that have been either infected or transfected with the appropriate genes to establish whether endogenously produced antigen is also recognized by the relevant T cells.

Another embodiment of the invention is to create analogs of weak binding peptides, to thereby ensure adequate numbers of cross-reactive cellular binders. Class I peptides exhibiting binding affinities of 500-50000 nM, and carrying an acceptable but suboptimal primary anchor residue at one or both positions can be “fixed” by substituting preferred anchor residues in accordance with the respective supertype. The analog peptides can then be tested for crossbinding activity.

Another embodiment for generating effective peptide analogs involves the substitution of residues that have an adverse impact on peptide stability or solubility in, e.g., a liquid environment. This substitution may occur at any position of the peptide epitope. For example, a cysteine (C) can be substituted out in favor of gamma-amino butyric acid. Due to its chemical nature, cysteine has the propensity to form disulfide bridges and sufficiently alter the peptide structurally so as to reduce binding capacity. Substituting gamma-amino butyric acid for C not only alleviates this problem, but actually improves binding and crossbinding capability in certain instances (Sette et al, In: Persistent Viral Infections (Ahmed & Chen, eds., 1998)). Substitution of cysteine with gamma-amino butyric acid may occur at any residue of a peptide epitope, i.e., at either anchor or non-anchor positions.

Expression Vectors and Construction of a Minigene

The expression vectors of the invention contain at least one promoter element that is capable of expressing a transcription unit encoding the antigen of interest, for example, a MHC class I epitope or a MHC class II epitope and an MHC targeting sequence in the appropriate cells of an organism so that the antigen is expressed and targeted to the appropriate MHC molecule. For example, if the expression vector is administered to a mammal such as a human, a promoter element that functions in a human cell is incorporated into the expression vector. An example of an expression vector useful for expressing the MHC class II epitopes fused to MHC class II targeting sequences and the MHC class I epitopes described herein is the pEP2 vector described in Example IV.

This invention relies on routine techniques in the field of recombinant genetics. Basic texts disclosing the general methods of use in this invention include Sambrook et al, Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994); Oligonucleotide Synthesis: A Practical Approach (Gait, ed., 1984); Kuijpers, Nucleic Acids Research 18(17):5197 (1994); Dueholm, J. Org. Chem. 59:5767-5773 (1994); Methods in Molecular Biology, volume 20 (Agrawal, ed.); and Tijssen, Laboratory Techniques in Biochemistry and Molecular Biology—Hybridization with Nucleic Acid Probes, e.g., Part I, chapter 2 “Overview of principles of hybridization and the strategy of nucleic acid probe assays” (1993)).

The minigenes are comprised of two or many different epitopes (see, e.g., Tables 1-8). The nucleic acid encoding the epitopes are assembled in a minigene according to standard techniques. In general, the nucleic acid sequences encoding minigene epitopes are isolated using amplification techniques with oligonucleotide primers, or are chemically synthesized. Recombinant cloning techniques can also be used when appropriate. Oligonucleotide sequences are selected which either amplify (when using PCR to assemble the minigene) or encode (when using synthetic oligonucleotides to assemble the minigene) the desired epitopes.

Amplification techniques using primers are typically used to amplify and isolate sequences encoding the epitopes of choice from DNA or RNA (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify epitope nucleic acid sequences directly from mRNA, from cDNA, from genomic libraries or cDNA libraries. Restriction endonuclease sites can be incorporated into the primers. Minigenes amplified by the PCR reaction can be purified from agarose gels and cloned into an appropriate vector.

Synthetic oligonucleotides can also be used to construct minigenes. This method is performed using a series of overlapping oligonucleotides, representing both the sense and non-sense strands of the gene. These DNA fragments are then annealed, ligated and cloned. Oligonucleotides that are not commercially available can be chemically synthesized according to the solid phase phosphoramidite trimester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The epitopes of the minigene are typically subcloned into an expression vector that contains a strong promoter to direct transcription, as well as other regulatory sequences such as enhancers and polyadenylation sites. Suitable promoters are well known in the art and described, e.g., in Sambrook et al and Ausubel et al. Eukaryotic expression systems for mammalian cells are well known in the art and are commercially available. Such promoter elements include, for example, cytomegalovirus (CMV), Rous sarcoma virus LTR and SV40.

The expression vector typically contains a transcription unit or expression cassette that contains all the additional elements required for the expression of the minigene in host cells. A typical expression cassette thus contains a promoter operably linked to the minigene and signals required for efficient polyadenylation of the transcript. Additional elements of the cassette may include enhancers and introns with functional splice donor and acceptor sites.

In addition to a promoter sequence, the expression cassette can also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic cells may be used. Expression vectors containing regulatory elements from eukaryotic viruses are typically used in eukaryotic expression vectors, e.g., SV40 vectors, papilloma virus vectors, and vectors derived from Epstein Bar virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells. In one embodiment, the vector pEP2 is used in the present invention.

Other elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Administration In Vivo

The invention also provides methods for stimulating an immune response by administering an expression vector of the invention to an individual. Administration of an expression vector of the invention for stimulating an immune response is advantageous because the expression vectors of the invention target MHC epitopes to MHC molecules, thus increasing the number of CTL and HTL activated by the antigens encoded by the expression vector.

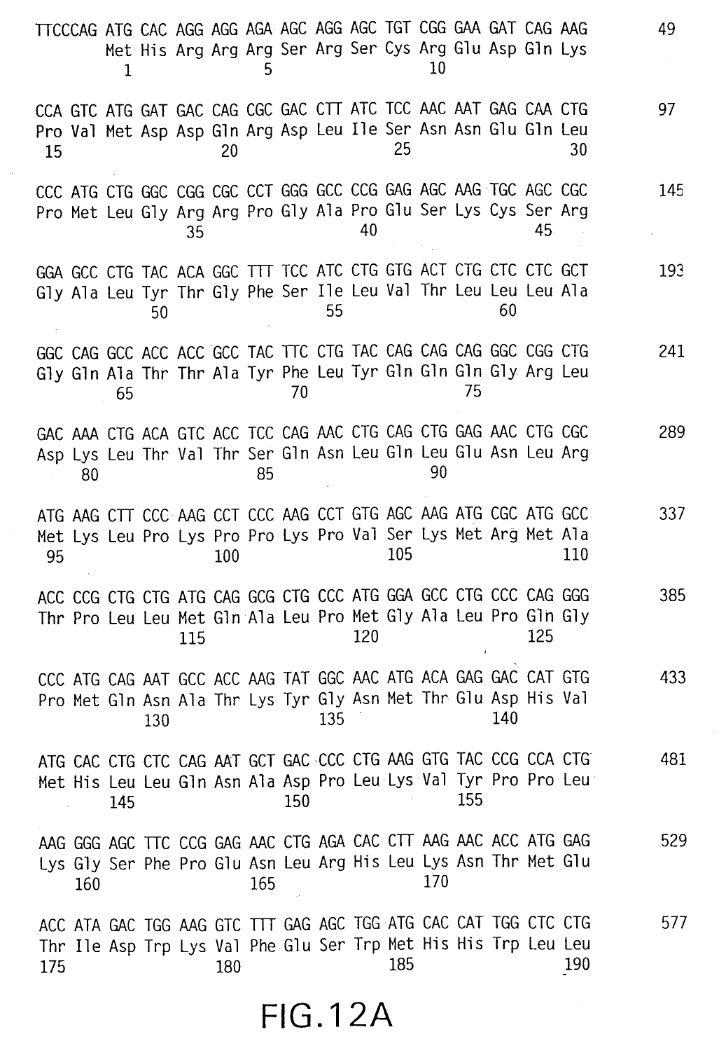

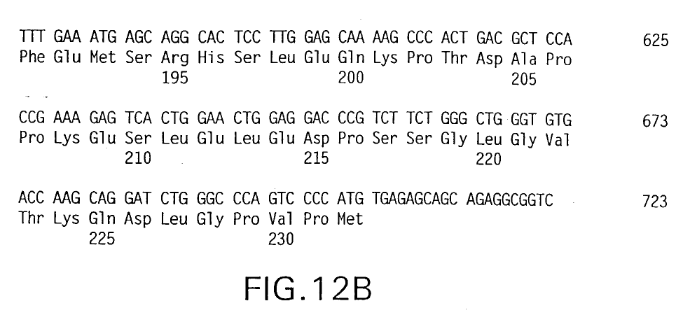

Initially, the expression vectors of the invention are screened in mouse to determine the expression vectors having optimal activity in stimulating a desired immune response. Initial studies are therefore carried out, where possible, with mouse genes of the MHC targeting sequences. Methods of determining the activity of the expression vectors of the invention are well known in the art and include, for example, the uptake of ³H-thymidine to measure T cell activation and the release of ⁵¹Cr to measure CTL activity as described below in Examples II and III. Experiments similar to those described in Example IV are performed to determine the expression vectors having activity at stimulating an immune response. The expression vectors having activity are further tested in human. To circumvent potential adverse immunological responses to encoded mouse sequences, the expression vectors having activity are modified so that the MHC class II targeting sequences are derived from human genes. For example, substitution of the analogous regions of the human homologs of genes containing various MHC class II targeting sequences are substituted into the expression vectors of the invention. Examples of such human homologs of genes containing MHC class II targeting sequences are shown in FIGS. 12 to 17. Expression vectors containing human MHC class II targeting sequences, such as those described in Example I below, are tested for activity at stimulating an immune response in human.

The invention also relates to pharmaceutical compositions comprising a pharmaceutically acceptable carrier and an expression vector of the invention. Pharmaceutically acceptable carriers are well known in the art and include aqueous or non-aqueous solutions, suspensions and emulsions, including physiologically buffered saline, alcohol/aqueous solutions or other solvents or vehicles such as glycols, glycerol, oils such as olive oil or injectable organic esters.

A pharmaceutically acceptable carrier can contain physiologically acceptable compounds that act, for example, to stabilize the expression vector or increase the absorption of the expression vector. Such physiologically acceptable compounds include, for example, carbohydrates, such as glucose, sucrose or dextrans, antioxidants such as ascorbic acid or glutathione, chelating agents, low molecular weight polypeptides, antimicrobial agents, inert gases or other stabilizers or excipients. Expression vectors can additionally be complexed with other components such as peptides, polypeptides and carbohydrates. Expression vectors can also be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The invention further relates to methods of administering a pharmaceutical composition comprising an expression vector of the invention to stimulate an immune response. The expression vectors are administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), each of which is incorporated herein by reference. In one embodiment, the minigene is administered as naked nucleic acid.

A pharmaceutical composition comprising an expression vector of the invention can be administered to stimulate an immune response in a subject by various routes including, for example, orally, intravaginally, rectally, or parenterally, such as intravenously, intramuscularly, subcutaneously, intraorbitally, intracapsularly, intraperitoneally, intracisternally or by passive or facilitated absorption through the skin using, for example, a skin patch or transdermal iontophoresis, respectively. Furthermore, the composition can be administered by injection, intubation or topically, the latter of which can be passive, for example, by direct application of an ointment or powder, or active, for example, using a nasal spray or inhalant. An expression vector also can be administered as a topical spray, in which case one component of the composition is an appropriate propellant. The pharmaceutical composition also can be incorporated, if desired, into liposomes, microspheres or other polymer matrices (Felgner et al., U.S. Pat. No. 5,703,055; Gregoriadis, Liposome Technology, Vols. I to III (2nd ed. 1993), each of which is incorporated herein by reference). Liposomes, for example, which consist of phospholipids or other lipids, are nontoxic, physiologically acceptable and metabolizable carriers that are relatively simple to make and administer.

The expression vectors of the invention can be delivered to the interstitial spaces of tissues of an animal body (Felgner et al., U.S. Pat. Nos. 5,580,859 and 5,703,055). Administration of expression vectors of the invention to muscle is a particularly effective method of administration, including intradermal and subcutaneous injections and transdermal administration. Transdermal administration, such as by iontophoresis, is also an effective method to deliver expression vectors of the invention to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647).

Other effective methods of administering an expression vector of the invention to stimulate an immune response include mucosal administration (Carson et al., U.S. Pat. No. 5,679,647). For mucosal administration, the most effective method of administration includes intranasal administration of an appropriate aerosol containing the expression vector and a pharmaceutical composition. Suppositories and topical preparations are also effective for delivery of expression vectors to mucosal tissues of genital, vaginal and ocular sites. Additionally, expression vectors can be complexed to particles and administered by a vaccine gun.

The dosage to be administered is dependent on the method of administration and will generally be between about 0.1 μg up to about 200 μg. For example, the dosage can be from about 0.05 μg/kg to about 50 mg/kg, in particular about 0.005-5 mg/kg. An effective dose can be determined, for example, by measuring the immune response after administration of an expression vector. For example, the production of antibodies specific for the MHC class II epitopes or MHC class I epitopes encoded by the expression vector can be measured by methods well known in the art, including ELISA or other immunological assays. In addition, the activation of T helper cells or a CTL response can be measured by methods well known in the art including, for example, the uptake of ³H-thymidine to measure T cell activation and the release of ⁵¹ Cr to measure CTL activity (see Examples II and III below).

The pharmaceutical compositions comprising an expression vector of the invention can be administered to mammals, particularly humans, for prophylactic or therapeutic purposes. Examples of diseases that can be treated or prevented using the expression vectors of the invention include infection with HBV, HCV, HIV and CMV as well as prostate cancer, renal carcinoma, cervical carcinoma, lymphoma, condyloma acuminatum and acquired immunodeficiency syndrome (AIDS).

In therapeutic applications, the expression vectors of the invention are administered to an individual already suffering from cancer, autoimmune disease or infected with a virus. Those in the incubation phase or acute phase of the disease can be treated with expression vectors of the invention, including those expressing all universal MHC class II epitopes, separately or in conjunction with other treatments, as appropriate.

In therapeutic and prophylactic applications, pharmaceutical compositions comprising expression vectors of the invention are administered to a patient in an amount sufficient to elicit an effective immune response to an antigen and to ameliorate the signs or symptoms of a disease. The amount of expression vector to administer that is sufficient to ameliorate the signs or symptoms of a disease is termed a therapeutically effective dose. The amount of expression vector sufficient to achieve a therapeutically effective dose will depend on the pharmaceutical composition comprising an expression vector of the invention, the manner of administration, the state and severity of the disease being treated, the weight and general state of health of the patient and the judgment of the prescribing physician.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following example is provided by way of illustration only and not by way of limitation. Those of skill in the art will readily recognize a variety of noncritical parameters that could be changed or modified to yield essentially similar results.

### Example I

### Construction of Expression Vectors Containing MHC Class II Epitopes

This example shows construction of expression vectors containing MHC class II epitopes that can be used to target antigens to MHC class II molecules.

Expression vectors comprising DNA constructs were prepared using overlapping oligonucleotides, polymerase chain reaction (PCR) and standard molecular biology techniques (Dieffenbach & Dveksler, PCR Primer: A Laboratory Manual (1995); Sambrook et al., Molecular Cloning: A Laboratory Manual (2nd ed., 1989), each of which is incorporated herein by reference).

To generate full length wild type Ii, the full length invariant chain was amplified, cloned, and sequenced and used in the construction of the three invariant chain constructs. Except where noted, the source of cDNA for all the constructs listed below was Mouse Spleen Marathon-Ready cDNA made from Balb/c males (Clontech; Palo Alto Calif.). The primer pairs were the oligonucleotide GCTAGCGCCGCCACCATGGATGACCAACGCGACCTC (SEQ ID NO:40), which is designated murli-F and contains an NheI site followed by the consensus Kozak sequence and the 5′ end of the Ii cDNA; and the oligonucleotide GGTACCTCACAGGGTGACTTGACCCAG (SEQ ID NO:41), which is designated murli-R and contains a KpnI site and the 3′ end of the Ii coding sequence.

For the PCR reaction, 5 μl of spleen cDNA and 250 nM of each primer were combined in a 100 μl reaction with 0.25 mM each dNTP and 2.5 units of Pfu polymerase in Pfu polymerase buffer containing 10 mM KCl, 10 mM (NH₄)₂SO₄, 20 mM Tris-chloride, pH 8.75, 2 mM MgSO₄, 0.1% TRITON X-100 and 100 μg/ml bovine serum albumin (BSA). A Perkin/Elmer 9600 PCR machine (Perkin Elmer; Foster City Calif.) was used and the cycling conditions were: 1 cycle of 95° C. for 5 minutes, followed by 30 cycles of 95° C. for 15 seconds, 52° C. for 30 seconds, and 72° C. for 1 minute. The PCR reaction was run on a 1% agarose gel, and the 670 base pair product was cut out, purified by spinning through a Millipore Ultrafree-MC filter (Millipore; Bedford Mass.) and cloned into pCR-Blunt from Invitrogen (San Diego, Calif.). Individual clones were screened by sequencing, and a correct clone (named bli#3) was used as a template for the helper constructs.

DNA constructs containing pan DR epitope sequences and MHC II targeting sequences derived from the Ii protein were prepared. The Ii murine protein has been previously described (Zhu & Jones, Nucleic Acids Res. 17:447-448 (1989)), which is incorporated herein by reference. Briefly, the IiPADRE construct contains the full length Ii sequence with PADRE precisely replacing the CLIP region. The DNA construct encodes amino acids 1 through 87 of invariant chain, followed with the 13 amino acid PADRE sequence (SEQ ID NO:52) and the rest of the invariant chain DNA sequence (amino acids 101-215). The construct was amplified in 2 overlapping halves that were joined to produce the final construct. The two primers used to amplify the 5′ half were murli-F and the oligonucleotide CAGGGTCCAGGCAGCCACGAACTTGGCCACAGGTTTGGCAGA (SEQ ID NO:42), which is designated IiPADRE-R. The IiPADRE-R primer includes nucleotides 303-262 of IiPADRE. The 3′ half was amplified with the primer GGCTGCCTGGACCCTGAAGGCTGCCGCTATGTCCATGGATAAC (SEQ ID NO:43), which is designated IiPADRE-F and includes nucleotides 288-330 of IiPADRE; and murli-R. The PCR conditions were the same as described above, and the two halves were isolated by agarose gel electrophoresis as described above.

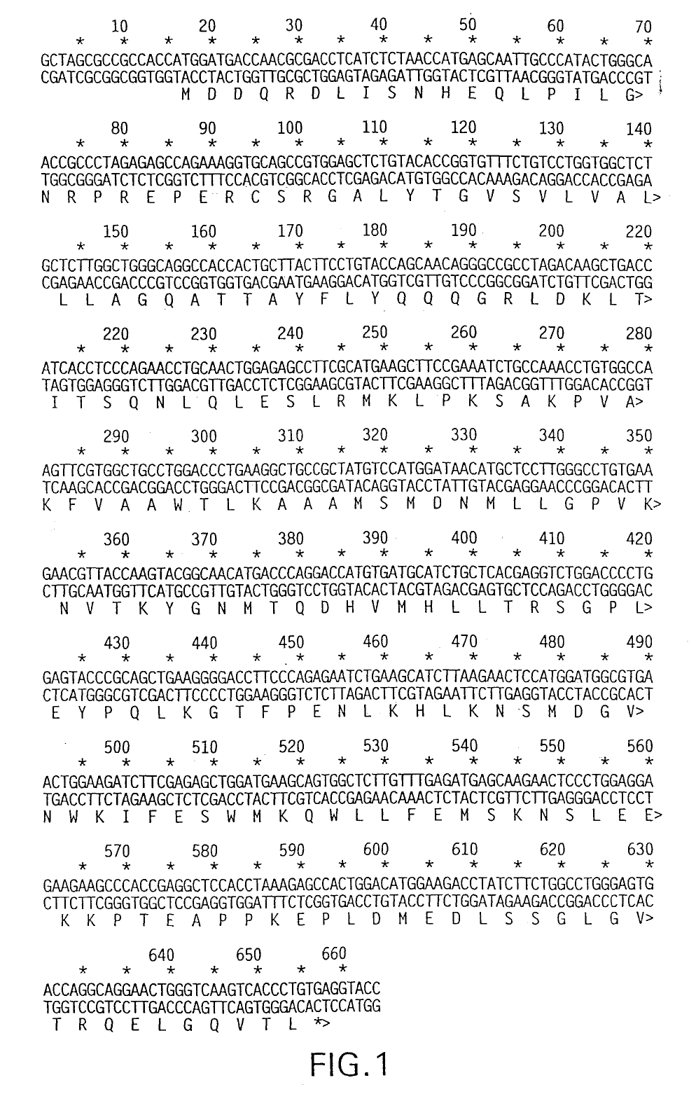

Ten microliters of each PCR product was combined in a 100 μl PCR reaction with an annealing temperature of 50° C. for five cycles to generate a full length template. Primers murli-F and murli-R were added and 25 more cycles carried out. The full length IiPADRE product was isolated, cloned, and sequenced as described above. This construct contains the murine Ii gene with a pan DR epitope sequence substituted for the CLIP sequence of Ii (FIG. 1).

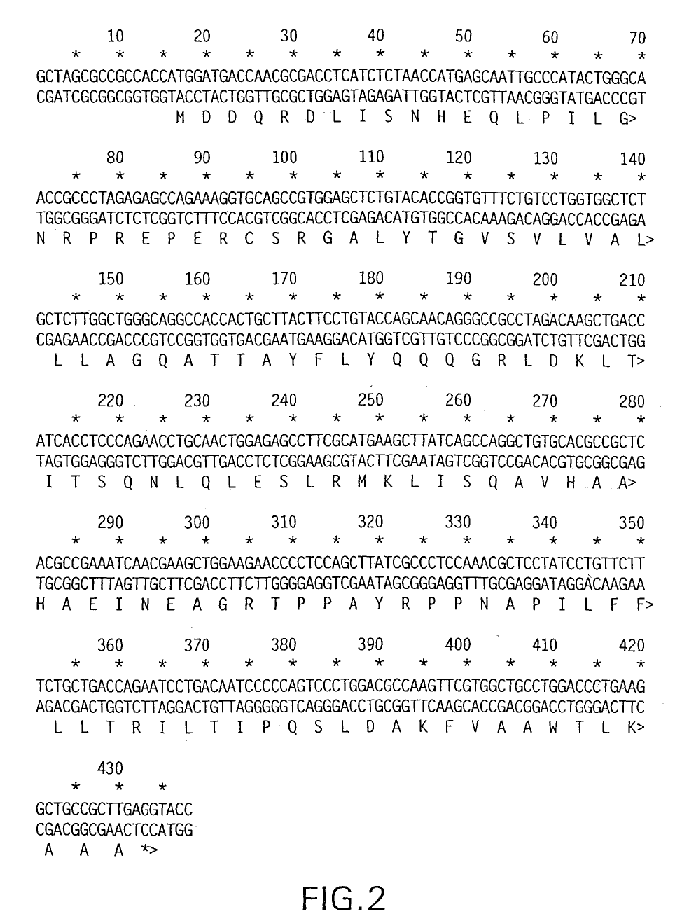

A DNA construct, designated 180T, containing the cytoplasmic domain, the transmembrane domain and part of the luminal domain of Ii fused to a string of multiple MHC class II epitopes was constructed (FIG. 2). Briefly, the string of multiple MHC class II epitopes was constructed with three overlapping oligonucleotides (oligos). Each oligo overlapped its neighbor by 15 nucleotides and the final MHC class II epitope string was assembled by extending the overlapping oligonucleotides in three sets of reactions using PCR. The three oligonucleotides were: oligo 1, nucleotides 241-310, CTTCGCATGAAGCTTATCAGCCAGGCTGTGCACGCCGCTCACGCCGAAATCAACGA AGCTGGAAGAACCC (SEQ ID NO:44); oligo 2, nucleotides 364-295, TTCTGGTCAGCAGAAAGAACAGGATAGGAGCGTTTGGAGGGCGATAAGCTGGAGGG GTTCTTCCAGCTTC (SEQ ID NO:45); and oligo 3, nucleotides 350-42, TTCTGCTGACCAGAATCCTGACAATCCCCCAGTCCCTGGACGCCAAGTTCGTGGCTG CCTGGACCCTGAAG (SEQ ID NO:46).

For the first PCR reaction, 5 μg of oligos 1 and 2 were combined in a 100 μl reaction containing Pfu polymerase. A Perkin/Elmer 9600 PCR machine was used and the annealing temperature used was 45° C. The PCR product was gel-purified, and a second reaction containing the PCR product of oligos 1 and 2 with oligo 3 was annealed and extended for 10 cycles before gel purification of the full length product to be used as a “mega-primer.”

The 180T construct was made by amplifying bli#3 with murli-F and the mega-primer. The cycling conditions were: 1 cycle of 95° C. for 5 minutes, followed by 5 cycles of 95° C. for 15 seconds, 37° C. for 30 seconds, and 72° C. for 1 minute. Primer Help-epR was added and an additional 25 cycles were carried out with the annealing temperature raised to 47° C. The Help-epR primer GGTACCTCAAGCGGCAGCCTTCAGGGTCCAGGCA (SEQ ID NO:47) corresponds to nucleotides 438-405. The full length 180T product was isolated, cloned, and sequenced as above.

The 180T construct (FIG. 2) encodes amino acid residues 1 through 80 of Ii, containing the cytoplasmic domain, the transmembrane domain and part of the luminal domain, fused to a string of multiple MHC class II epitopes corresponding to: amino acid residues 323-339 of ovalbumin (IleSerGlnAlaValHisAlaAlaHisAlaGluIleAsnGluAlaGlyArg; SEQ ID NO:48); amino acid residues 128 to 141 of HBV core antigen (amino acids ThrProProAlaTyrArgProProAsnAlaProIleLeu; SEQ ID NO:49); amino acid residues 182 to 196 of HBV env (amino acids PhePheLeuLeuThrArgIleLeuThrIleProGlnSerLeuAsp; SEQ ID NO:50); and the pan DR sequence designated SEQ ID NO:52.

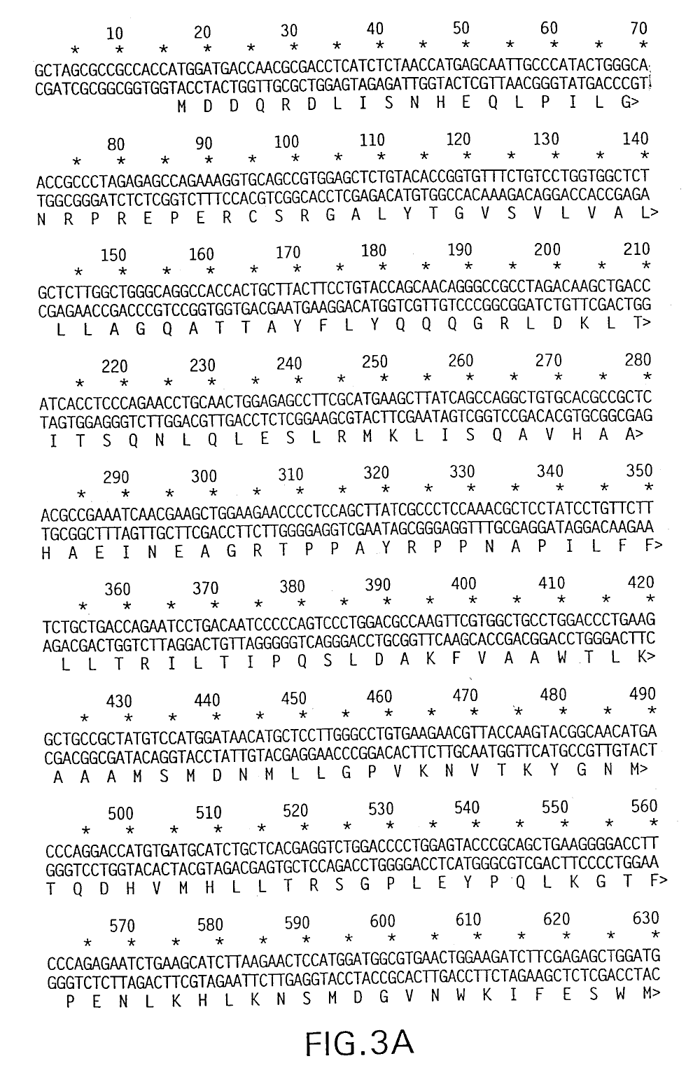

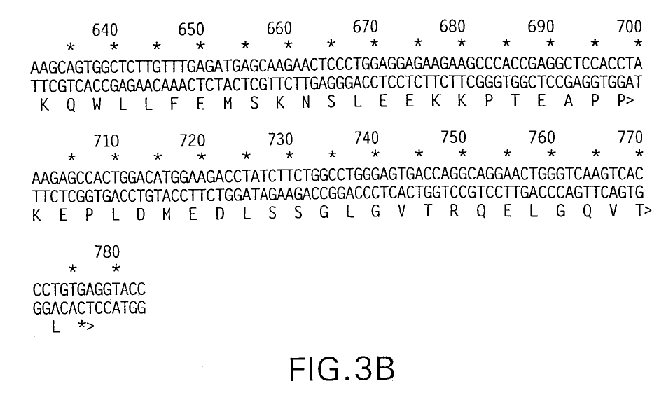

A DNA construct containing the cytoplasmic domain, transmembrane domain and a portion of the luminal domain of Ii fused to the MHC class II epitope string shown in FIG. 2 and amino acid residues 101 to 215 of Ii encoding the trimerization region of Ii was generated (FIG. 3). This construct, designated IiThfull, encodes the first 80 amino acids of invariant chain followed by the MHC class II epitope string (replacing CLIP) and the rest of the invariant chain (amino acids 101-215). Briefly, the construct was generated as two overlapping halves that were annealed and extended by PCR to yield the final product.

The 5′ end of IiThfull was made by amplifying 180T with murli-F (SEQ ID NO:40) and Th-Pad-R. The Th-Pad-R primer AGCGGCAGCCTTCAGGGTC (SEQ ID NO:51) corresponds to nucleotides 429-411. The 3′ half was made by amplifying bli#3 with IiPADRE-F and murli-R (SEQ ID NO:41). The IiPADRE-F primer GGCTGCCTGGACCCTGAAGGCTGCCGCTATGTCCATGGATAAC (SEQ ID NO:43) corresponds to nucleotides 402-444. Each PCR product was gel purified and mixed, then denatured, annealed, and extended by five cycles of PCR. Primers murli-F (SEQ ID NO:40) and murli-R (SEQ ID NO:41) were added and another 25 cycles performed. The full length product was gel purified, cloned, and sequenced.

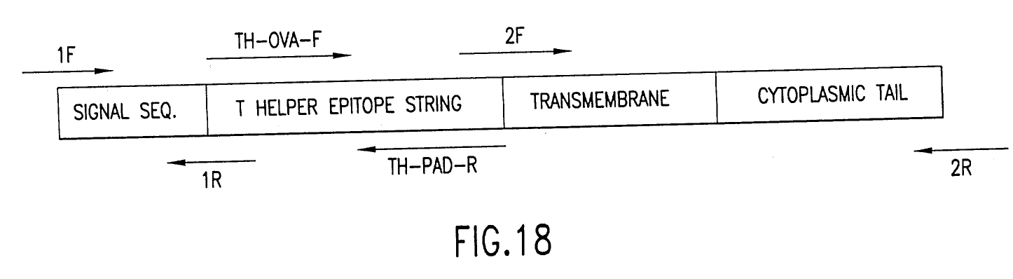

All of the remaining constructs described below were made essentially according to the scheme shown in FIG. 18. Briefly, primer pairs 1F plus 1R, designated below for each specific construct, were used to amplify the specific signal sequence and contained an overlapping 15 base pair tail identical to the 5′ end of the MHC class II epitope string. Primer pair Th-ova-F, ATCAGCCAGGCTGTGCACGC (SEQ ID NO:53), plus Th-Pad-R (SEQ ID NO:51) were used to amplify the MHC class II epitope string. A 15 base pair overlap and the specific transmembrane and cytoplasmic tail containing the targeting signals were amplified with primer pairs 2F plus 2R.

All three pieces of each cDNA were amplified using the following conditions: 1 cycle of 95° C. for 5 minutes, followed by 30 cycles of 95° C. for 15 seconds, 52° C. for 30 seconds, and 72° C. for 1 minute. Each of the three fragments was agrose-gel purified, and the signal sequence and MHC class II string fragments were combined and joined by five cycles in a second PCR. After five cycles, primers 1F and Th-Pad-R were added for 25 additional cycles and the PCR product was gel purified. This signal sequence plus MHC class II epitope string fragment was combined with the transmembrane plus cytoplasmic tail fragment for the final PCR. After five cycles, primers 1F plus 2R were added for 25 additional cycles and the product was gel purified, cloned and sequenced.

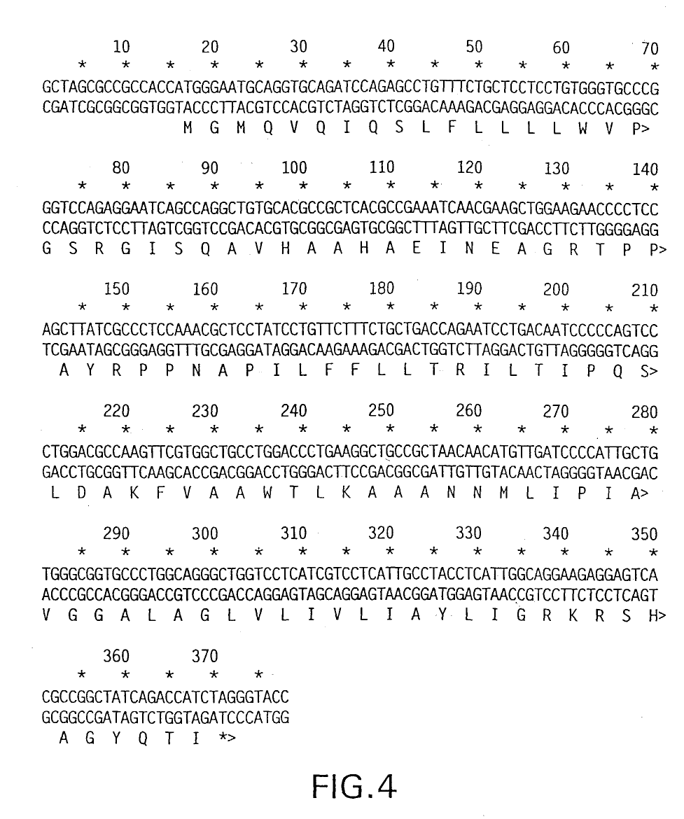

A DNA construct containing the murine immunoglobulin kappa signal sequence fused to the T helper epitope string shown in FIG. 2 and the transmembrane and cytoplasmic domains of LAMP-1 was generated (FIG. 4) (Granger et al., J. Biol. Chem. 265:12036-12043 (1990)), which is incorporated by reference (mouse LAMP-1 GenBank accession No. M32015). This construct, designated kappaLAMP-Th, contains the consensus mouse immunoglobulin kappa signal sequence and was amplified from a plasmid containing full length immunoglobulin kappa as depicted in FIG. 18. The primer 1F used was the oligonucleotide designated KappaSig-F, GCTAGCGCCGCCACCATGGGAATGCAG (SEQ ID NO:54). The primer 1R used was the oligonucleotide designated Kappa-Th-R, CACAGCCTGGCTGATTCCTCTGGACCC (SEQ ID NO:55). The primer 2F used was the oligonucleotide designated PAD/LAMP-F, CTGAAGGCTGCCGCTAACAACATGTTGATCCCC (SEQ ID NO:56). The primer 2R used was the oligonucleotide designated LAMP-CYTOR, GGTACCCTAGATGGTCTGATAGCC (SEQ ID NO:57).

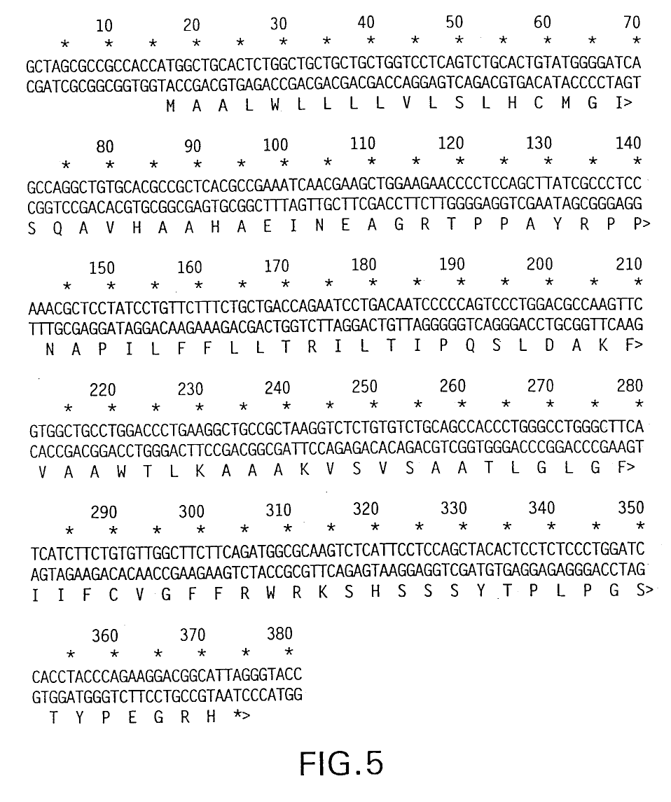

A DNA construct containing the signal sequence of H2-M fused to the MHC class II epitope string shown in FIG. 2 and the transmembrane and cytoplasmic domains of H2-M was generated (FIG. 5). The mouse H2-M gene has been described previously, Peleraux et al., Immunogenetics 43:204-214 (1996)), which is incorporated herein by reference. This construct was designated H2M-Th and was constructed as depicted in FIG. 18. The primer 1F used was the oligonucleotide designated H2-Mb-1F, GCC GCT AGC GCC GCC ACC ATG GCT GCA CTC TGG (SEQ ID NO:58). The primer 1R used was the oligonucleotide designated H2-Mb-1R, CAC AGC CTG GCT GAT CCC CAT ACA GTG CAG (SEQ ID NO:59). The primer 2F used was the oligonucleotide designated H2-Mb-2F, CTG AAG GCT GCC GCT AAG GTC TCT GTG TCT (SEQ ID NO:60). The primer 2R used was the oligonucleotide designated H2-Mb-2R, GCG GGT ACC CTA ATG CCG TCC TTC (SEQ ID NO:61).

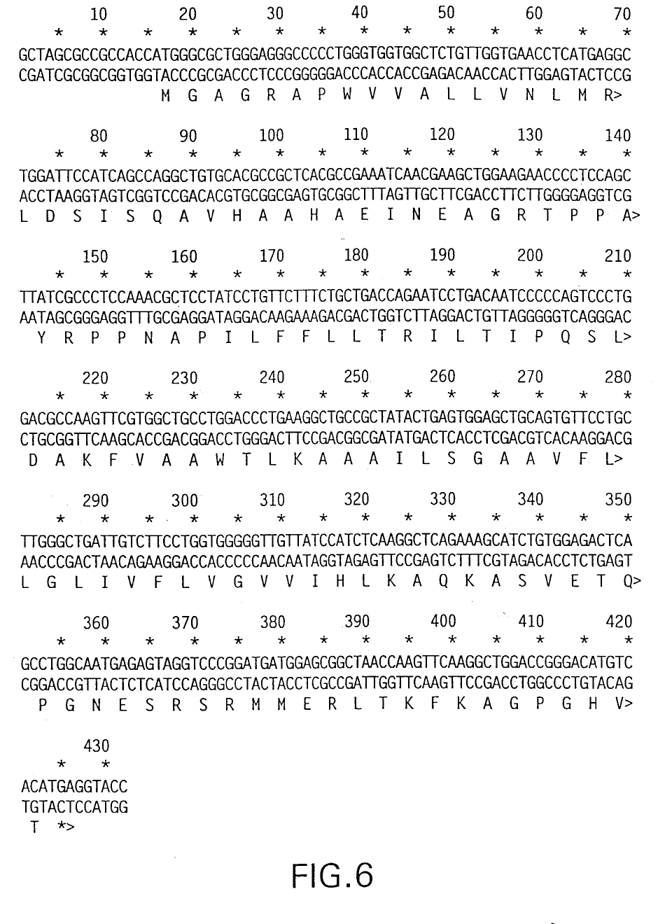

A DNA construct containing the signal sequence of H2-DO fused to the MHC class II epitope string shown in FIG. 2 and the transmembrane and cytoplasmic domains of H2-DO was generated (FIG. 6). The mouse H2-DO gene has been described previously (Larhammar et al., J. Biol. Chem. 260:14111-14119 (1985)), which is incorporated herein by reference (GenBank accession No. M19423). This construct, designated H₂O-Th, was constructed as depicted in FIG. 18. The primer 1F used was the oligonucleotide designated H2-Ob-1F, GCG GCT AGC GCC GCC ACC ATG GGC GCT GGG AGG (SEQ ID NO:62). The primer 1R used was the oligonucleotide designated H2-Ob-1R, TGC ACA GCC TGG CTG ATG GAA TCC AGC CTC (SEQ ID NO:63). The primer 2F used was the oligonucleotide designated H2-Ob-2F, CTG AAG GCT GCC GCT ATA CTG AGT GGA GCT (SEQ ID NO:64). The primer 2R used was the oligonucleotide designated H2-Ob-2R, GCC GGT ACC TCA TGT GAC ATG TCC CG (SEQ ID NO:65).

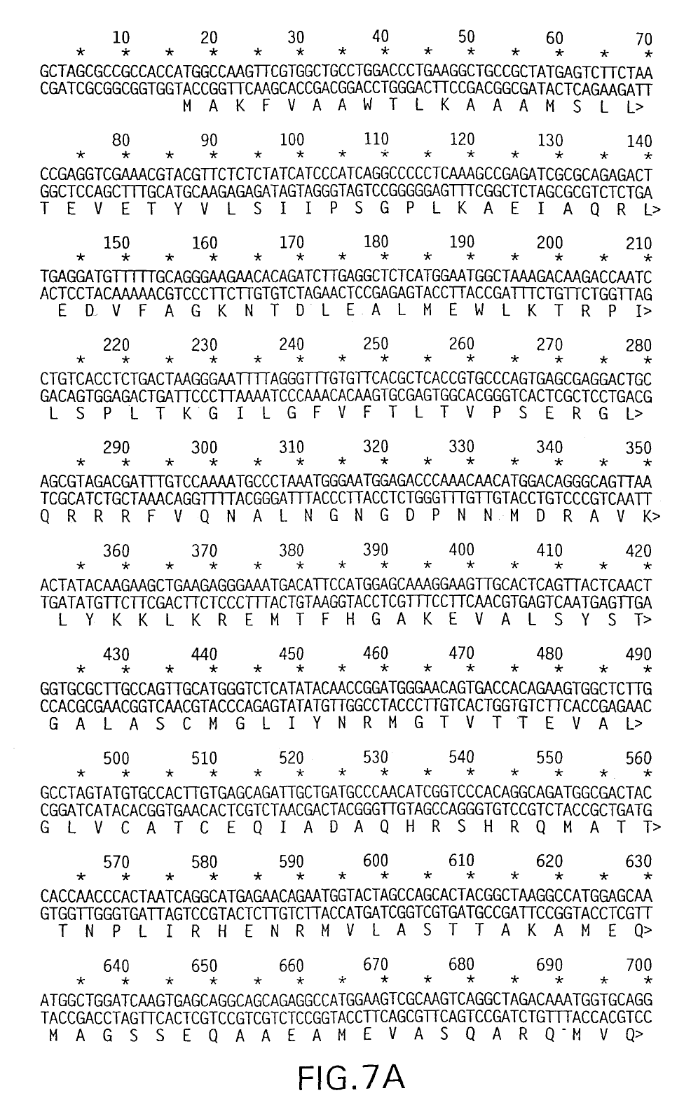

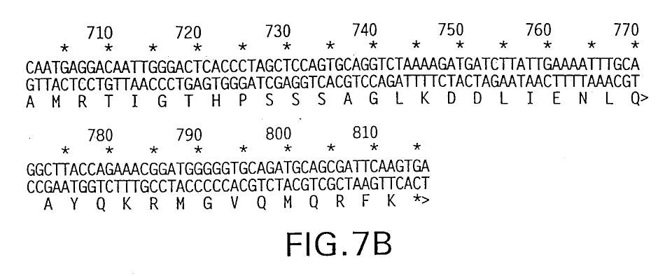

A DNA construct containing a pan DR epitope sequence (SEQ ID NO:52) fused to the amino-terminus of influenza matrix protein is generated (FIG. 7). This construct, designated PADRE-Influenza matrix, contains the universal MHC class II epitope PADRE attached to the amino terminus of the influenza matrix coding sequence. The construct is made using a long primer on the 5′ end primer. The 5′ primer is the oligonucleotide GCTAGCGCCGCCACCATGGCCAAGTTCGTGGCTGCCTGGACCCTGAAGGCTGCCGCT ATGAGTCTTCTAACCGAGGTCGA (SEQ ID NO:66). The 3′ primer is the oligonucleotide TCACTTGAATCGCTGCATCTGCACCCCCAT (SEQ ID NO:67). Influenza virus from the America Type Tissue Collection (ATCC) is used as a source for the matrix coding region (Perdue et al. Science 279:393-396 (1998)), which is incorporated herein by reference (GenBank accession No. AF036358).

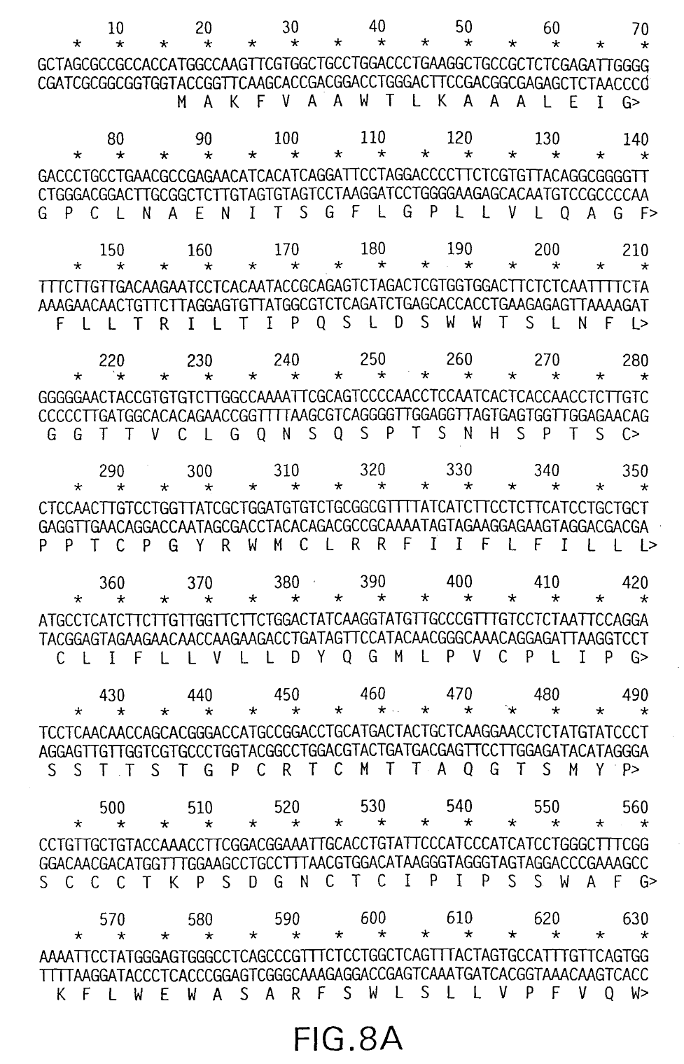

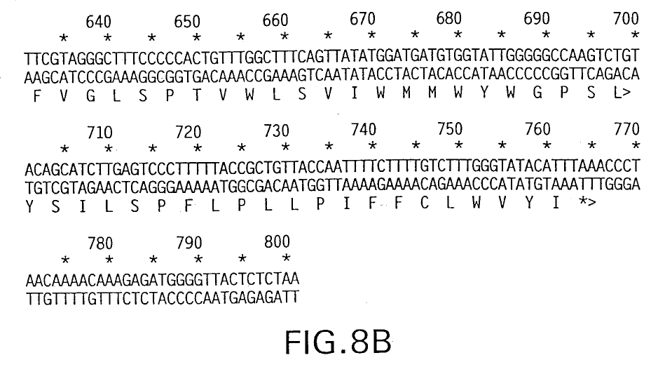

A DNA construct containing a pan DR epitope sequence (SEQ ID NO:52) fused to the amino-terminus of HBV-S antigen was generated (FIG. 8). This construct is designated PADRE-HBV-s and was generated by annealing two overlapping oligonucleotides to add PADRE onto the amino terminus of hepatitis B surface antigen (Michel et al., Proc. Natl. Acad. Sci. USA 81:7708-7712 (1984); Michel et al., Proc. Natl. Acad. Sci. USA 92:5307-5311 (1995)), each of which is incorporated herein by reference. One oligonucleotide was GCTAGCGCCGCCACCATGGCCAAGTTCGTGGCTGCCTGGACCCTGAAGGCTGCCGCT C (SEQ ID NO:68). The second oligonucleotide was CTCGAGAGCGGCAGCCTTCAGGGTCCAGGCAGCCACGAACTTGGCCATGGTGGCGG CG (SEQ ID NO:69). When annealed, the oligos have NheI and XhoI cohesive ends. The oligos were heated to 100° C. and slowly cooled to room temperature to anneal. A three part ligation joined PADRE with an XhoI-KpnI fragment containing HBV-s antigen into the NheI plus KpnI sites of the expression vector.

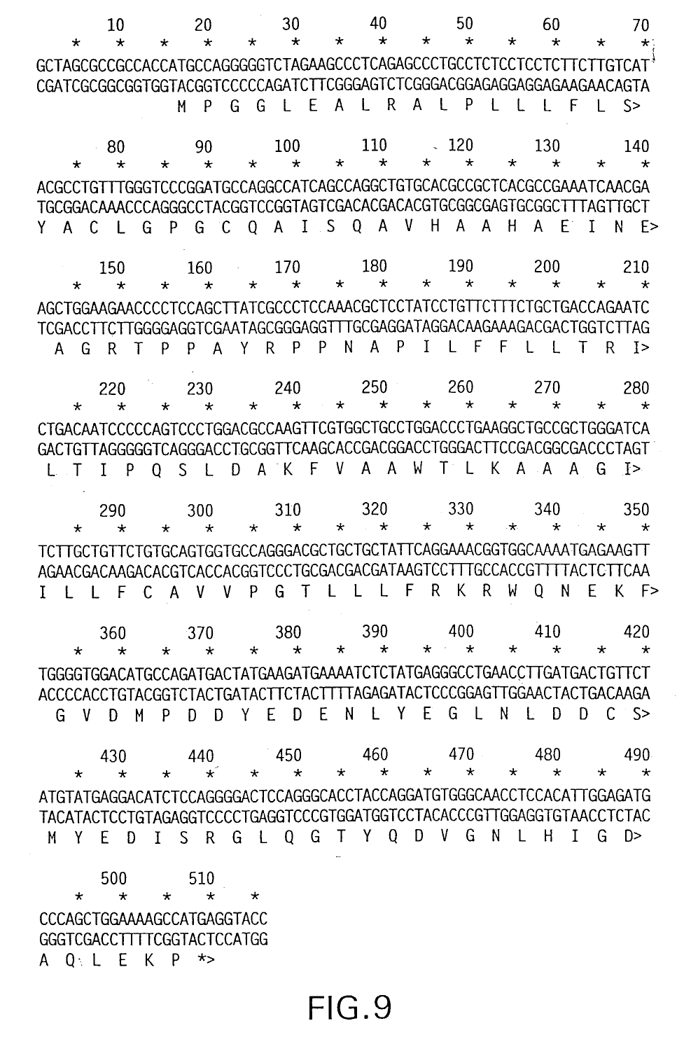

A DNA construct containing the signal sequence of Ig-α fused to the MHC class II epitope string shown in FIG. 2 and the transmembrane and cytoplasmic domains of Ig-α was generated (FIG. 9). The mouse Ig-α gene has been described previously (Kashiwamura et al., J. Immunol. 145:337-343 (1990)), which is incorporated herein by reference (GenBank accession No. M31773). This construct, designated Ig-alphaTh, was constructed as depicted in FIG. 18. The primer 1F used was the oligonucleotide designated Ig alpha-1 F, GCG GCT AGC GCC GCC ACC ATG CCA GGG GGT CTA (SEQ ID NO:70). The primer 1R used was the oligonucleotide designated Igalpha-1R, GCA CAG CCT GGC TGA TGG CCT GGC ATC CGG (SEQ ID NO:71). The primer 2F used was the oligonucleotide designated Igalpha-2F, CTG AAG GCT GCC GCT GGG ATC ATC TTG CTG (SEQ ID NO:72). The primer 2R used was the oligonucleotide designated Igalpha-2R, GCG GGT ACC TCA TGG CTT TTC CAG CTG (SEQ ID NO:73).

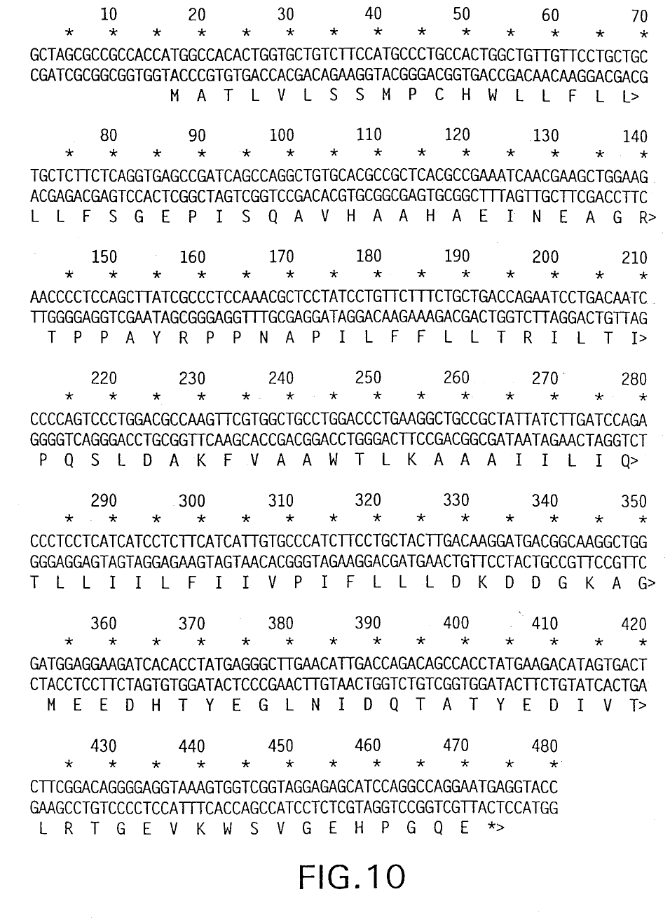

A DNA construct containing the signal sequence of Ig-β fused to the MHC class II string shown in FIG. 2 and the transmembrane and cytoplasmic domains of Igβ was generated (FIG. 10). The Ig-β sequence is the B29 gene of mouse and has been described previously (Hermanson et al., Proc. Natl. Acad. Sci. USA 85:6890-6894 (1988)), which is incorporated herein by reference (GenBank accession No. J03857). This construct, designated Ig-betaTh, was constructed as depicted in FIG. 18. The primer 1F used was the oligonucleotide designated B29-1F (33mer) GCG GCT AGC GCC GCC ACC ATG GCC ACA CTG GTG (SEQ ID NO:74). The primer 1R used was the oligonucleotide designated B29-1R (30mer) CAC AGC CTG GCT GAT CGG CTC ACC TGA GAA (SEQ ID NO:75). The primer 2F used was the oligonucleotide designated B292F (30mer) CTG AAG GCT GCC GCT ATT ATC TTG ATC CAG (SEQ ID NO:76). The primer 2R used was the oligonucleotide designated B29-2R (27mer), GCC GGT ACC TCA TTC CTG GCC TGG ATG (SEQ ID NO:77).

A DNA construct containing the signal sequence of the kappa immunoglobulin signal sequence fused to the MHC class II epitope string shown in FIG. 2 was constructed (FIG. 11). This construct is designated SigTh and was generated by using the kappaLAMP-Th construct (shown in FIG. 4) and amplifying with the primer pair KappaSig-F (SEQ ID NO:54) plus Help-epR (SEQ ID NO:47) to create SigTh. SigTh contains the kappa immunoglobulin signal sequence fused to the T helper epitope string and terminated with a translational stop codon.

Constructs encoding human sequences corresponding to the above described constructs having mouse sequences are prepared by substituting human sequences for the mouse sequences. Briefly, for the IiPADRE construct, corresponding to FIG. 1, amino acid residues 1-80 from the human Ii gene HLA-DR sequence (FIG. 12) (GenBank accession No. X00497 M14765) is substituted for the mouse Ii sequences, which is fused to PADRE, followed by human invanant chain HLA-DR amino acid residues 114-223. For the 180T construct, corresponding to FIG. 2, amino acid residues 1-80 from the human sequence of Ii is followed by a MHC class II epitope string. For the IiThfull construct, corresponding to FIG. 3, amino acid residues 1-80 from the human sequence of Ii, which is fused to a MHC class II epitope string, is followed by human invariant chain amino acid residues 114-223.

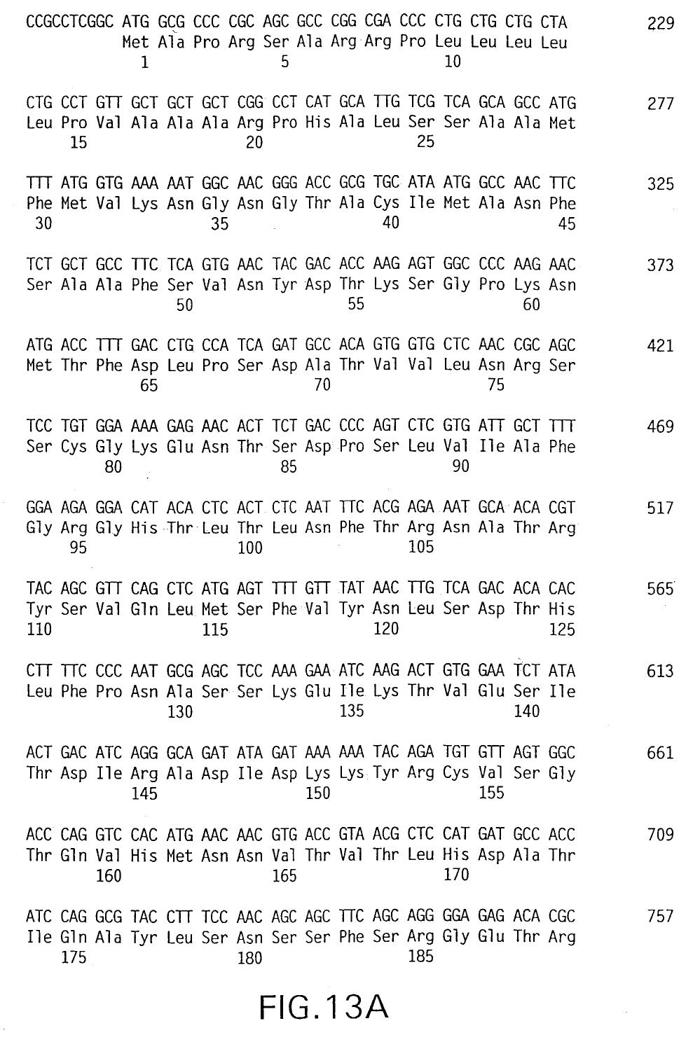

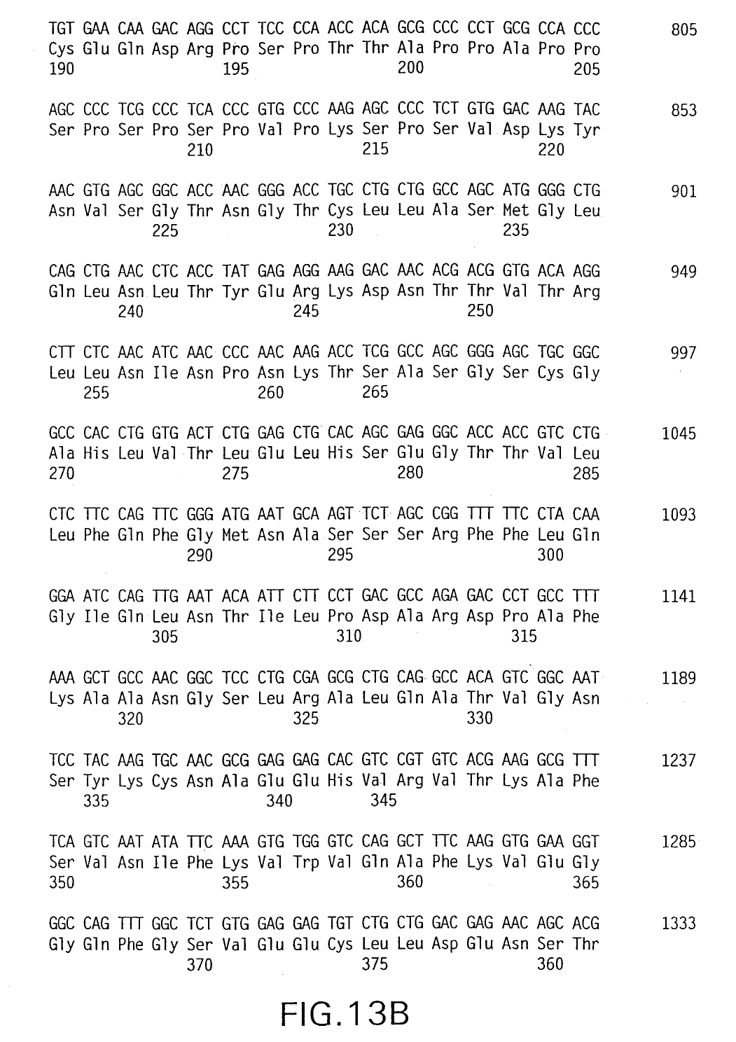

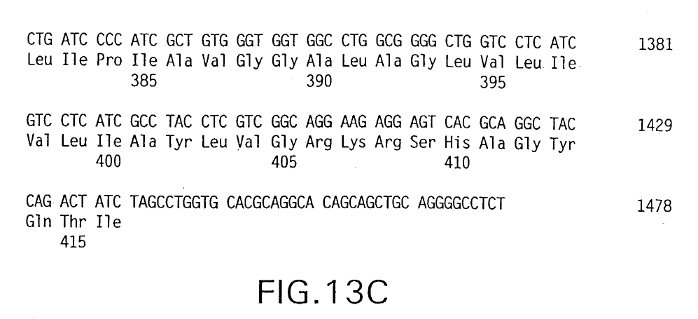

For the LAMP-Th construct, similar to FIG. 4, the signal sequence encoded by amino acid residues 1-19 (nucleotides 11-67) of human LAMP-1 (FIG. 13) (GenBank accession No. J04182), which is fused to the MHC class II epitope string, is followed by the transmembrane (nucleotides 1163-1213) and cytoplasmic tail (nucleotides 1214-1258) region encoded by amino acid residues 380-416 of human LAMP-1.

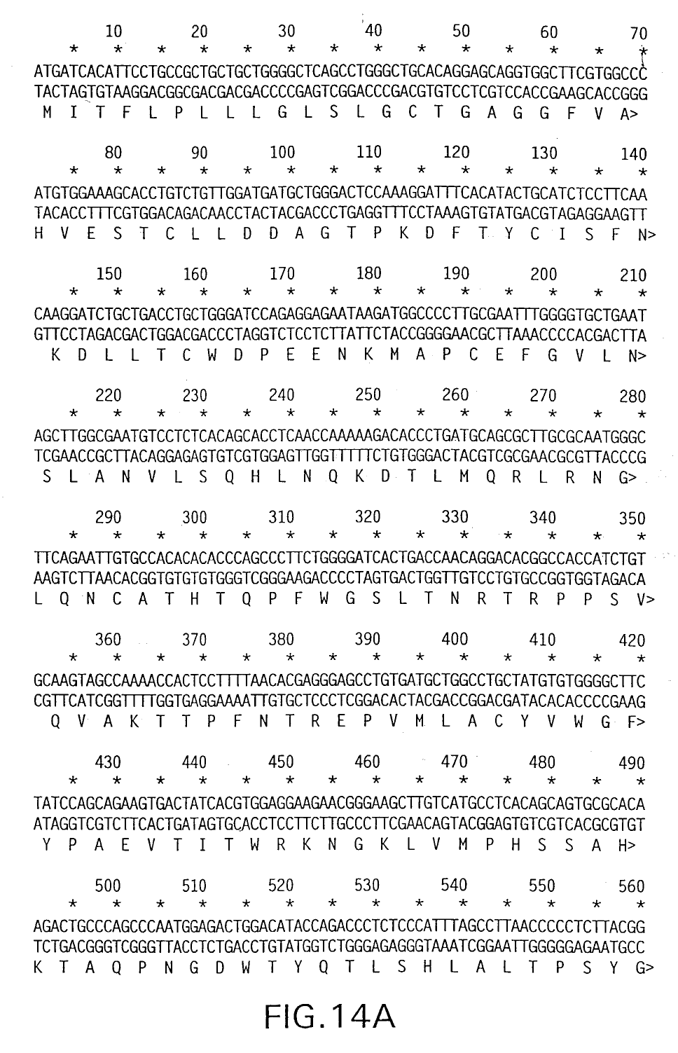

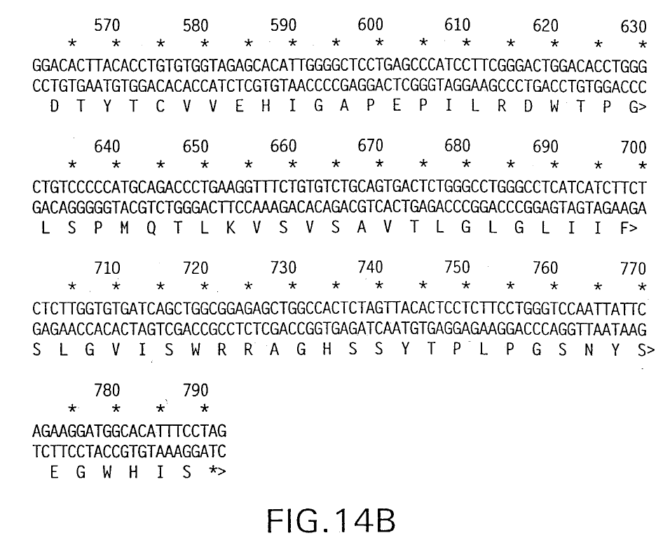

For the HLA-DM-Th construct, corresponding to FIG. 5, the signal sequence encoded by amino acid residues 1-17 (nucleotides 1-51) of human HLA-DMB (FIG. 14) (GenBank accession No. U15085), which is fused to the MHC class II epitope string, is followed by the transmembrane (nucleotides 646-720) and cytoplasmic tail (nucleotides 721-792) region encoded by amino acid residues 216-263 of human HLA-DMB.

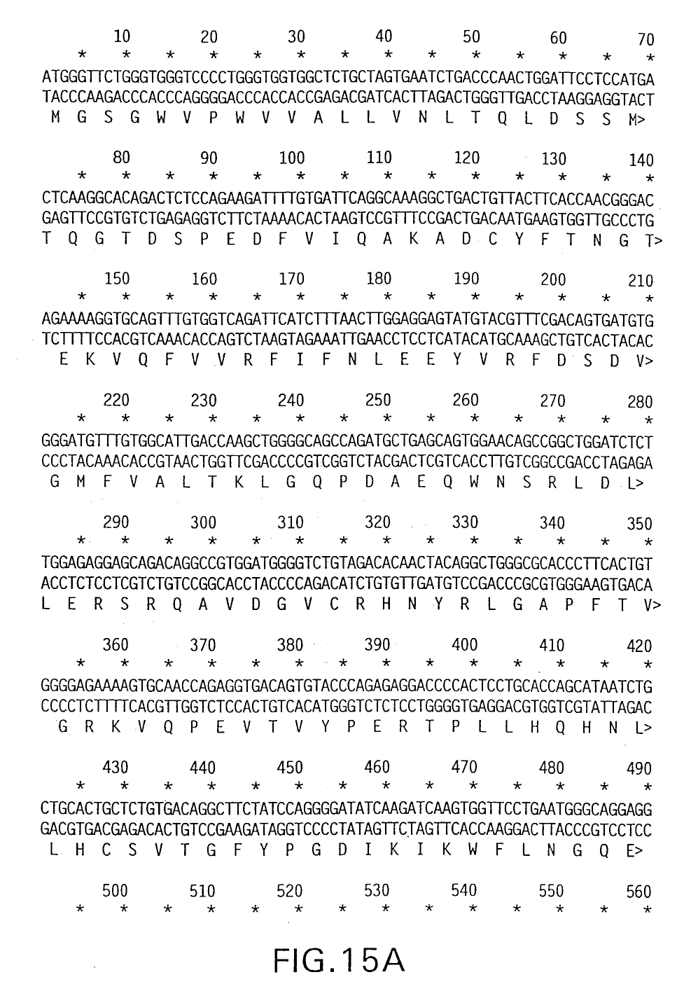

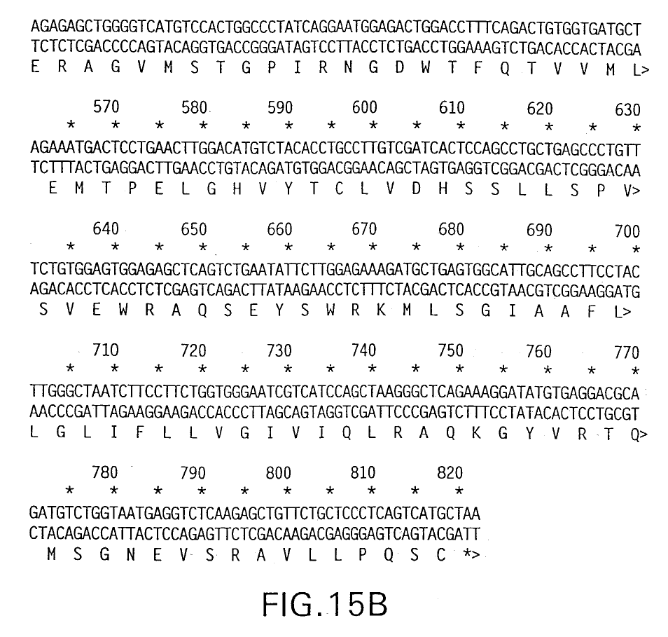

For the HLA-DO-Th construct, corresponding to FIG. 6, the signal sequence encoded by amino acid residues 1-21 (nucleotides 1-63) of human HLA-DO (FIG. 15) (GenBank accession No. L29472 J02736 N00052), which is fused to the MHC class II epitope string, is followed by the transmembrane (nucleotides 685-735) and cytoplasmic tail (nucleotides 736-819) region encoded by amino acid residues 223-273 of human HLA-DO.

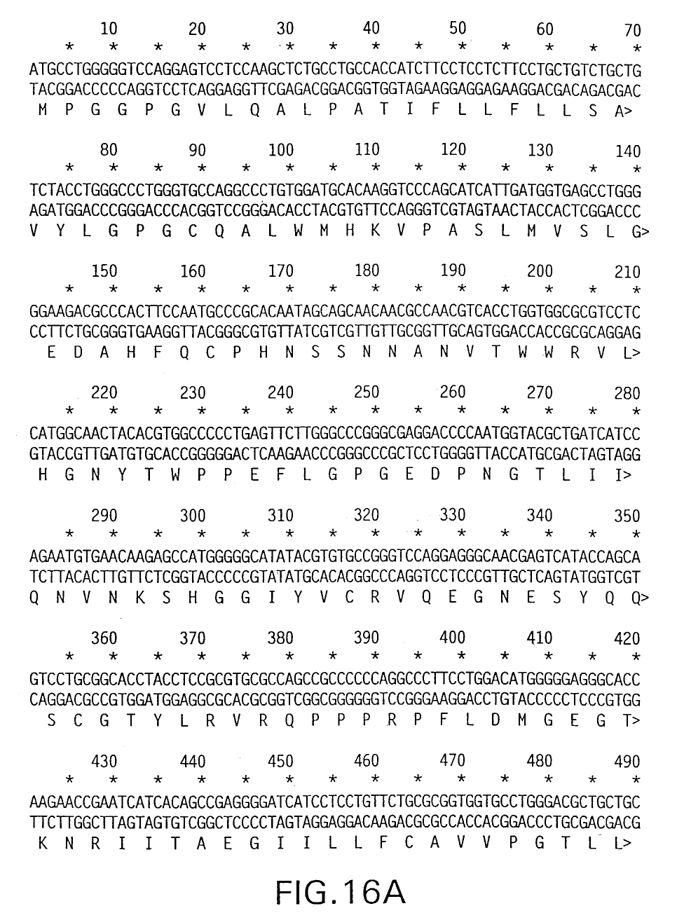

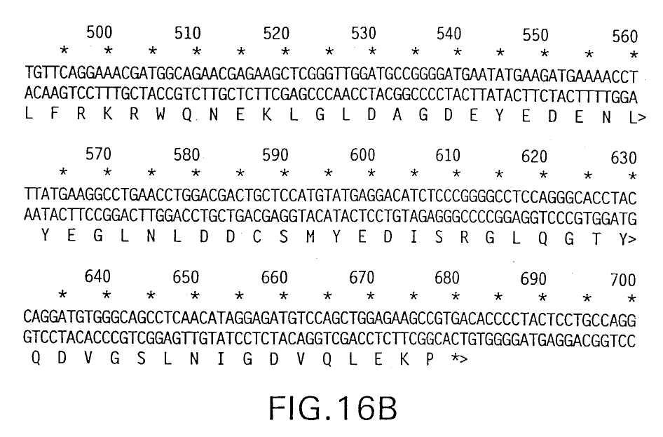

For the Ig-alphaTh construct, corresponding to FIG. 9, the signal sequence encoded by amino acid residues 1-29 (nucleotides 1-87) of human Ig-α MB-1 (FIG. 16) (GenBank accession No. U05259), which is fused to the MHC class II epitope string, is followed by the transmembrane (nucleotides 424-498) and cytoplasmic tail (nucleotides 499-678) region encoded by amino acid residues 142-226 of human Ig-α MB-1.

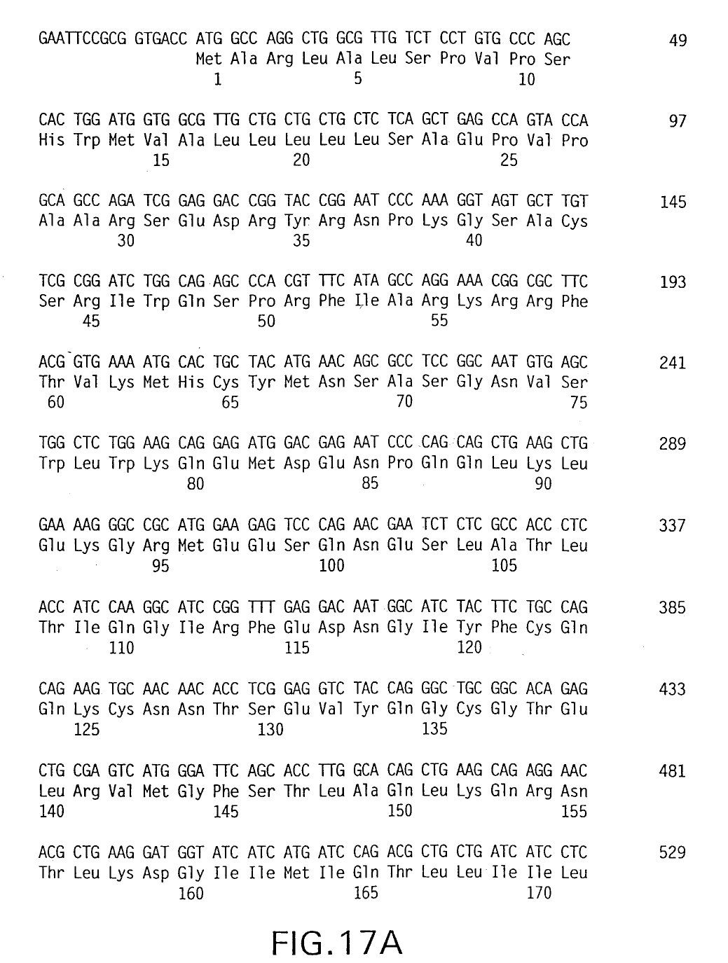

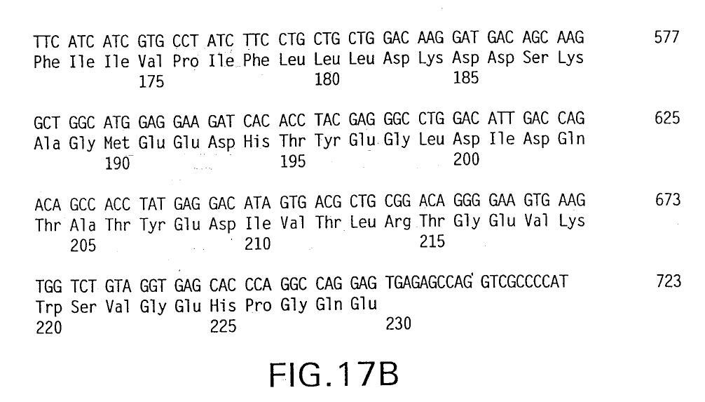

For the Ig-betaTh construct, corresponding to FIG. 10, the signal sequence encoded by amino acid residues 1-28 (nucleotides 17-100) of human Ig-β B29 (FIG. 17) (GenBank accession No. M80461), which is fused to the MHC class II epitope string, is followed by the transmembrane (nucleotides 500-547) and cytoplasmic tail (nucleotides 548-703) region encoded by amino acid residues 156-229 of human Ig-β.

The SigTh construct shown in FIG. 11 can be used in mouse and human. Alternatively, a signal sequence derived from an appropriate human gene containing a signal sequence can be substituted for the mouse kappa immunoglobulin sequence in the Sig Th construct.

The PADRE-Influenza matrix construct shown in FIG. 7 and the PADRE-HBVs construct shown in FIG. 8 can be used in mouse and human.

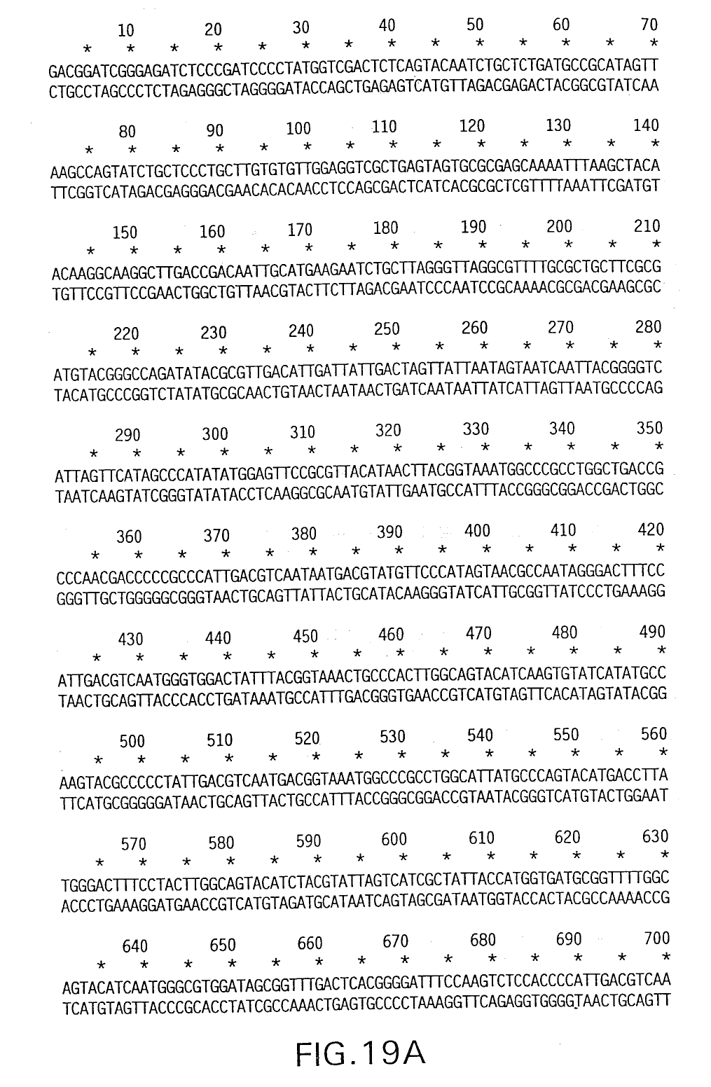

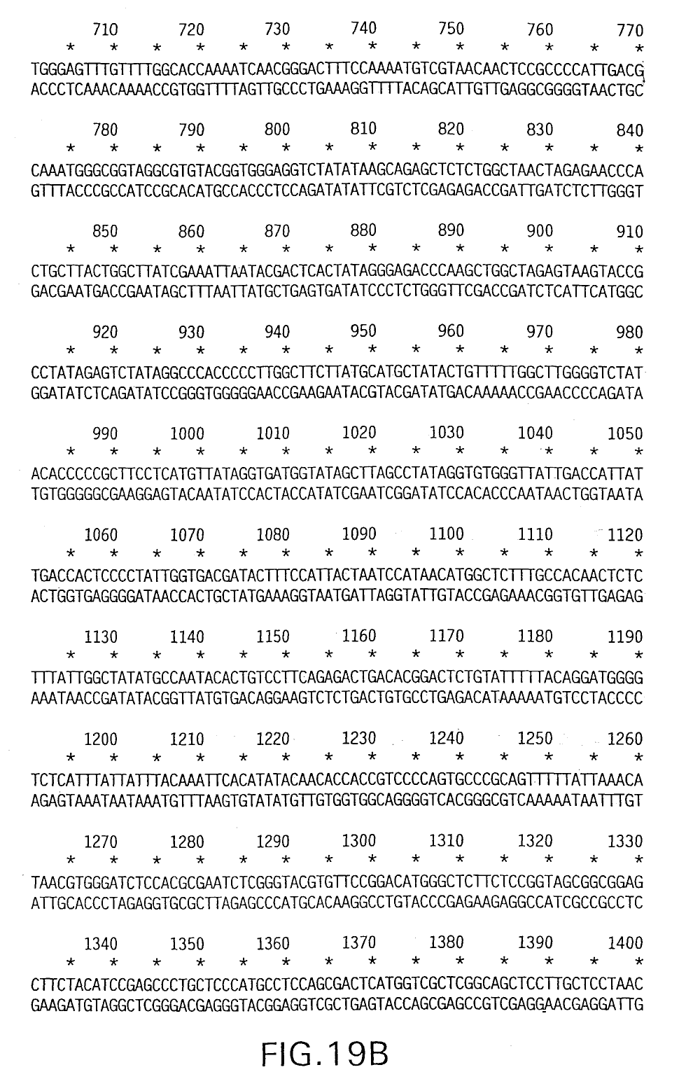

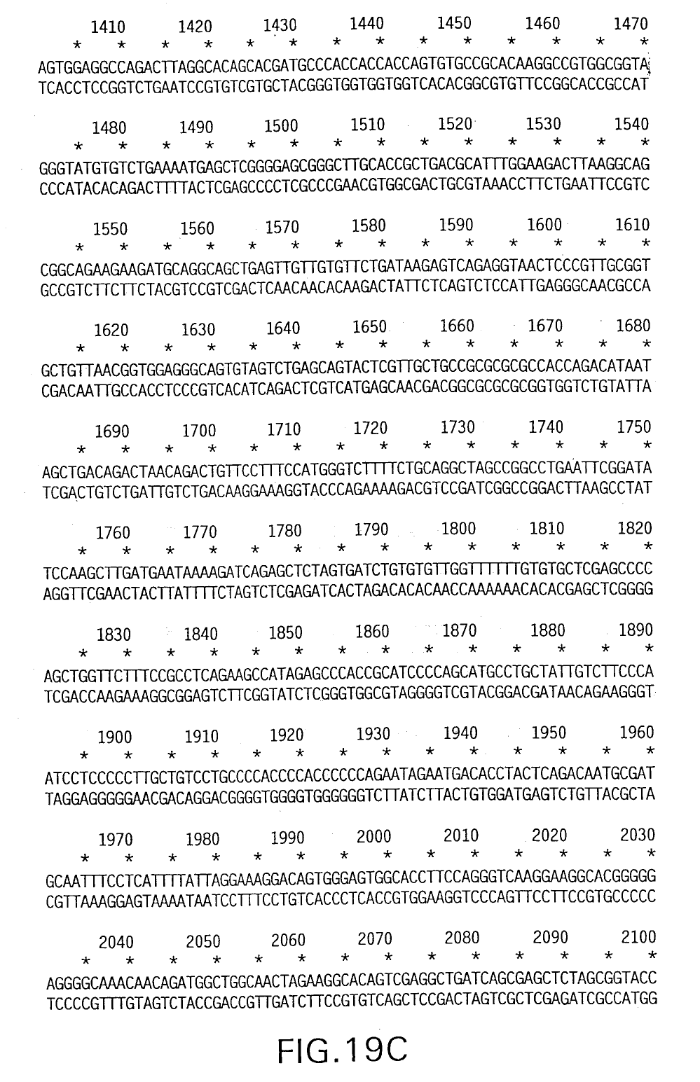

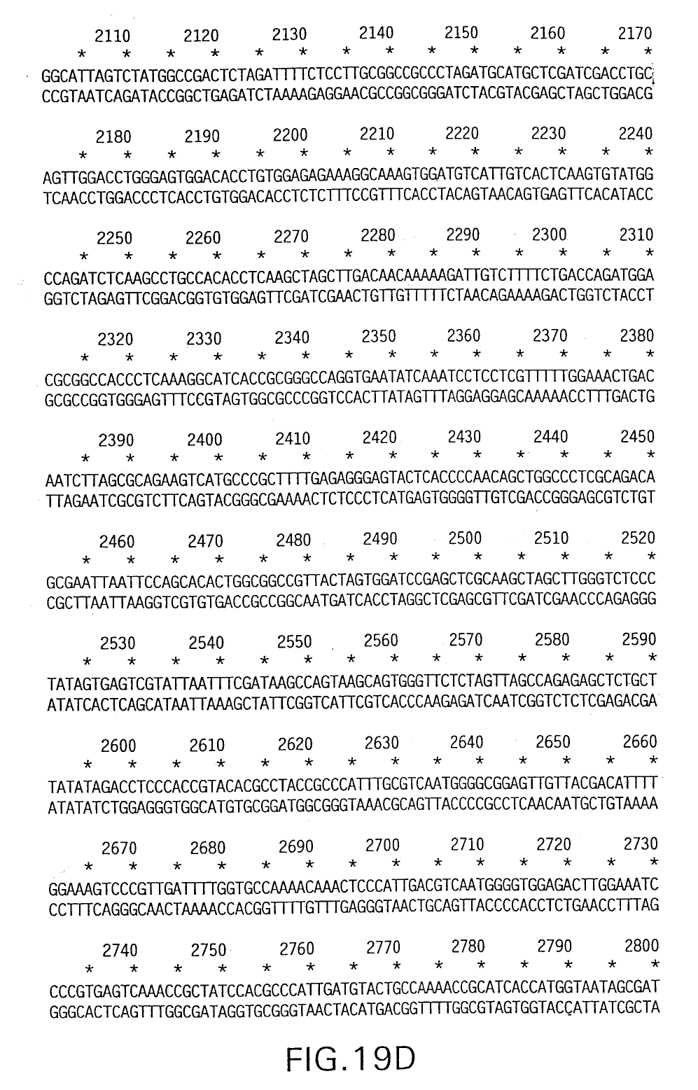

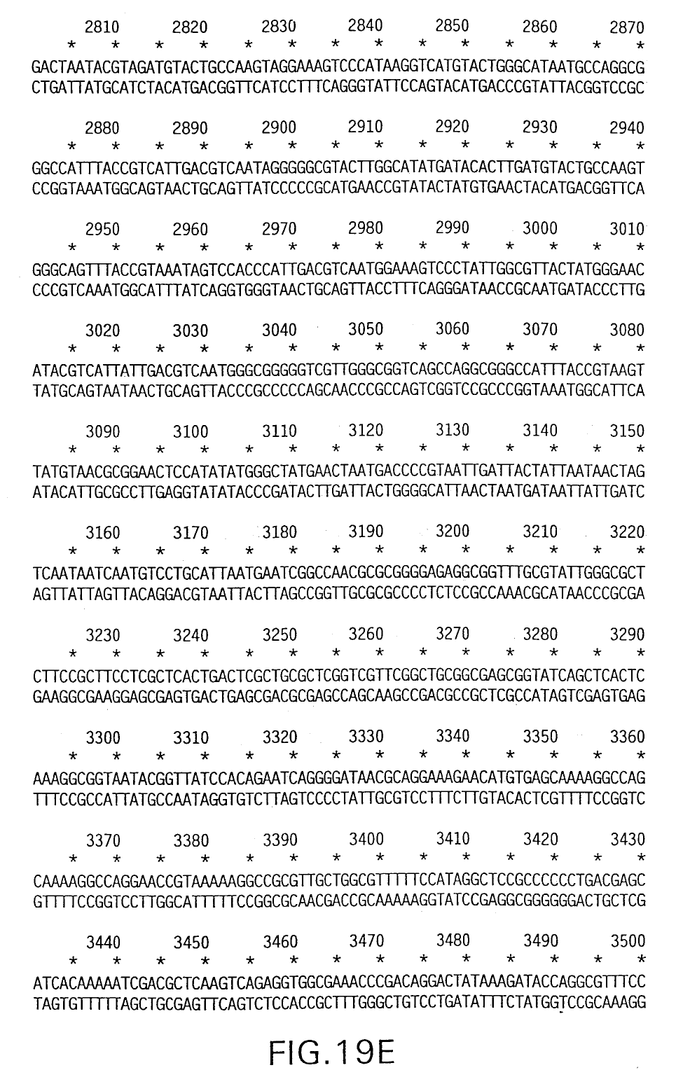

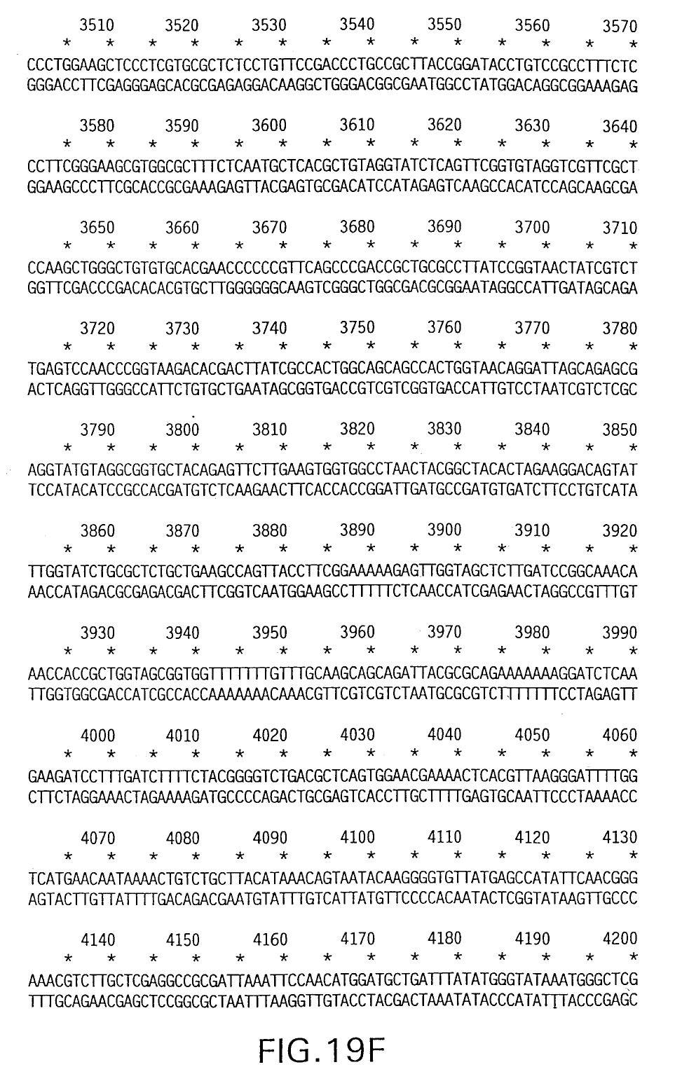

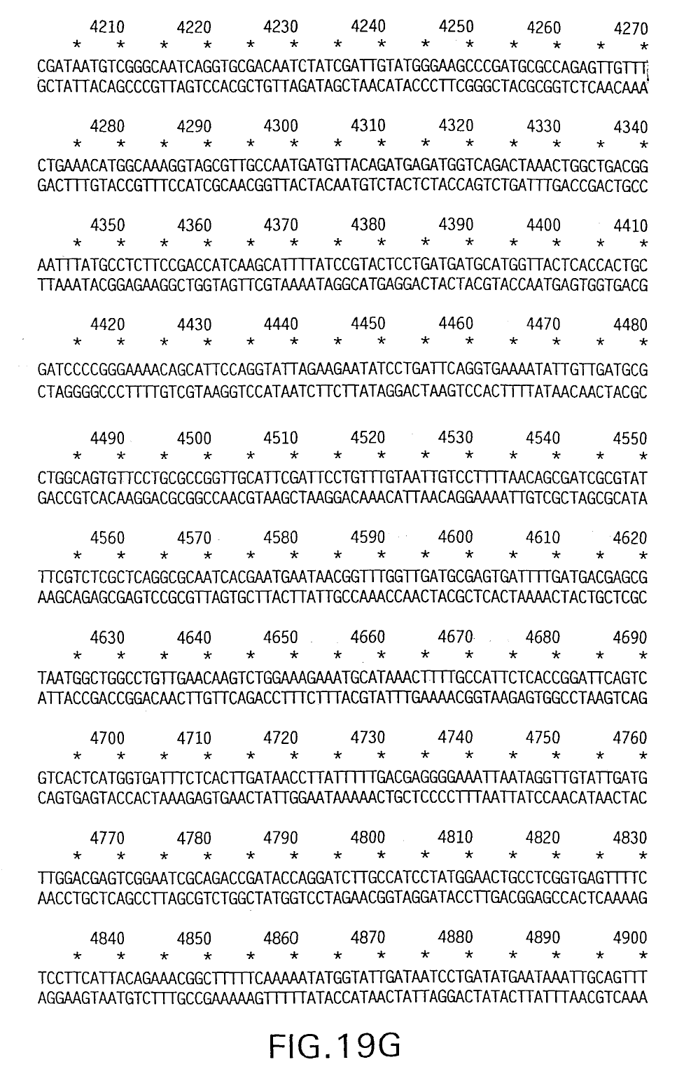

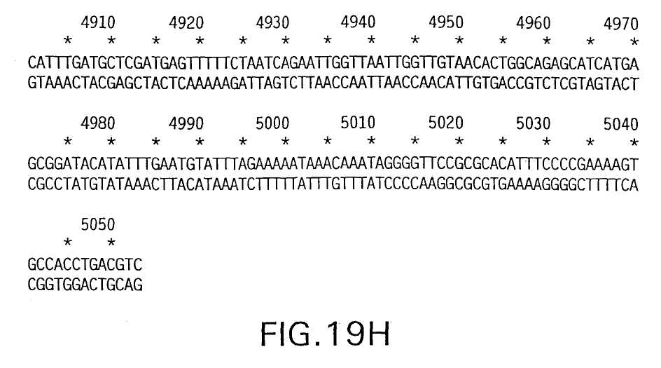

Some of the DNA constructs described above were cloned into the vector pEP2 (FIG. 19; SEQ ID NO:35). The pEP2 vector was constructed to contain dual CMV promoters. The pEP2 vector used the backbone of pcDNA3.1(−)Myc-His A from Invitrogen and pIRES1hyg from Clontech. Changes were made to both vectors before the CMV transcription unit from pIRES1hyg was moved into the modified pcDNA vector.

The pcDNA3.1(−)Myc-His A vector (http://www.invitrogen.com) was modified. Briefly, the PvuII fragment (nucleotides 1342-3508) was deleted. A BspHI fragment that contains the Ampicillin resistance gene (nucleotides 4404-5412) was cut out. The Ampicillin resistance gene was replaced with the kanamycin resistance gene from pUC4K (GenBank Accession #X06404). pUC4K was amplified with the primer set: TCTGATGTTACATTGCACAAG (SEQ ID NO:78) (nucleotides 1621-1601) and GCGCACTCATGATGCTCTGCCAGTGTTACAACC (SEQ ID NO:79) (nucleotides 682-702 plus the addition of a BspHI restriction site on the 5′ end). The PCR product was digested with BspHI and ligated into the vector digested with BspHI. The region between the PmeI site at nucleotide 905 and the EcoRV site at nucleotide 947 was deleted. The vector was then digested with PmeI (cuts at nucleotide 1076) and ApaI (cuts at nucleotide 1004), Klenow filled in at the cohesive ends and ligated. The KpnI site at nucleotide 994 was deleted by digesting with KpnI and filling in the ends with Klenow DNA polymerase, and ligating. The intron A sequence from CMV (GenBank accession M21295, nucleotides 635-1461) was added by amplifying CMV DNA with the primer set: GCGTCTAGAGTAAGTACCGCCTATAGACTC (SEQ ID NO:80) (nucleotides 635-655 plus an XbaI site on the 5′ end) and CCGGCTAGCCTGCAGAAAAGACCCATGGAA (SEQ ID NO:81) (nucleotides 1461-1441 plus an NheI site on the 3′ end). The PCR product was digested with XbaI and NheI and ligated into the NheI site of the vector (nucleotide 895 of the original pcDNA vector) so that the NheI site was on the 3′ end of the intron.

To modify the pIRES 1 hyg vector (GenBank Accession U89672, Clontech), the KpnI site (nucleotide 911) was deleted by cutting and filling in with Klenow. The plasmid was cut with NotI (nucleotide 1254) and XbaI (nucleotide 3196) and a polylinker oligo was inserted into the site. The polylinker was formed by annealing the following two oligos: GGCCGCAAGGAAAAAATCTAGAGTCGGCCATAGACTAATGCCGGTACCG (SEQ ID NO:82) and CTAGCGGTACCGGCATTAGTCTATGGCCCGACTCTAGATTTTTTCCTTGC (SEQ ID NO:83). The resulting plasmid was cut with HincII and the fragment between HincII sites 234 and 3538 was isolated and ligated into the modified pcDNA vector. This fragment contains a CMV promoter, intron, polylinker, and polyadenylation signal.

The pIREShyg piece and the pcDNA piece were combined to form pEP2. The modified pcDNA3.1(−)Myc-His A vector was partially digested with PvuII to isolate a linear fragment with the cut downstream of the pcDNA polyadenylation signal (the other PvuII site is the CMV intron). The HincII fragment from the modified pIRES1hyg vector was ligated into the PvuII cut vector. The polyadenylation signal from the pcDNA derived transcription unit was deleted by digesting with EcoRI (pcDNA nucleotide 955) and XhoI (pIRES1hyg nucleotide 3472) and replaced with a synthetic polyadenylation sequence. The synthetic polyadenylation signal was described in Levitt et al., Genes and Development 3:1019-1025 (1989)).

Two oligos were annealed to produce a fragment that contained a polylinker and polyadenylation signal with EcoRI and XhoI cohesive ends. The oligos were: AATTCGGATATCCAAGCTTGATGAATAAAAGATCAGAGCTCTAGTGATCTGTGTGTTG TGTGTGC (SEQ ID NO:84) and TCGAGCACACAAAAAACCAACACACAGATCACTAGAGCTCTGATCTTTTTATTCATC GATATCCG (SEQ ID NO:85).

The resulting vector is named pEP2 and contains two separate transcription units. Both transcription units use the same CMV promoter but each contains different intron, polylinker, and polyadenylation sequences.

The pEP2 vector contains two transcription units. The first transcription unit contains the CMV promoter initially from pcDNA (nucleotides 210-862 in FIG. 19), CMV intron A sequence (nucleotides 900-1728 in FIG. 19), polylinker cloning site (nucleotides 1740-1760 in FIG. 19) and synthetic polyadenylation signal (nucleotides 1764-1769 in FIG. 19). The second transcription unit, which was initially derived from pIRES1hyg, contains the CMV promoter (nucleotides 3165-2493 in FIG. 19), intron sequence (nucleotides 2464-2173 in FIG. 19), polylinker clone site (nucleotides 2126-2095 in FIG. 19) and bovine growth hormone polyadenylation signal (nucleotides 1979-1974 in FIG. 19). The kanamycin resistance gene is encoded in nucleotides 4965-4061 (FIG. 19).

The DNA constructs described above were digested with NheI and KpnI and cloned into the XbaI and KpnI sites of pEP2 (the second transcription unit).

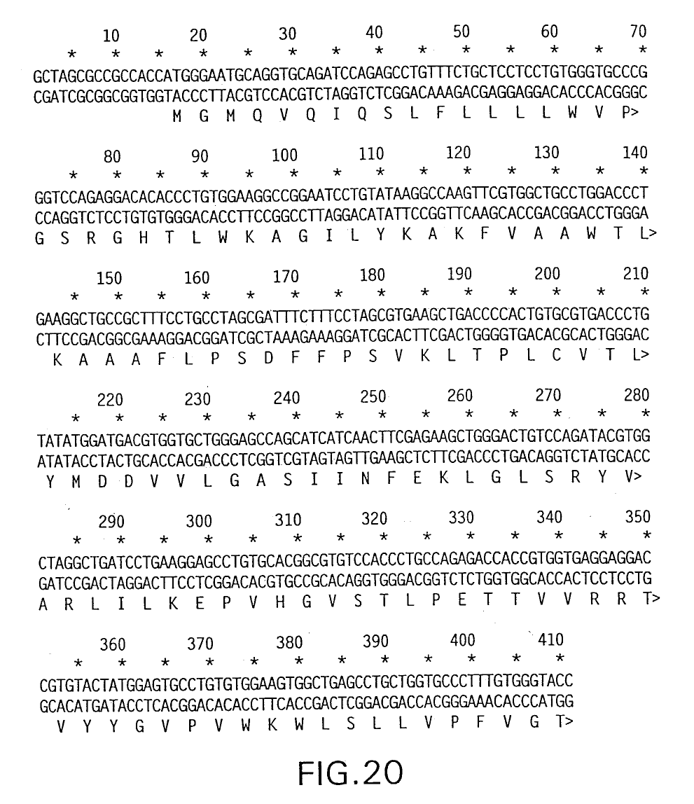

Additional vectors were also constructed. To test for the effect of co-expression of MHC class I epitopes with MHC class II epitopes, an insert was generated, designated AOS, that contains nine MHC class I epitopes. The AOS insert was initially constructed in the vector pMIN.0 (FIG. 20; SEQ ID NO:36). Briefly, the AOS insert contains nine MHC class I epitopes, six restricted by HLA-A2 and three restricted by HLA-Al 1, and the universal MHC class II epitope PADRE. The vector pMIN.0 contains epitopes from HBV, HIV and a mouse ovalbumin epitope. The MHC class I epitopes appear in pMIN.0 in the following order: consensus mouse Ig Kappa signal sequence (pMIN.0 amino acid residues 1-20, nucleotides 16--81) MQVQIQSLFLLLLWVPGSRG (SEQ ID NO:86) encoded by nucleotides ATG CAG GTG CAG ATC CAG AGC CTG TTT CTG CTC CTC CTG TGG GTG CCC GGG TCC AGA GGA (SEQ ID NO:87); HBV pol 149-159 (A11 restricted) (pMIN.0 amino acid residues 21-31, nucleotides 82-114) HTLWKAGILYK (SEQ ID NO:88) encoded by nucleotides CAC ACC CTG TGG AAG GCC GGA ATC CTG TAT AAG (SEQ ID NO:89); PADRE-universal MHC class II epitope (pMIN.0 amino acid residues 32-45, nucleotides 115-153) AKFVAAWTLKAAA (SEQ ID NO:52) encoded by nucleotides GCC AAG TTC GTG GCT GCC TGG ACC CTG AAG GCT GCC GCT (SEQ ID NO:90); HBV core 18-27 (A2 restricted) (pMIN.0 amino acid residues 46-55, nucleotides 154-183) FLPSDFFPSV (SEQ ID NO:91) encoded by nucleotides TTC CTG CCT AGC GAT TTC TTT CCT AGC GTG (SEQ ID NO:92); HIV env 120-128 (A2 restricted) (pMIN.0 amino acid residues 56-64, nucleotides 184-210) KLTPLCVTL (SEQ ID NO:93) encoded by nucleotides AAG CTG ACC CCA CTG TGC GTG ACC CTG (SEQ ID NO:94); HBV pol 551-559 (A2 restricted) (pMIN.0 amino acid residues 65-73, nucleotides 211-237) YMDDVVLGA (SEQ ID NO:95) encoded by nucleotides TAT ATG GAT GAC GTG GTG CTG GGA GCC (SEQ ID NO:96); mouse ovalbumin 257-264 (K restricted) (pMIN.0 amino acid residues 74-81, nucleotides 238-261) SIINFEKL (SEQ ID NO:97) encoded by nucleotides AGC ATC ATC AAC TTC GAG AAG CTG (SEQ ID NO:98); HBV pol 455-463 (A2 restricted) (pMIN.0 amino acid residues 82-90, nucleotides 262-288) GLSRYVARL (SEQ ID NO:99) encoded by nucleotides GGA CTG TCC AGA TAC GTG GCT AGG CTG (SEQ ID NO:100); HIV pol 476-84 (A2 restricted) (pMIN.0 amino acid residues 91-99, nucleotides 289-315) ILKEPVHGV (SEQ ID NO:101) encoded by nucleotides ATC CTG AAG GAG CCT GTG CAC GGC GTG (SEQ ID NO:102); HBV core 141-151 (All restricted) (pMIN.0 amino acid residues 100-110, nucleotides 316-348) STLPETTVVRR (SEQ ID NO:103) encoded by nucleotides TCC ACC CTG CCA GAG ACC ACC GTG GTG AGG AGA (SEQ ID NO:104); HIV env 49-58 (A11 restricted) (pMIN.0 amino acid residues 111-120, nucleotides 349-378) TVYYGVPVWK (SEQ ID NO:105) encoded by nucleotides ACC GTG TAC TAT GGA GTG CCT GTG TGG AAG (SEQ ID NO:106); and HBV env 335-343 (A2 restricted) (pMIN.0 amino acid residues 121-129, nucleotides 378-405) WLSLLVPFV (SEQ ID NO:107) encoded by nucleotides TGG CTG AGC CTG CTG GTG CCC TTT GTG (SEQ ID NO:108).

The pMIN.0 vector contains a KpnI restriction site (pMIN.0 nucleotides 406-411) and a NheI restriction site (pMIN.0 nucleotides 1-6). The pMIN.0 vector contains a consensus Kozak sequence (nucleotides 7-18) (GCCGCCACCATG; SEQ ID NO:109) and murine Kappa Ig-light chain signal sequence followed by a string of 10 MHC class I epitopes and one universal MHC class II epitope. The pMIN.0 sequence encodes an open reading frame fused to the Myc and His antibody epitope tag coded for by the pcDNA 3.1 Myc-His vector. The pMIN.0 vector was constructed with eight oligonucleotides: Min1 oligo GAGGAGCAGAAACAGGCTCTGGATCTGCACCTGCATTCCCATGGTGGCGGCGCTAGC AAGCTTCTTGCGC (SEQ ID NO:110); Min2 oligo CCTGTTTCTGCTCCTCCTGTGGGTGCCCGGGTCCAGAGGACACACCCTGTGGAAGGC CGGAATCCTGTATA (SEQ ID NO:111); Min3 oligo TCGCTAGGCAGGAAAGCGGCAGCCTTCAGGGTCCAGGCAGCCACGAACTTGGCCTT ATACAGGATTCCGG (SEQ ID NO:112); Min4 oligo CTTTCCTGCCTAGCGATTTCTTTCCTAGCGTGAAGCTGACCCCACTGTGCGTGACCCT GTATATGGATGAC (SEQ ID NO:113); Min5 oligo CGTACCTGGACAGTCCCAGCTTCTCGAAGTTGATGATGCTGGCTCCCAGCACCACGT CATCCATATACAG (SEQ ID NO:114); Min6 oligo GGACTGTCCAGATACGTGGCTAGGCTGATCCTGAAGGAGCCTGTGCACGGCGTGTCC ACCCTGCCAGAGAC (SEQ ID NO:115); Min7 oligo GCTCAGCCACTTCCACACAGGCACTCCATAGTACACGGTCCTCCTCACCACGGTGGT CTCTGGCAGGGTG (SEQ ID NO:116); Min8 oligo GTGGAAGTGGCTGAGCCTGCTGGTGCCCTTTGTGGGTACCTGATCTAGAGC (SEQ ID NO:117).

Additional primers were flanking primer 5′, GCG CAA GAA GCT TGC TAG CG (SEQ ID NO:118) and flanking primer 3′, GCT CTA GAT CAG GTA CCC CAC (SEQ ID NO:119).

The original pMIN.0 minigene construction was carried out using eight overlapping oligos averaging approximately 70 nucleotides in length, which were synthesized and HPLC purified by Operon Technologies Inc. Each oligo overlapped its neighbor by 15 nucleotides, and the final multi-epitope minigene was assembled by extending the overlapping oligos in three sets of reactions using PCR (Ho et al., Gene 77:51-59 (1989).

For the first PCR reaction, 5 μg of each of two oligos were annealed and extended: 1+2, 3+4, 5+6, and 7+8 were combined in 100 μl reactions containing 0.25 mM each dNTP and 2.5 units of Pfu polymerase in Pfu polymerase buffer containing 10 mM KCl, 10 mM (NH₄)₂SO₄, 20 mM Tris-chloride, pH 8.75, 2 mM MgSO₄, 0.1% TRITON X-100 and 100 mg/ml BSA. A Perkin/Elmer 9600 PCR machine was used and the annealing temperature used was 5° C. below the lowest calculated Tm of each primer pair. The full length dimer products were gel-purified, and two reactions containing the product of 1-2 and 3-4, and the product of 5-6 and 7-8 were mixed, annealed and extended for 10 cycles. Half of the two reactions were then mixed, and 5 cycles of annealing and extension carried out before flanking primers were added to amplify the full length product for 25 additional cycles. The full length product was gel purified and cloned into pCR-blunt (Invitrogen) and individual clones were screened by sequencing. The Min insert was isolated as an NheI-KpnI fragment and cloned into the same sites of pcDNA3.1(−)/Myc-His A (Invitrogen) for expression. The Min protein contains the Myc and His antibody epitope tags at its carboxyl-terminal end.

For all the PCR reactions described, a total of 30 cycles were performed using Pfu polymerase and the following conditions: 95° C. for 15 seconds, annealing temperature for 30 seconds, 72° C. for one minute. The annealing temperature used was 5° C. below the lowest calculated Tm of each primer pair.

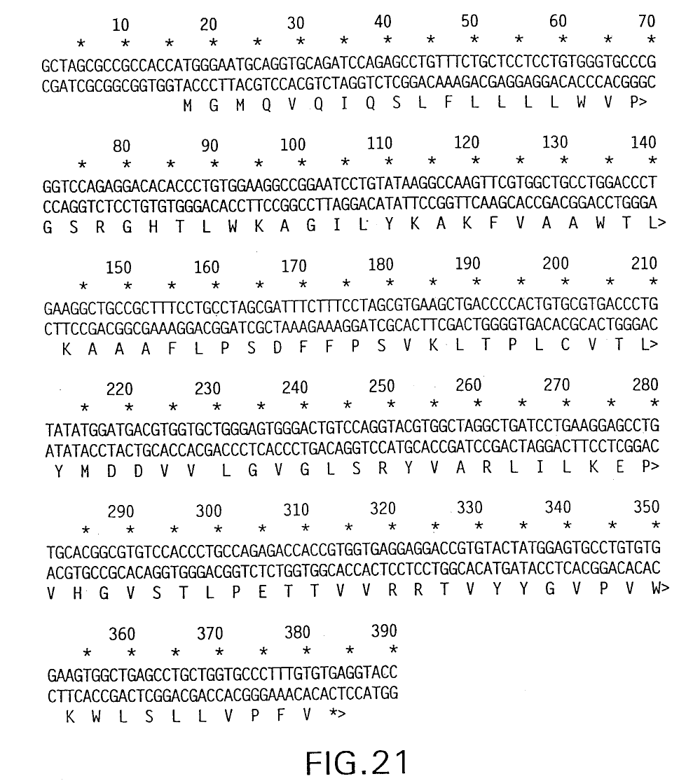

Three changes to pMIN.0 were made to produce pMIN.1 (FIG. 21; SEQ ID NO:37, also referred to as pMIN-AOS). The mouse ova epitope was removed, the position 9 alanine anchor residue (#547) of HBV pol 551-560 was converted to a valine which increased the in vitro binding affinity 40-fold, and a translational stop codon was introduced at the end of the multi-epitope coding sequence. The changes were made by amplifying two overlapping fragments and combining them to yield the full length product.

The first reaction used the 5′ pcDNA vector primer T7 and the primer Min-ovaR (nucleotides 247-218) TGGACAGTCCCACTCCCAGCACCACGTCAT (SEQ ID NO:120). The 3′ half was amplified with the primers: Min-ovaF (nucleotides 228-257) GCTGGGAGTGGGACTGTCCAGGTACGTGGC (SEQ ID NO:121) and Min-StopR (nucleotides 390-361) GGTACCTCACACAAAGGGCACCAGCAGGC (SEQ ID NO:122)

The two fragments were gel purified, mixed, denatured, annealed, and filled in with five cycles of PCR. The full length fragment was amplified with the flanking primers T7 and Min-Stop for 25 more cycles. The product was gel purified, digested with NheI and KpnI and cloned into pcDNA3.1 for sequencing and expression. The insert from pMin.1 was isolated as an NheI-KpnI fragment and cloned into pEP2 to make pEP2-AOS.

### Example II

### Assay for T Helper Cell Activation

This example shows methods for assaying T helper cell activity. One method for assaying T helper cell activity uses spleen cells of an immunized organism. Briefly, a spleen cell pellet is suspended with 2-3 ml of red blood cell lysis buffer containing 8.3 g/liter ammonium chloride in 0.001 M Tris-HCl, pH 7.5. The cells are incubated in lysis buffer for 3-5 min at room temperature with occasional vortexing. An excess volume of 50 ml of R10 medium is added to the cells, and the cells are pelleted. The cells are resuspended and pelleted one or two more times in R2 medium or R10 medium.

The cell pellet is suspended in R10 medium and counted. If the cell suspension is aggregated, the aggregates are removed by filtration or by allowing the aggregates to settle by gravity. The cell concentration is brought to 10⁷/ml, and 100 μl of spleen cells are added to 96 well flat bottom plates.

Dilutions of the appropriate peptide, such as pan DR epitope (SEQ ID NO:145), are prepared in R10 medium at 100, 10, 1, 0.1 and 0.01 μg/ml, and 100 μl of peptide are added to duplicate or triplicate wells of spleen cells. The final peptide concentration is 50, 5, 0.5, 0.05 and 0.005 μg/ml. Control wells receive 100 μl R10 medium.

The plates are incubated for 3 days at 37° C. After 3 days, 20,L of 50 μCi/ml ³H-thymidine is added per well. Cells are incubated for 18-24 hours and then harvested onto glass fiber filters. The incorporation of ³H-thymidine into DNA of proliferating cells is measured in a beta counter.

A second assay for T helper cell activity uses peripheral blood mononuclear cells (PBMC) that are stimulated in vitro as described in Alexander et al., supra and Sette (WO 95/07,707), as adapted from Manca et al., J. Immunol. 146:1964-1971 (1991), which is incorporated herein by reference. Briefly, PBMC are collected from healthy donors and purified over Ficoll-Plaque (Pharmacia Biotech; Piscataway, N.J.). PBMC are plated in a 24 well tissue culture plate at 4×10⁶ cells/ml. Peptides are added at a final concentration of 10 μg/ml. Cultures are incubated at 37° C. in 5% CO₂.

On day 4, recombinant interleukin-2 (IL-2) is added at a final concentration of 10 ng/ml. Cultures are fed every 3 days by aspirating 1 ml of medium and replacing with fresh medium containing IL-2. Two additional stimulations of the T cells with antigen are performed on approximately days 14 and 28. The T cells (3×10⁵/well) are stimulated with peptide (10 μg/ml) using autologous PBMC cells (2×10⁶ irradiated cells/well) (irradiated with 7500 rads) as antigen-presenting cells in a total of three wells of a 24 well tissue culture plate. In addition, on day 14 and 28, T cell proliferative responses are determined under the following conditions: 2×10⁴ T cells/well; 1×10⁵ irradiated PBMC/well as antigen-presenting cells; peptide concentration varying between 0.01 and 101 g/ml final concentration. The proliferation of the T cells is measured 3 days later by the addition of 3H-thymidine (1 μCi/well) 18 hr prior to harvesting the cells. Cells are harvested onto glass filters and ³H-thymidine incorporation is measured in a beta plate counter. These results demonstrate methods for assaying T helper cell activity by measuring ³H-thymidine incorporation.

### Example III

### Assay for Cytotoxic T Lymphocyte Response

This example shows a method for assaying cytotoxic T lymphocyte (CTL) activity. A CTL response is measured essentially as described previously (Vitiello et al., Eur J. Immunol. 27:671-678 (1997), which is incorporated herein by reference). Briefly, after approximately 10-35 days following DNA immunization, splenocytes from an animal are isolated and co-cultured at 37° C. with syngeneic, irradiated (3000 rad) peptide-coated LPS blasts (1×10⁶ to 1.5×10⁶ cells/ml) in 10 ml R10 in T25 flasks. LPS blasts are obtained by activating splenocytes (1×10⁶ to 1.5×10⁶ cells/ml) with 25 μg/ml lipopolysaccharides (LPS) (Sigma cat. no. L-2387; St. Louis, Mo.) and 7 μg/ml dextran sulfate (Pharmacia Biotech) in 30 ml R10 medium in T75 flasks for 3 days at 37° C. The lymphoblasts are then resuspended at a concentration of 2.5×10⁷ to 3.0×10⁷/ml, irradiated (3000 rad), and coated with the appropriate peptides (100 μg/ml) for 1 h at 37° C. Cells are washed once, resuspended in R10 medium at the desired concentration and added to the responder cell preparation. Cultures are assayed for cytolytic activity on day 7 in a ⁵¹Cr-release assay.

For the ⁵¹Cr-release assay, target cells are labeled for 90 min at 37° C. with 150 μl sodium ⁵¹chromate (51 Cr) (New England Nuclear; Wilmington Del.), washed three times and resuspended at the appropriate concentration in R10 medium. For the assay, 104 target cells are incubated in the presence of different concentrations of effector cells in a final volume of 200 μl in U-bottom 96 well plates in the presence or absence of 10 μg/ml peptide. Supernatants are removed after 6 h at 37° C., and the percent specific lysis is determined by the formula: percent specific lysis=100×(experimental release−spontaneous release)/(maximum release−spontaneous release). To facilitate comparison of responses from different experiments, the percent release data is transformed to lytic units 30 per 10⁶ cells (LU30/10⁶), with 1 LU30 defined as the number of effector cells required to induce 30% lysis of 10 target cells in a 6 h assay. LU values represent the LU30/10⁶ obtained in the presence of peptide minus LU30/10⁶ in the absence of peptide. These results demonstrate methods for assaying CTL activity by measuring ⁵¹Cr release from cells.

### Example IV

### T Cell Proliferation in Mice Immunized with Expression Vectors Encoding MHC Class II Epitopes and MHC Class II Targeting Sequences

This example demonstrates that expression vectors encoding MHC class II epitopes and MHC class II targeting sequences are effective at activating T cells.

The constructs used in the T cell proliferation assay are described in Example I and were cloned into the vector pEP2, a CMV driven expression vector. The peptides used for T cell in vitro stimulation are: Ova 323-339, ISQAVHAAHAEINEAGR (SEQ ID NO:48); HBVcore128, TPPAYRPPNAPILF (SEQ ID NO:124); HBVeny182, FFLLTRILTIPQSLD (SEQ ID NO:50); and PADRE, AKFVAAWTLKAAA (SEQ ID NO:52).

T cell proliferation was assayed essentially as described in Example II. Briefly, 12 to 16 week old B6D2 F1 mice (2 mice per construct) were injected with 100 μg of the indicated expression vector (50 μg per leg) in the anterior tibialis muscle. After eleven days, spleens were collected from the mice and separated into a single cell suspension by Dounce homogenization. The splenocytes were counted and one million splenocytes were plated per well in a 96-well plate. Each sample was done in triplicate. Ten μg/ml of the corresponding peptide encoded by the respective expression vectors was added to each well. One well contained splenocytes without peptide added for a negative control. Cells were cultured at 37° C., 5% CO₂ for three days.

After three days, one μCi of ³H-thymidine was added to each well. After 18 hours at 37° C., the cells were harvested onto glass filters and ³H incorporation was measured on an LKB β plate counter. The results of the T cell proliferation assay are shown in Table 9. Antigenspecific T cell proliferation is presented as the stimulation index (SI); this is defined as the ratio of the average ³H-thymidine incorporation in the presence of antigen divided by the ³H-thymidine incorporation in the absence of antigen.

The immunogen “PADRE+IFA” is a positive control where the PADRE peptide in incomplete Freund's adjuvant was injected into the mice and compared to the response seen by injecting the MHC class II epitope constructs containing a PADRE sequence. As shown in Table 9, most of the expression vectors tested were effective at activating T cell proliferation in response to the addition of PADRE peptide. The activity of several of the expression vectors was comparable to that seen with immunization with the PADRE peptide in incomplete Freund's adjuvant. The expression vectors containing both MHC class I and MHC class II epitopes, pEP2-AOS and pcDNA-AOS, were also effective at activating T cell proliferation in response to the addition of PADRE peptide.

These results show that expression vectors encoding MHC class II epitopes fused to a MHC class II targeting sequence is effective at activating T cell proliferation and are useful for stimulating an immune response.

### Example V

### In Vivo Assay Using Transgenic Mice

A. Materials and Methods

Peptides were synthesized according to standard F-moc solid phase synthesis methods which have been previously described (Ruppert et al., Cell 74:929 (1993); Sette et al., Mol. Immunol. 31:813 (1994)). Peptide purity was determined by analytical reverse-phase HPLC and purity was routinely >95%. Synthesis and purification of the Theradigm-HBV lipopeptide vaccine is described in (Vitiello et al., J. Clin. Invest. 95:341 (1995)).

Mice

HLA-A2.1 transgenic mice used in this study were the F1 generation derived by crossing transgenic mice expressing a chimeric gene consisting of the α1, α2 domains of HLA-A2.1 and α3 domain of H-2K^{b} with SJL/J mice (Jackson Laboratory, Bar Harbor, Me.). This strain will be referred to hereafter as HLA-A2.1/K^{b}-H-2^{bxs}. The parental HLA-A2.1/K^{b} transgenic strain was generated on a C57BL/6 background using the transgene and methods described in (Vitiello et al., J. Exp. Med. 173:1007 (1991)). HLA-A11/K^{b} transgenic mice used in the current study were identical to those described in (Alexander et al., J. Immunol. 159:4753 (1997)).

Cell lines, MHC Purification, and Peptide Binding Assay

Target cells for peptide-specific cytotoxicity assays were Jurkat cells transfected with the HLA-A2.1/K^{b} chimeric gene (Vitiello et al., J. Exp. Med. 173:1007 (1991)) and 0.221 tumor cells transfected with HLA-A11/K^{b} (Alexander et al., J. Immunol. 159:4753 (1997)). b

To measure presentation of endogenously processed epitopes, Jurkat-A2.1/K^{b} cells were transfected with the pMin.1 or pMin.2-GFP minigenes then tested in a cytotoxicity assay against epitope-specific CTL lines. For transfection, Jurkat-A2.1/K^{b} cells were resuspended at 10⁷ cells/ml and 30 μg of DNA was added to 600 μl of cell suspension. After electroporating cells in a 0.4 cm cuvette at 0.25 kV, 960 μFd, cells were incubated on ice for 10 min then cultured for 2 d in RPMI culture medium. Cells were then cultured in medium containing 200 U/ml hygromycin B (Calbiochem, San Diego Calif.) to select for stable transfectants. FACS was used to enrich the fraction of green fluorescent protein (GFP)-expressing cells from 15% to 60% (data not shown).

Methods for measuring the quantitative binding of peptides to purified HLA-A2.1 and −A11 molecules is described in Ruppert et al., Cell 74:929 (1993); Sette et al., Mol. Immunol. 31:813 (1994); Alexander et al., J. Immunol. 159:4753 (1997).

All tumor cell lines and splenic CTLs from primed mice were grown in culture medium (CM) that consisted of RPMI 1640 medium with Hepes (Life Technologies, Grand Island, N.Y.) supplemented with 10% FBS, 4 mM L-glutamine, 5×10⁻⁵M 2-ME, 0.5 mM sodium pyruvate, 100 μg/ml streptomycin, and 100 U/ml penicillin.

Construction of Minigene Multi-Epitope DNA Plasmids

pMIN.0 and pMIN.1 (i.e., pMIN-AOS) were constructed as described above and in U.S. S No. 60/085,751.

pMin.1-No PADRE and pMin.1-Anchor. pMin.1 was amplified using two overlapping fragments which was then combined to yield the full length product. The first reaction used the 5′ pcDNA vector primer T7 and either primer ATCGCTAGGCAGGAACTTATACAGGATTCC (SEQ ID NO:126) for pMin.1-No PADRE or TGGACAGTCCGGCTCCCAGCACCACGT (SEQ ID NO:127) for pMin.1-Anchor. The 3′ half was amplified with the primers TTCCTGCCTAGCGATTTC (SEQ ID NO:128) (No PADRE) or GCTGGGAGCCGGACTGTCCAGGTACGT (SEQ ID NO:129) (Anchor) and Min-StopR. The two fragments generated from amplifying the 5′ and 3′ ends were gel purified, mixed, denatured, annealed, and filled in with five cycles of PCR. The full length fragment was further amplified with the flanking primers T7 and Min-StopR for 25 more cycles.

Min.1-No Sig. The Ig signal sequence was deleted from pMin.1 by PCR on with primer GCTAGCGCCGCCACCATGCACACCCTGTGGAAGGC CGGAATC (SEQ ID NO:130) and pcDNA rev (Invitrogen) primers. The product was cloned into pCR-blunt and sequenced.

pMin.1-Switch. Three overlapping fragments were amplified from pMin.1, combined, and extended. The 5′ fragment was amplified with the vector primer T7 and primer GGGCACCAGCAGGCTCAGCCACACTCCCAGCACCACGTC (SEQ ID NO:131). The second overlapping fragment was amplified with primers AGCCTGCTGGTGCCCTTTGTGATCCTGAAGGAGCCTGTGC (SEQ ID NO:132) and AGCCACGTACCTGGACAGTCCCTTCCACACAGGCACTCCAT (SEQ ID NO:133). Primer TGTCCAGGTACGTGGCTAGGCTGTGAGGTACC (SEQ ID NO:134) and the vector primer pcDNA rev (Invitrogen) were used to amplify the third (3′) fragment. Fragments 1, 2, and 3 were amplified and gel purified. Fragments 2 and 3 were mixed, annealed, amplified, and gel purified. Fragment 1 was combined with the product of 2 and 3, and extended, gel purified and cloned into pcDNA3.1 for expression.

pMin.2-GFP. The signal sequence was deleted from pMin.0 by PCR amplification with Min.0-No Sig-5′ GCTAGCGCCGCCACCATGCACACCCTGTGGAAGGCCGGAATC (SEQ ID NO:135) plus pcDNA rev (Invitrogen) primers. The product was cloned into pCR-blunt and sequenced. The insert containing the open reading frame of the signal sequence-deleted multi-epitope construct was cut out with NheI plus HindIII and ligated into the same sites of pEGFPN1 (Clontech). This construct fuses the coding region of the signal-deleted pMin.0 construct to the N-terminus of green fluorescent protein (GFP).

Immunization of Mice

For DNA immunization, mice were pretreated by injecting 50 μl of 10 μM cardiotoxin (Sigma Chem. Co., #C9759) bilaterally into the tibialis anterior muscle. Four or five days later, 100 μg of DNA diluted in PBS were injected in the same muscle.

Theradigm-HBV lipopeptide (10 mg/ml in DMSO) that was stored at −20° C., was thawed for 10 min at 45° C. before being diluted 1:10 (v/v) with room temperature PBS. Immediately upon addition of PBS, the lipopeptide suspension was vortexed vigorously and 100 μl was injected s.c. at the tail base (100 μg/mouse).

Immunogenicity of individual CTL epitopes was tested by mixing each CTL epitope (50 μg/mouse) with the HBV core 128-140 peptide (TPPAYRPPNAPIL (SEQ ID NO:49), 140 μg/mouse) which served to induce I-A^{b}-restricted Th cells. The peptide cocktail was then emuslifed in incomplete Freund's adjuvant (Sigma Chem. Co.) and 100 μl of peptide emulsion was injected s.c. at the tail base.

In Vitro CTL Cultures and Cytotoxicity Assays

Eleven to 14 days after immunization, animals were sacrificed and a single cell suspension of splenocytes prepared. Splenocytes from cDNA-primed animals were stimulated in vitro with each of the peptide epitopes represented in the minigene. Splenocytes (2.5-3.0×10⁷/flask) were cultured in upright 25 cm² flasks in the presence of 10 μg/ml peptide and 10⁷ irradiated spleen cells that had been activated for 3 days with LPS (25 μg/ml) and dextran sulfate (7 μg/ml). Triplicate cultures were stimulated with each epitope. Five days later, cultures were fed with fresh CM. After 10 d of in vitro culture, 2-4×10⁶ CTLs from each flask were restimulated with 10⁷ LPS/dextran sulfate-activated splenocytes treated with 100 μg/ml peptide for 60-75 min at 37° C., then irradiated 3500 rads. CTLs were restimulated in 6-well plates in 8 ml of cytokine-free CM. Eighteen hr later, cultures received cytokines contained in con A-activated splenocyte supernatant (10-15% final concentration, v/v) and were fed or expanded on the third day with CM containing 10-15% cytokine supernate. Five days after restimulation, CTL activity of each culture was measured by incubating varying numbers of CTLs with 10⁴ ⁵¹Cr-labelled target cells in the presence or absence of peptide. To decrease nonspecific cytotoxicity from NK cells, YAC-1 cells (ATCC) were also added at a YAC-1: ⁵¹Cr-labeled target cell ratio of 20:1. CTL activity against the HBV Pol 551 epitope was measured by stimulating DNA-primed splenocytes in vitro with the native A-containing peptide and testing for cytotoxic activity against the same peptide.

To more readily compare responses, the standard E:T ratio vs % cytotoxicity data curves were converted into LU per 10⁶ effector cells with one LU defined as the lytic activity required to achieve 30% lysis of target cells at a 100:1 E:T ratio. Specific CTL activity (ALU) was calculated by subtracting the LU value obtained in the absence of peptide from the LU value obtained with peptide. A given culture was scored positive for CTL induction if all of the following criteria were met: 1) ALU >2; 2) LU(+peptide)÷LU(−peptide)>3; and 3) a >10% difference in % cytotoxicity tested with and without peptide at the two highest E:T ratios (starting E:T ratios were routinely between 25-50:1).

CTL lines were generated from pMin.1-primed splenocytes through repeated weekly stimulations of CTLs with peptide-treated LPS/DxS-activated splenocytes using the 6-well culture conditions described above with the exception that CTLs were expanded in cytokine-containing CM as necessary during the seven day stimulation period.

Cytokine Assay

To measure IFN-γ production in response to minigene-transfected target cells, 4×10⁴ CTLs were cultured with an equivalent number of minigene-transfected Jurkat-A2.1/K^{b} cells in 96-well flat bottom plates. After overnight incubation at 37° C., culture supernatant from each well was collected and assayed for IFN-γ concentration using a sandwich ELISA. Immulon II microtiter wells (Dynatech, Boston, Mass.) were coated overnight at 4° C. with 0.2 μg of anti-mouse IFN-γ capture Ab, R4-6A2 (Pharmingen). After washing wells with PBS/0.1% Tween-20 and blocking with 1% BSA, Ab-coated wells were incubated with culture supernate samples for 2 hr at room temperature. A secondary anti-IFN-γ Ab, XMG1.2 (Pharmingen), was added to wells and allowed to incubate for 2 hr at room temperature. Wells were then developed by incubations with Avidin-DH and finally with biotinylated horseradish peroxidase H (Vectastain ABC kit, Vector Labs, Burlingame, Calif.) and TMB peroxidase substrate (Kirkegaard and Perry Labs, Gaithersberg, Md.). The amount of cytokine present in each sample was calculated using a rIFN-γ standard (Pharmingen).

B. Results

Selection of Epitopes and Minigene Construct Design

In the first series of experiments, the issue was whether a balanced multispecific CTL response could be induced by simple minigene cDNA constructs that encode several dominant HLA class I-restricted epitopes. Accordingly, nine CTL epitopes were chosen on the basis of their relevance in CTL immunity during HBV and HIV infection in humans, their sequence conservancy among viral subtypes, and their class I MHC binding affinity (Table 10). Of these nine epitopes, six are restricted by HLA-A2.1 and three showed HLA-A11-restriction. One epitope, HBV Pol 551, was studied in two alternative forms: either the wild type sequence or an analog (HBV Pol 551-V) engineered for higher binding affinity.

As referenced in Table 10, several independent laboratories have reported that these epitopes are part of the dominant CTL response during HBV or HIV infection. All of the epitopes considered showed greater than 75% conservancy in primary amino acid sequence among the different HBV subtypes and HIV clades. The MHC binding affinity of the peptides was also considered in selection of the epitopes. These experiment addressed the feasibility of immunizing with epitopes possessing a wide range of affinities and, as shown in Table 10, the six HBV and three HIV HLA-restricted epitopes covered a spectrum of MHC binding affinities spanning over two orders of magnitude, with IC50% concentrations ranging from 3 nM to 200 nM.

The immunogenicity of the six A2.1- and three A11-restricted CTL epitopes in transgenic mice was verified by co-immunization with a helper T cell peptide in an IFA formulation. All of the epitopes induced significant CTL responses in the 5 to 73 ΔLU range (Table 10). As mentioned above, to improve the MHC binding and immunogenicity of HBV Pol 551, the C-terminal A residue of this epitope was substituted with V resulting in a dramatic 40-fold increase in binding affinity to HLA-A2.1 (Table 10). While the parental sequence was weakly or nonimmunogenic in HLA transgenic mice, the HBV Pol 551-V analog induced significant levels of CTL activity when administered in IFA (Table 10). On the basis of these results, the V analog of the HBV Pol 551 epitope was selected for the initial minigene construct. In all of the experiments reported herein, CTL responses were measured with target cells coated with the native HBV Pol 551 epitope, irrespective of whether the V analog or native epitope was utilized for immunization.

Finally, since previous studies indicated that induction of T cell help significantly improved the magnitude and duration of CTL responses (Vitiello et al., J. Clin. Invest. 95:341 (1995); Livingston et al., J. Immunol. 159:1383 (1997)), the universal Th cell epitope PADRE was also incorporated into the minigene. PADRE has been shown previously to have high MHC binding affinity to a wide range of mouse and human MHC class II haplotypes (Alexander et al., Immunity 1:751 (1994)). In particular, it has been previously shown that PADRE is highly immunogenic in H-2b mice that are used in the current study (Alexander et al., Immunity 1:751 (1994)).

pMin.1, the prototype cDNA minigene construct encoding nine CTL epitopes and PADRE, was synthesized and subcloned into the pcDNA3.1 vector. The position of each of the nine epitopes in the minigene was optimized to avoid junctional mouse H-2b and HLA-A2.1 class I MHC epitopes. The mouse Ig K signal sequence was also included at the 5′ end of the construct to facilitate processing of the CTL epitopes in the endoplasmic reticulum (ER) as reported by others (Anderson et al., J. Exp. Med. 174:489 (1991)). To avoid further conformational structure in the translated polypeptide gene product that may affect processing of the CTL epitopes, an ATG stop codon was introduced at the 3′ end of the minigene construct upstream of the coding region for c-myc and poly-his epitopes in the pcDNA3.1 vector.

Immunogenicity of pMin.1 in Transgenic Mice

To assess the capacity of the pMin.1 minigene construct to induce CTLs in vivo, HLA-A2.1/K^{b}-H-2^{bxs} transgenic mice were immunized intramuscularly with 100 μg of naked cDNA. As a means of comparing the level of CTLs induced by cDNA immunization, a control group of animals was also immunized with Theradigm-HBV, a palmitolyated lipopeptide consisting of the HBV Core 18 CTL epitope linked to the tetanus toxin 830-843 Th cell epitope.

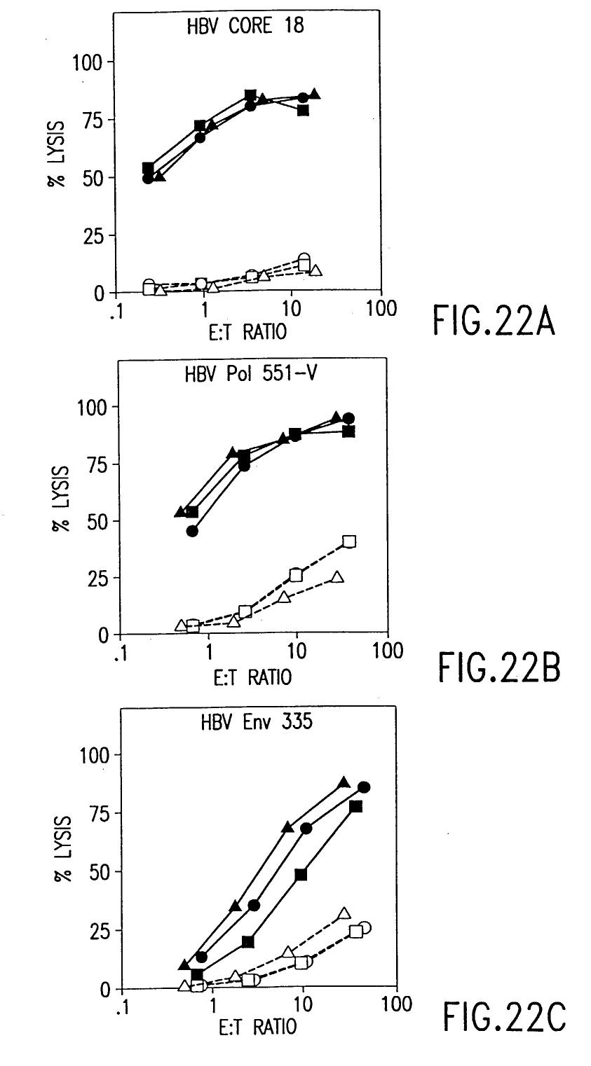

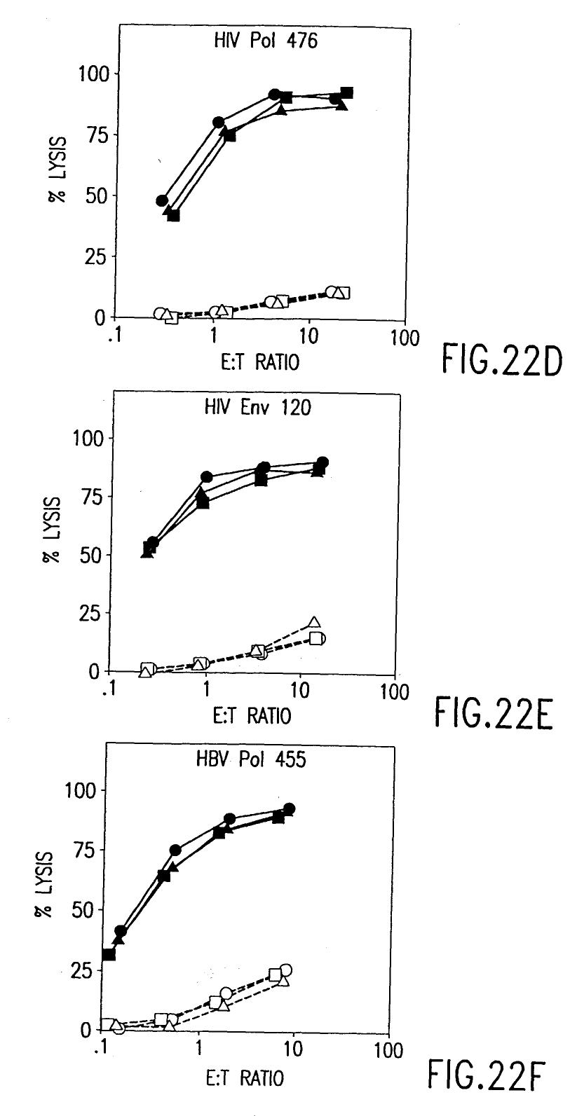

Splenocytes from immunized animals were stimulated twice with each of the peptide epitopes encoded in the minigene, then assayed for peptide-specific cytotoxic activity in a ⁵¹Cr release assay. A representative panel of CTL responses of pMin.1-primed splenocytes, shown in FIG. 22, clearly indicates that significant levels of CTL induction were generated by minigene immunization. The majority of the cultures stimulated with the different epitopes exceeded 50% specific lysis of target cells at an E:T ratio of 1:1. The results of four independent experiments, compiled in Table 11, indicate that the pMin.1 construct is indeed highly immunogenic in HLA-A_{2.1}/K^{b}-H-₂^{bxs} transgenic mice, inducing a broad CTL response directed against each of its six A2.1-restricted epitopes.

To more conveniently compare levels of CTL induction among the different epitopes, the % cytotoxicity values for each splenocyte culture was converted to ALU and the mean ALU of CTL activity in positive cultures for each epitope was determined (see Example V, materials and methods, for positive criteria). The data, expressed in this manner in Table 11, confirms the breadth of CTL induction elicited by pMin.1 immunization since extremely high CTL responses, ranging between 50 to 700 ALU, were observed against the six A2.1-restricted epitopes. More significantly, the responses of several hundred ALU observed for five of the six epitopes approached or exceeded that of the Theradigm-HBV lipopeptide, a vaccine formulation known for its high CTL-inducing potency (Vitiello et al., J. Clin. Invest. 95:341 (1995); Livingston et al., J. Immunol. 159:1383 (1997)). The HBV Env 335 epitope was the only epitope showing a lower mean ALU response compared to lipopeptide (Table 11, 44 vs 349 ALU).

Processing of Minigene Epitopes by Transfected Cells

The decreased CTL response observed against HBV Env 335 was somewhat unexpected since this epitope had good A2.1 binding affinity (IC50%, 5 nM) and was also immunogenic when administered in IFA. The lower response may be due, at least in part, to the inefficient processing of this epitope from the minigene polypeptide by antigen presenting cells following in vivo cDNA immunization. To address this possibility, Jurkat-A2.1/K^{b} tumor cells were transfected with pMin.1 cDNA and the presentation of the HBV Env 335 epitope by transfected cells was compared to more immunogenic A2.1-restricted epitopes using specific CTL lines. Epitope presentation was also studied using tumor cells transfected with a control cDNA construct, pMin.2-GFP, that encoded a similar multi-epitope minigene fused with GFP which allows detection of minigene expression in transfected cells by FACS.

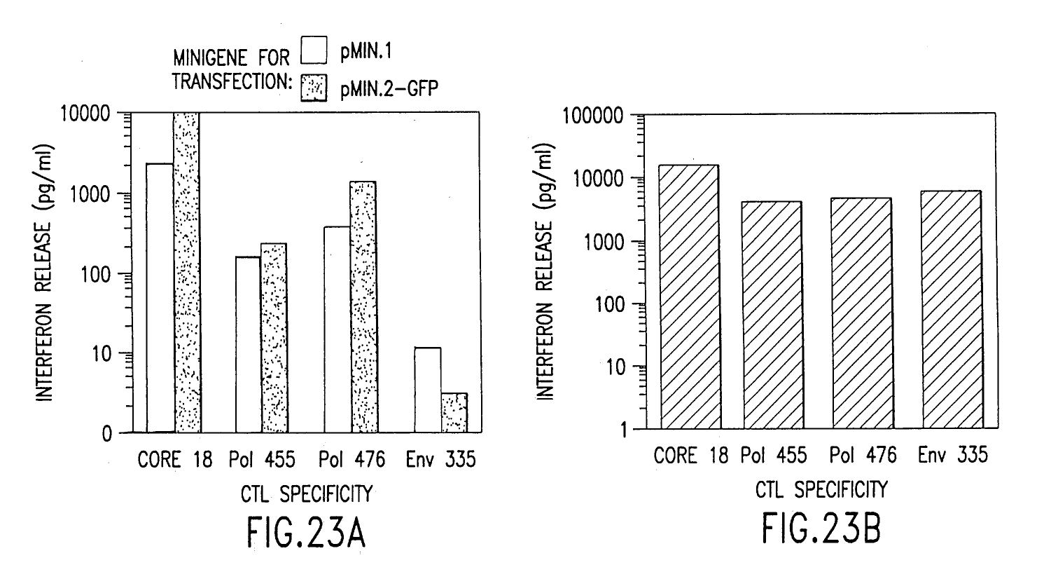

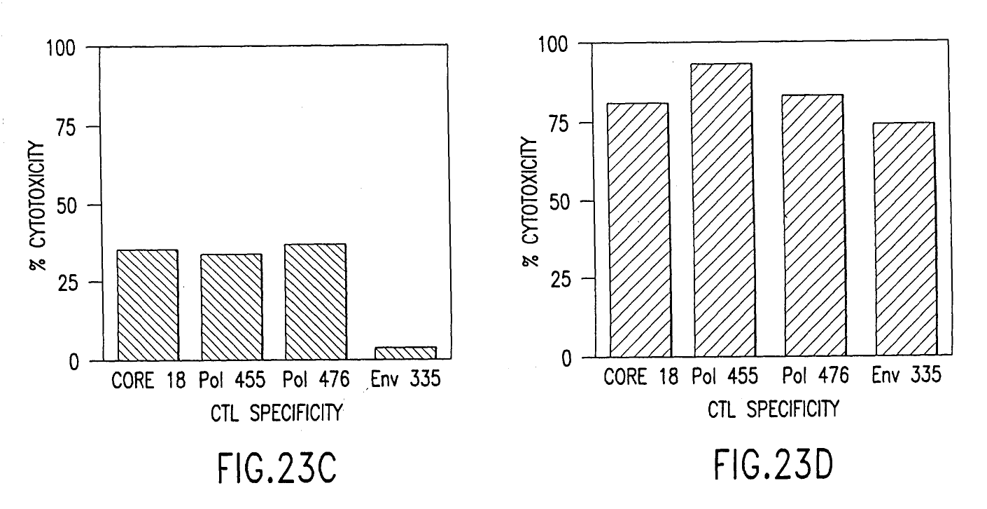

Epitope presentation of the transfected Jurkat cells was analyzed using specific CTL lines, with cytotoxicity or IFN-γ production serving as a read-out. It was found that the levels of CTL response correlated directly with the in vivo immunogenicity of the epitopes. Highly immunogenic epitopes in vivo, such as HBV Core 18, HIV Pol 476, and HBV Pol 455, were efficiently presented to CTL lines by pMin.1- or pMin.2-GFP-transfected cells as measured by IFN-γ production (FIG. 23A, >100 pg/ml for each epitope) or cytotoxic activity (FIG. 23C, >30% specific lysis). In contrast to these high levels of in vitro activity, the stimulation of the HBV Env 335-specific CTL line against both populations of transfected cells resulted in less than 12 pg/ml IFN-γ and 3% specific lysis. Although the HBV Env 335-specific CTL line did not recognize the naturally processed epitope efficiently, this line did show an equivalent response to peptide-loaded target cells, as compared to CTL lines specific for the other epitopes (FIG. 23B, D). Collectively, these results suggest that a processing and/or presentation defect associated with the HBV Env 335 epitope that may contribute to its diminished immunogencity in vivo.

Effect of the Helper T Cell Epitope PADRE on Minigene Immunogenicity

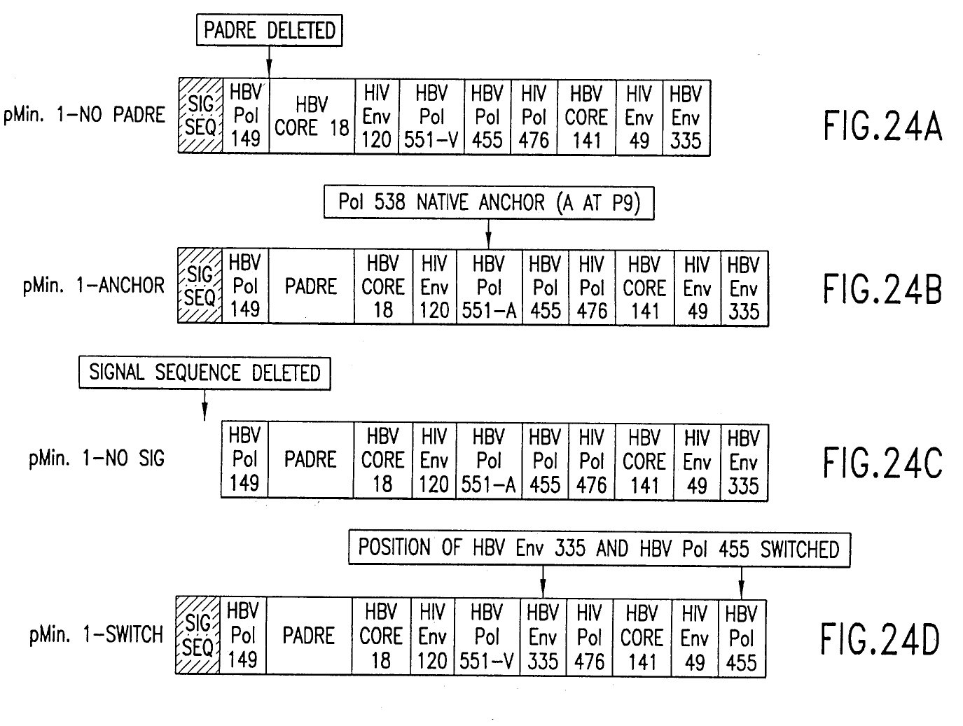

Having obtained a broad and balanced CTL response in transgenic mice immunized with a minigene cDNA encoding multiple HLA-A2.1-restricted epitopes, next possible variables were examined that could influence the immunogenicity of the prototype construct. This type of analysis could lead to rational and rapid optimization of future constructs. More specifically, a cDNA construct based on the pMin.1 prototype was synthesized in which the PADRE epitope was deleted to examine the contribution of T cell help in minigene immunogenicity (FIG. 24A).

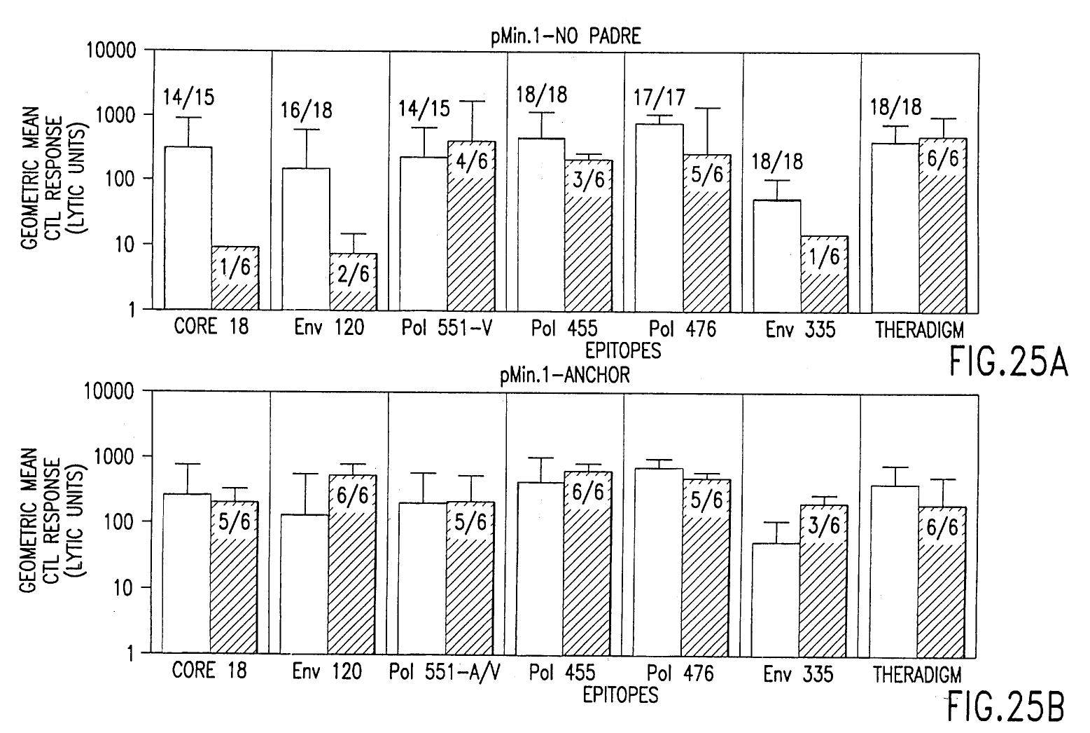

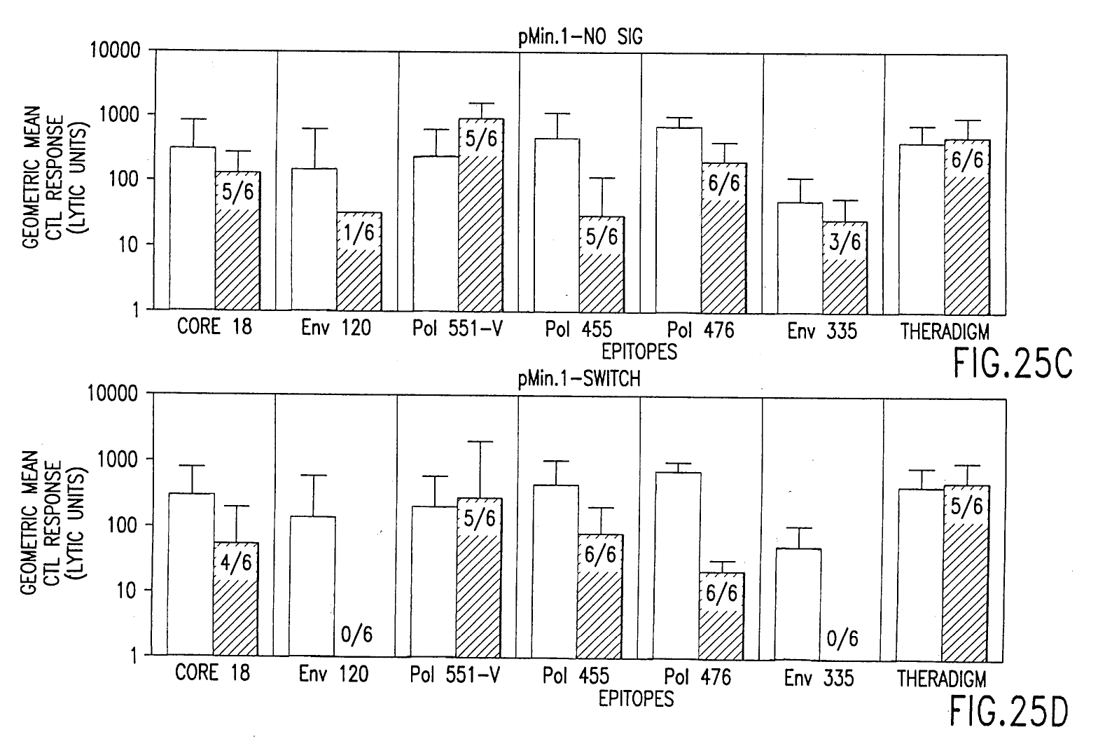

The results of the immunogenicity analysis indicated that deletion of the PADRE Th cell epitope resulted in significant decreases in the frequency of specific CTL precursors against four of the minigene epitopes (HBV Core 18, HIV Env 120, HBV Pol 455, and HBV Env 335) as indicated by the 17 to 50% CTL-positive cultures observed against these epitopes compared to the 90-100% frequency in animals immunized with the prototype pMin.1 construct (FIG. 25). Moreover, for two of the epitopes, HBV Core 18 and HIV Env 120, the magnitude of response in positive cultures induced by pMin.1-No PADRE was 20- to 30-fold less than that of the pMin.1 construct (FIG. 25A).

Effect of Modulation of MHC Binding Affinity on Epitope Immunogenicity

Next a construct was synthesized in which the V anchor residue in HBV Pol 551 was replaced with alanine, the native residue, to address the effect of decreasing MHC binding on epitope immunogenicity (FIG. 24B).

Unlike deletion of the Th cell epitope, decreasing the MHC binding capacity of the HBV Pol 551 epitope by 40-fold through modification of the anchor residue did not appear to affect epitope immunogenicity (FIG. 25B). The CTL response against the HBV Pol 551 epitope, as well as to the other epitopes, measured either by LU or frequency of CTL-positive cultures, was very similar between the constructs containing the native A or improved V residue at the MHC binding anchor site. This finding reinforces the notion that minimal epitope minigenes can efficiently deliver epitopes of vastly different MHC binding affinities. Furthermore, this finding is particularly relevant to enhancing epitope immunogenicity via different delivery methods, especially in light of the fact that the wild type HBV Pol 551 epitope was essentially nonimmunogenic when delivered in a less potent IFA emulsion.

Effect of the Signal Sequence on Minigene Construct Immunogenicity

The signal sequence was deleted from the pMin.1 construct, thereby preventing processing of the minigene polypeptide in the ER (FIG. 24C). When the immunogenicity of the pMin.1-No Sig construct was examined, an overall decrease in response was found against four CTL epitopes. Two of these epitopes, HIV Env 120 and HBV Env 335, showed a decrease in frequency of CTL-positive cultures compared to pMin.1 while the remaining epitopes, HBV Pol 455 and HIV Pol 476, showed a 16-fold (from 424 to 27 ALU) and 3-fold decrease (709 to 236 ALU) in magnitude of the mean CTL response, respectively (FIG. 25C). These findings suggest that allowing ER-processing of some of the epitopes encoded in the pMin.1 prototype construct may improve immunogenicity, as compared with constructs that allow only cytoplasmic processing of the same panel of epitopes.

Effect of Epitope Rearrangement and Creation of New Junctional Epitopes

In the final construct tested, the immunogenicity of the HBV Env 335 epitope was analyzed to determine whether it may be influenced by its position at the 3′ terminus of the minigene construct (FIG. 24D). Thus, the position of the Env epitope in the cDNA construct was switched with a more immunogenic epitope, HBV Pol 455, located in the center of the minigene. It should be noted that this modification also created two potentially new epitopes. As shown in FIG. 25D, the transposition of the two epitopes appeared to affect the immunogenicity of not only the transposed epitopes but also more globally of other epitopes. Switching epitopes resulted in obliteration of CTL induction against HBV Env 335 (no positive cultures detected out of six). The CTL response induced by the terminal HBV Pol 455 epitope was also decreased but only slightly (424 vs 78 mean ALU). In addition to the switched epitopes, CTL induction against other epitopes in the pMin.1-Switch construct was also markedly reduced compared to the prototype construct. For example, a CTL response was not observed against the HIV Env 120 epitope and it was significantly diminished against the HBV Core 18 (4 of 6 positive cultures, decrease in mean ALU from 306 to 52) and HBV Pol 476 (decrease in mean ALU from 709 to 20) epitopes (FIG. 25D).

As previously mentioned, it should be noted that switching the two epitopes had created new junctional epitopes. Indeed, in the pMin.1-Switch construct, two new potential CTL epitopes were created from sequences of HBV Env 335-HIV Pol 476 (LLVPFVIL (SEQ ID NO:123), H-2 Kb-restricted) and HBV Env 335-HBV Pol 551 (VLGVWLSLLV (SEQ ID NO:136), HLA-A2.1-restricted) epitopes. Although these junctional epitopes have not been examined to determine whether or not they are indeed immunogenic, this may account for the low immunogenicity of the HBV Env 335 and HIV Pol 476 epitopes. These findings suggest that avoiding junctional epitopes may be important in designing multi-epitope minigenes as is the ability to confirm their immunogenicity in vivo in a biological assay system such as HLA transgenic mice.

Induction of CTLs Against A11 Epitopes Encoded in pMin.1

To further examine the flexibility of the minigene vaccine approach for inducing a broad CTL response against not only multiple epitopes but also against epitopes restricted by different HLA alleles, HLA-A11/K^{b} transgenic mice were immunized to determine whether the three A11 epitopes in the pMin.1 construct were immunogenic for CTLs, as was the case for the A2.1-restricted epitopes in the same construct. As summarized in Table 12, significant CTL induction was observed in a majority of cultures against all three of the HLA-A11-restricted epitopes and the level of CTL immunity induced for the three epitopes, in the range of 40 to 260 ALU, exceeded that of peptides delivered in IFA (Table 10). Thus, nine CTL epitopes of varying HLA restrictions incorporated into a prototype minigene construct all demonstrated significant CTL induction in vivo, confirming that minigene DNA plasmids can serve as means of delivering multiple epitopes, of varying HLA restrictions and MHC binding affinities, to the immune system in an immunogenic fashion and that appropriate transgenic mouse strains can be used to measure DNA construct immunogenicity in vivo.

CTLs were also induced against three A11 epitopes in A11/K^{b} transgenic mice. These responses suggest that minigene delivery of multiple CTL epitopes that confers broad population coverage may be possible in humans and that transgenic animals of appropriate haplotypes may be a useful tools in optimizing the in vivo immunogenicity of minigene DNA. In addition, animals such as monkeys having conserved HLA molecules with cross reactivity to CTL and HTL epitopes recognized by human MHC molecules can be used to determine human immunogenicity of HTL and CTL epitopes (Bertoni et al., J. Immunol.161:4447-4455 (1998)).

This study represents the first description of the use of HLA transgenic mice to quantitate the in vivo immunogenicity of DNA vaccines, by examining response to epitopes restricted by human HLA antigens. In vivo studies are required to address the variables crucial for vaccine development, that are not easily evaluated by in vitro assays, such as route of administration, vaccine formulation, tissue biodistribution, and involvement of primary and secondary lymphoid organs. Because of its simplicity and flexibility, HLA transgenic mice represent an attractive alternative, at least for initial vaccine development studies, compared to more cumbersome and expensive studies in higher animal species, such as nonhuman primates. The in vitro presentation studies described above further supports the use of HLA transgenic mice for screening DNA constructs containing human epitopes inasmuch as a direct correlation between in vivo immunogenicity and in vitro presentation was observed. Finally, strong CTL responses were observed against all six A 2.1 restricted viral epitopes and in three A11 restricted epitopes encoded in the prototype pMin.1 construct. For five of the A 2.1 restricted epitopes, the magnitude of CTL response approximated that observed with the lipopeptide, Theradigm-HBV, that previously was shown to induce strong CTL responses in humans (Vitiello et al., J. Clin. Invest. 95:341 (1995); Livingston et al., J. Immunol. 159:1383 (1997)).

TABLE 1

HBV derived HTL epitopes

| | | | |
|---|---|---|---|
| | | | SEQ ID |
| Peptide | Sequence | Source | NO: |

| | | | |
|---|---|---|---|
| 1298.06 | KQAFTFSPTYKAFLC | HBV POL 661 | 137 |
| F107.03 | LQSLTNLLSSNLSWL | HBV POL 412 | 138 |
| 1280.06 | AGFFLLTRILTIPQS | HBV ENV 180 | 139 |
| 1280.09 | GTSFVYVPSALNPAD | HBV POL 774 | 140 |
| CF-08 | VSFGVWIRTPPAYRPPNAPI | HBV NUC 120 | 141 |
| 27.0280 | GVWIRTPPAYRPPNA | HBV NUC 123 | 142 |
| 1186.25 | SFGVWIRTPPAYRPP | HBV NUC 121 | 143 |
| 27.0281 | RHYLHTLWKAGILYK | HBV POL 145 | 144 |
| F107.04 | PFLLAQFTSAICSVV | HBV POL 523 | 145 |
| 1186.15 | LVPFVQWFVGLSPTV | HBV ENV 339 | 146 |
| 1280.15 | LHLYSHPIILGFRKI | HBV POL 501 | 147 |
| 1298.04 | KQCFRKLPVNRPIDW | HBV POL 615 | 148 |
| 1298.07 | AANWILRGTSFVYVP | HBV POL 764 | 149 |
| 857.02 | PHHTALRQAILCWGELMTLA | HBV CORE 50 | 150 |
| 35.0100 | LCQVFADATPTGWGL | HBV POL 683 | 151 |
| 35.0096 | ESRLVVDFSQFSRGN | HBV POL 387 | 152 |
| 35.0093 | VGPLTVNEKRRLKLI | HBV POL 96 | 153 |
| 1186.18 | NLSWLSLDVSAAFYH | HBV POL 422 | 154 |

TABLE 2

HBV derived CTL epitopes

| | | | | |
|---|---|---|---|---|
| Supertype | Peptide | Sequence | Source | SEQ ID NO: |

| | | | | |
|---|---|---|---|---|
| A2 | 924.07 | FLPSDFFPSV | HBV core 18-27 | 91 |
| | 1013.0102 | WLSLLVPFV | HBVadr-ENV (S Ag 335-343) | 107 |
| | 777.03 | FLLTRILTI | HBV ENV ayw 183 | 155 |
| | 927.15 | ALMPLYACI | HBV ayw pot 642 | 156 |
| | 1168.02 | GLSRYVARL | HBV POL 455 | 99 |
| | 927.11 | FLLSLGIHL | HBV pol 562 | 157 |
| A3 | 1147.16 | HTLWKAGILYK | HBV POL 149 | 88 |
| | 1083.01 | STLPETTVVRR | HBV core 141 | 103 |
| | 1090.11 | SAICSVVRR | HBV pol 531 | 158 |
| | 1090.10 | QAFTFSPTYK | HBV pol 665 | 159 |
| | 1069.16 | NVSIPWTHK | HBV pol 47 | 160 |
| | 1069.20 | LVVDFSQFSR | HBV pol 388 | 161 |
| | 1142.05 | KVGNFTGLY | HBV adr POL 629 | 162 |
| | 1069.15 | TLWKAGILYK | HBV pol 150 | 163 |
| B7 | 1145.04 | IPIPISSWAF | HBV ENV 313 | 164 |
| | 988.05 | LPSDFFPSV | HBV core 19-27 | 165 |
| | 1147.04 | TPARVTGGVF | HBV POL 354 | 166 |
| A2 | 1069.06 | LLVPFVQWFV | HBV env 338-347 | 167 |
| | 1147.13 | FLLAQFTSAI | HBV POL 513 | 168 |
| | 1147.14 | VLLDYQGMLPV | HBV ENV 259 | 169 |
| | 1132.01 | LVPFVQWFV | HBV ENV 339 | 170 |
| | 1069.05 | LLAQFTSAI | HBV pol 504-512 | 171 |
| | 927.42 | NLSWLSLDV | HBV pol 411 | 172 |
| | 927.41 | LLSSNLSWL | HBV pol 992 | 173 |
| | 927.46 | KLHLYSHPI | HBV pol 489 | 174 |
| | 1069.071 | FLLAQFRSA | HBV pol 503 | 175 |
| | 1142.07 | GLLGWSPQA | HBV ENV 62 | 176 |
| | 927.47 | HLYSHPIIL | HBV ayw pol 1076 | 177 |
| | 1069.13 | PLLPIFFCL | HBV env 377-385 | 178 |
| | 1013.1402 | VLQAGFFLL | HBVadr-ENV 177 | 179 |
| | 1090.14 | YMDDVVLGA | HBV pol 538-546 | 95 |
| A3 | 26.0539 | RLVVDFSQFSR | HBV pol 376 | 180 |
| | 26.0535 | GVWIRTPPAYR | HBV X niuc fus 299 | 181 |
| A3 | 26.0153 | SSAGPCALR | HBV X 64 | 182 |
| | 1.0993 | KVFVLGGCR | HBV adr “X” 1548 | 183 |
| | 26.0149 | CALRFTSAR | HBV X 69 | 184 |
| | 26.0023 | VSFGVWIR | HBV x nuc fus 296 185 | |
| | 26.0545 | TLPETTVVRRR | HBV x nuc fus 318 | 186 |
| | 20.0131 | SVVRRAFPH | HBV POL 524 | 187 |
| | 1.0219 | FVLGGCRHK | HBVadr “X” 1550 | 188 |
| | 26.0008 | FTFSPTYK | HBV pol 656 | 189 |
| | 20.0130 | AFTESPTYK | HBV POL 655 | 190 |
| B7 | 1147.05 | FPHCLAFSYM | HBV POL 530 | 191 |
| | 1147.08 | YPALMPLYA | HBV POL 640 | 192 |
| | 1147.06 | LPVCAFSSA | HBV X 58 | 193 |
| | 1147.02 | HPAAMPHLL | HBV POL 429 | 194 |
| | 26.0570 | YPALMPLYACI | HBV pol 640 | 195 |
| | 19.0014 | YPALMPLY | HBV POL 64O | 196 |
| | 1145.08 | FPHCLAFSY | HBV POL 541 | 197 |
| Other | 1090.02 | AYRPPNAPI | HBV NUC 131 | 198 |
| | 1.0519 | DLLDTASALY | HBV adr CORE 419 | 199 |
| | 13.0129 | EYLVSFGVWI | HBV NUC 117 | 200 |
| | 20.0254 | FAAPFTQCGY | HBV POL 631 | 201 |
| | 2.0060 | GYPALMPLY | HBV ALL 1224 | 202 |
| | 1069.04 | HTLWKAGILY | HBV pol 149 | 203 |
| | 1069.08 | ILLLCLIFLL | HBV env 249-258 | 204 |
| | 1.0166 | KVGNFTGLY | HBV adr POL 629 | 162 |
| | 1069.23 | KYTSFPWLL | HBV POL 745 | 205 |
| | 1069.01 | LLDTASALY | HBV core 59 | 26 |
| | 2.0239 | LSLDVSAAFY | HBV ALL 1000 | 207 |
| | 2.0181 | LYSHPIILGF | HBV POL 492 | 208 |
| | 1039.01 | MMWYWGPSLY | HBV 360 | 209 |
| | 2.0126 | MSTTDLEAY | HBV adr 1521 | 210 |
| | 1069.03 | PLDKGIKPYY | HBV pol 124 | 211 |
| | 1090.09 | PTTGRTSLY | HBV pol 808 | 212 |
| | 20.0138 | PWTHKVGNF | HBV POL 51 | 213 |
| | 20.0135 | RWMCLRRFI | HBV ENV 236 | 214 |
| | 20.0269 | RWMCLRRFII | HBV ENV 236 | 215 |
| | 20.0139 | SFCGSPYSW | HBV POL 167 | 216 |
| Other | 1069.02 | SLDVSAAFY | HBV pol 427 | 217 |
| | 20.0136 | SWLSLLVPF | HBV ENV 334 | 218 |
| | 20.0271 | SWPKFAVPNL | HBV POL 392 | 219 |
| | 20.0137 | SWWTSLNFL | HBV ENV 197 | 220 |
| | 2.0173 | SYQHFRKLLL | HBV POL 4 | 221 |
| | 13.0073 | WFHISCLTF | HBV NUC 102 | 222 |
| | 1.0774 | WLWGMDIDPY | HBV adw CORE 416 | 223 |
| | 1039.06 | WMMWYWGPSLY | HBV env 359 | 224 |
| | 924.14 | FLPSDFFPSI | HBv 18-27 i₁₀ var. | 225 |
| | 1090.77 | YMDDVVLGV | HBV pol 538-546 sub | 462 |
| | 941.01 | FLPSDYFPSV | HBc 18-27 analog | 226 |
| | 1083.02 | STLPETYVVRR | HBV core 141-151 analog | 227 |
| | 1145.05 | FPIPSSWAF | HBV ENV 313 analog | 228 |
| | 1145.11 | FPHCLAFSL | HBV POL 541 analog | 229 |
| | 1145.24 | FPHCLAFAL | HBV POL 541 analog | 230 |
| | 1145.06 | IPITSSWAF | HBV ENV 313 analog | 231 |
| | 1145.23 | IPIPMSWAF | HBV ENV 313 analog | 232 |
| | 1145.07 | IPILSSWAF | HBV ENV 313 analog | 233 |
| | 1145.09 | FPVCLAFSY | HBV POL 541 analog | 234 |
| | 1145.10 | FPHCLAFAY | HBV POL 541 analog | 235 |

TABLE 3

HCV derived HTL epitopes

| | | | |
|---|---|---|---|
| Peptide | Sequence | Source | SEQ ID NO: |

| | | | |
|---|---|---|---|
| | AAYAAQGYKVLVLNPSVAATLGFGAY | HCV NS3 1242-1267 | 236 |
| P98.03 | AAYAAQGYKVLVLNPSVAAT | HCV NS3 1242 | 237 |
| P98.04 | GYKVLVLNPSVAATLGFGAY | HCV NS3 1248 | 238 |
| P98.05 | GYKVLVLNPSVAAT | HCV NS3 1248 | 239 |
| 1283.21 | GYKVLVLNPSVAATL | HCV NS3 1253 | 240 |
| 1283.20 | AQGYKVLVLNPSVAA | HCV NS3 1251 | 241 |
| | GEGAVQWMNRLIAFASRGNHVS | HCV NS4 1914-1935 | 242 |
| F134.08 | GEGAVQWMNRLIAFASRGNHV | HCV NS4 1914 | 243 |
| 1283.44 | MNRLIAFASRGNHVS | HCV NS4 1921 | 244 |
| 1283.16 | SKGWRLLAPITAYAQ | HCV NS3 1025 | 245 |
| 1283.55 | GSSYGFQYSPGQRVE | HCV NS5 2641 | 246 |
| F134.05 | NFISGIQYLAGLSTLPGNPA | HCV NS4 1772 | 247 |
| 1283.61 | ASCLRKLGVPPLRVW | HCV NS5 2939 | 248 |
| 1283.25 | GRHLIFCHSKKKCDE | HCV NS3 1393 | 249 |
| 35.0107 | TVDFSLDPTFTIETT | HCV 1466 | 250 |
| 35.0106 | VVVVATDALMTGYTG | HCV 1437 | 251 |

TABLE 4

HCV derived CTL epitopes

| | | | | |
|---|---|---|---|---|
| Supertype | Peptide | Sequence | Source | SEQ ID NO: |

| | | | | |
|---|---|---|---|---|
| A2 | 1090.18 | FLLLADARV | HCV NS I/E2 728 | 252 |
| | 1073.05 | LLFNILGGWV | HCV NS4 1812 | 253 |
| | 1013.02 | YLVAYQATV | HCV NS3 1590 | 254 |
| | 1013.1002 | DLMGYIPLV | HCV Core 132 | 255 |
| | 1090.22 | RLIVFPDLGV | HCV NSS 2611 | 256 |
| | 24.0075 | VLVGGVLAA | HCV NS4 1666 | 257 |
| | 24.0073 | WMNRLIAFA | HCV NS4 1920 | 258 |
| | 1174.08 | HMWNFISGI | HCV NS4 1769 | 259 |
| | 1073.06 | ILAGYGAGV | HCV NS4 1851 | 260 |
| | 24.0071 | LLFLLLADA | HCV NS1/E2 726 | 261 |
| | 1073.07 | YLLPRRGPRL | HCV Core 35 | 262 |
| | 1.0119 | YLVTRHADV | HCV NS3 1136 | 263 |
| A3 | 1.0952 | KTSERSQPR | HCV Core 51 | 264 |
| | 1073.10 | GVAGALVAFK | HCV NS4 1863 | 265 |
| | 1.0123 | LIFCHSKKK | HCV NS3 1391 | 266 |
| | 1.0955 | QLFTFSPRR | HCV E1 290 | 267 |
| | 1073.11 | RLGVRATRK | HCV Core 43 | 268 |
| | 1073.13 | RMYVGGVEHR | HCV NS1/E2 635 | 269 |
| | 24.0090 | VAGALVAFK | HCV NS4 1864 | 270 |
| | F104.01 | VGIYLLPNR | HCV NS5 3036 | 271 |
| B7 | 1145.12 | LPGCSFSIF | HCV Core 168 | 272 |
| | 29.0035 | IPFYGKAI | HCV 1378 | 273 |
| Other | 1069.62 | CTCGSSDLY | HCV NS3 1128 | 274 |
| | 24.0092 | FWAKHMWNF | HCV NS4 1765 | 275 |
| | 13.0019 | LSAFSLHSY | HCV NS5 2922 | 276 |
| A3 | 24.0086 | LGFGAYMSK | HCV NS3 1267 | 277 |
| | 1174.21 | RVCEKMALY | HCV NS5 2621 | 278 |
| | 1174.16 | WMNSTGFTK | HCV NS1/E2 557 | 279 |
| | 1073.04 | TLHGPTPLLY | HCV NS3 1622 | 280 |
| B7 | 16.0012 | FPYLVAYQA | HCV NS3 1588 | 281 |
| | 15.0047 | YPCTVNFTI | HCV NS1/E2 623 | 282 |
| Other | 24.0093 | EVDGVRLHRY | HCV NS5 2129 | 283 |
| | 3.0417 | LTCGFADLMGY | HCV 126 | 284 |
| | 1073.01 | NIVDVQYLY | HCV E1 700 | 285 |
| | 1.0509 | GLSAFSLHSY | HCV NS5 2921 | 286 |
| | 1073.17 | MYVGDLCGSVF | HCV E1 275 | 287 |
| | 1073.18 | MYVGGVEHRL | HCV NS1/E2 633 | 288 |
| | 13.075 | QYLAGLSTL | HCV NS4 1778 | 289 |
| | 1145.13 | FPGCSFSIF | HCV Core 168 | 290 |
| | 1145.25 | LPGCMFSIF | HCV Core 168 | 291 |
| | 1292.24 | LPGCSFSII | HCV Core 169 | 292 |
| | 1145.14 | LPVCSFSIF | HCV Core 168 | 293 |
| | 1145.15 | LPGCSFSYF | HCV Core 168 | 294 |

TABLE 5

HIV derived HTL epitopes

| | | | |
|---|---|---|---|
| Peptide | Sequence | Source | SEQ ID NO: |

| | | | |
|---|---|---|---|
| | GEIYKRWIILGLNKIVRMYSPTSILD | HIV1 GAG 294-319 | 295 |
| | KRWIILGLNKIVRMYSPTSILD | HIV gag 298-319 | 296 |
| 27.0313 | KRWIILGLNKIVRMY | HIV 1 GAG 298 | 297 |
| 27.0311 | GEIYKRWIILGLNKI | HIV 1 GAG 294 | 298 |
| 27.0354 | WEFVNTPPLVKLWYQ | HIV1 POL 596 | 299 |
| 27.0377 | QKQITKIQNFRVYYR | HIV1 POL 956 | 300 |
| | EKVYLAWVPAHKGIGG | HIV1 POL 711-726 | 301 |
| 1280.03 | KVYLAWVPAHKGIGG | HIV POL 712 | 302 |
| 27.0361 | EKVYLAWVPAHKGIG | HIV1 POL 711 | 303 |
| | PIVQNIQGQMVHQAISPRTLNA | HIV1 gag 165-186 | 304 |
| 27.0304 | QGQMVHQAISPRTLN | HIV 1 GAG 171 | 305 |
| 27.0297 | QHLLQLTVWGIKQLQ | HIV1 ENV 729 | 306 |
| 27.0344 | SPAIFQSSMTKILEP | HIV1 POL 335 | 307 |
| F091.15 | IKQFINMWQEVGKAMY | HIV1 ENV 566 | 308 |
| 27.0341 | FRKYTAFTIPSINNE | HIV1 POL 303 | 309 |
| 27.0364 | HSNWRAMASDFNLPP | HIV1 POL 758 | 310 |
| 27.0373 | KTAVQMAVFIHNFKR | HIV 1 POL 915 | 311 |
| | DRVHPVHAGPIAPGQMREPRGS | HIV GAG 245 | 312 |
| | AFSPEVIPMFSALSEGATPQDLNTML HIV gag 195-216 | 313 | |
| | AFSPEVIPMFSALSEGATPQDL | HIV gag 195-216 | 314 |
| 200.06 | SALSEGATPQDLNTML | HIV gag 205 | 315 |
| 27.0307 | SPEVIPMFSALSEGA | HIV gag 197 | 316 |
| | LQEQIGWMTNNPPIPVGEIYKR | HIV gag 275 | 317 |
| 27.0310 | QEQIGWMTNNPPIPV | HIV gag 276 | 318 |
| 35.0135 | YRKILRQRKIDRLID | HIV VPU 31 | 319 |
| 35.0131 | WAGIKQEFGIPYNPQ | HIV POL 874 | 320 |
| 35.0127 | EVNIVTDSQYALGII | HIV POL 674 | 321 |
| 35.0125 | AETFYVDGAANRETK | HIV POL 619 | 322 |
| 35.0133 | GAVVIQDNSDIKVVP | HIV POL 989 | 323 |

TABLE 6
HIV derived CTL epitopes

| | | | | |
|---|---|---|---|---|
| Supertype | Peptide | Sequence | Source | SEQ ID NO: |

| | | | | |
|---|---|---|---|---|
| A2 | 25.0148 | MASDFNLPPV | HIV1 POL 70 | 324 |
| | 1069.32 | VLAEAMSQV | HIV gag 397 | 325 |
| | 1211.04 | KLTPLCVTL | HIV ENV 134 | 326 |
| | 25.0062 | KILVGKLNWA | HIV1 POL 87 | 463 |
| | 25.0039 | LTFGWCFKL | HIV1 NEF 62 | 327 |
| | 941.031 | ILKEPVHGV | HIV1 pol 476-484 | 101 |
| | 25.0035 | MTNNPPIPV | HIV1 GAG 34 | 328 |
| | 25.0057 | RILQQLLFI | HIV1 VPR 72 | 329 |
| A3 | 1.0944 | AVFIHNFKR | HIV POL 1434 | 330 |
| | 1.1056 | KIQNFRVYYR | HIV POL 1474 | 331 |
| | 1069.49 | QMAVFIHNFK | HIV pol 1432 | 332 |
| | 966.0102 | AIFQSSMTK | HIV pol 337 | 333 |
| | 1150.14 | MAVFIHNFK | HIV pol 909 | 334 |
| | 940.03 | QVPLRPMTYK | HIV nef 73-82 | 335 |
| | 25.0175 | TTLFCASDAK | HIV1 ENV 81 | 336 |
| | 1069.43 | TVYYGVPVWK | HIV env 49 | 105 |
| | 25.0209 | VTIKIGGQLK | HIV1 POL 65 | 337 |
| B7 | 1146.01 | FPVRPQVPL | HIV nef 84-92 | 338 |
| | 29.0060 | IPIHYCAPA | HIV env 293 | 339 |
| | 15.0073 | FPISPIETV | HIV POL 171 | 340 |
| | 29.0056 | CPKVSFEPI | HIV env 285 | 341 |
| | 29.0107 | IPYNPQSQGVV | HIV pol 883 | 342 |
| A2 | 25.0151 | CTLNFPISPI | HIV1 POL96 | 343 |
| | 25.0143 | LTPGWCFKLV | HIV1 NEFP 62 | 344 |
| | 25.0043 | YTAFTIPSI | HIV1 POL 83 | 345 |
| | 25.0055 | AIIRILQQL | HIV1 VPR 76 | 346 |
| | 25.0049 | ALVEICTEM | HIV1 POL 52 | 347 |
| | 25.0032 | LLQLTVWGI | HIV1 ENV 61 | 348 |
| | 25.0050 | LVGPTPVNI | HIV1 POL 100 | 349 |
| | 25.0047 | KAACWWAGI | HIV1 POL 65 | 350 |
| | 25.0162 | KMIGGIGGFI | HIV 1 POL 96 | 351 |
| | 25.0052 | RAMASDFNL | HIV1 POL 78 | 352 |
| | 1211.09 | SLLNATDIAV | HIV ENV 814 | 353 |
| A2 | 25.0041 | TLNFPISPI | HIV1 POL 96 | 354 |
| A3 | 1.0046 | IVIWGKTPK | HIV POL 1075 | 355 |
| | 25.0064 | MVHQAISPR | HIV1 GAG 45 | 356 |
| | 1.0062 | YLAWVPAHK | HIV POL 1227 | 357 |
| | 1.0942 | MTKILEPFR | HIV POL 859 | 358 |
| | 25.0184 | QMVHQAISPR | HIV1 GAG 45 | 359 |
| | 1069.48 | AVFIHNFKRK | HIV pol 1434 | 360 |
| | 1069.44 | KLAGRWPVK | HIV pol 1358 | 361 |
| | 1069.42 | KVYLAWVPAHK | HIV pol 1225 | 362 |
| | 1.0024 | NTPVFAIKK | HIV pol 752 | 363 |
| | 25.0062 | RIVELLGRR | HIV1 ENV 53 | 364 |
| | 25.0095 | TIKIGGQLK | HIV1 POL 65 | 365 |
| | 25.0078 | TLFCASDAK | HIV1 ENM 82 | 366 |
| | 25.0104 | VMIVWQVDR | HIV1 VIF 83 | 367 |
| | 1069.47 | VTVYYGVPVWK | HIV env 48 | 368 |
| B7 | 15.0268 | YPLASLRSLF | HIV GAG 507 | 369 |
| | 1292.13 | HPVHAGPIA | HIV GAG 248 | 370 |
| | 19.0044 | VPLQLPPL | HIV con. REV 71 | 371 |
| Other | 1.0431 | EVNIVTDSQY | HIV POL 1187 | 372 |
| | 1.0014 | FRDYVDRFY | HIV GAG 298 | 373 |
| | 25.0113 | IWGCSGKLI | HIV1 ENM 69 | 374 |
| | 25.0127 | IYETYGDTW | HIV1 VPR 92 | 375 |
| | 1069.60 | IYQEPFKNL | HIV pol 1036 | 376 |
| | 2.0129 | IYQYMDDLY | HIV pol 359 | 377 |
| | 25.0128 | PYNEWTLEL | HIV1 VPR 56 | 378 |
| | 25.0123 | PYNTPVFAI | HIV1 POL 74 | 379 |
| | 1069.57 | RYLKDQQLL | HIV env 2778 | 380 |
| | 1069.58 | RYLRDQQLL | HIV env 2778 | 381 |
| | 1069.59 | TYQIYQEPF | HIV pol 1033 | 382 |
| | 1069.27 | VIYQYMDDLY | HIV pol 358 | 383 |
| | 1069.26 | VTVLDVGDAY | HIV pol 265 | 384 |
| | 25.0115 | VWKEATTTL | HIV1 ENV 47 | 385 |
| | 25.0218 | VWYEATITLF | HIV1 ENV 47 | 386 |
| | 25.0219 | YMQATWIPEW | HIV1 POL 96 | 387 |
| A2 | 1211.4 | SLLNATAIAV HIV MN gp 160 814(a) | 388 | |
| A3 | F105.21 | AIFQRSMTR | HIV pol 337(a) | 389 |
| | F105.17 | AIFQSSMTR | HIV pol 337(a) | 390 |
| | F105.02 | GIFQSSMTY | HIV pol 337(a) | 391 |
| | F105.03 | AAFQSSMTK | HIV pol 337(a) | 392 |
| | F105.04 | AIAQSSMTK | HIV pol 337(a) | 393 |
| | F105.05 | AIFASSMTK | HIV pol 337(a) | 394 |
| | F105.06 | AIFQASMTK | HIV pol 337(a) | 395 |
| | F105.07 | AIFQSAMTK | HIV pol 337(a) | 396 |
| | F105.08 | AIFQSSATK | HIV pot 337(a) | 397 |
| | F105.09 | AIFQSSMAK | HIV pol 337(a) | 398 |
| | F105.11 | FIFQSSMTK | HIV pol 337(a) | 399 |
| | F105.12 | SIFQSSMTK | HIV pol 337(a) | 400 |
| | F105.16 | AIFQCSMTK | HIV pol 337(a) | 401 |
| B7 | 1145.03 | FPVRPQFPL | HIV nef 84-92 analog | 402 |
| | 1181.03 | FPVRPQVPI | HIV nef 84-92(a) | 403 |
| | 1292.14 | HPVHAGPII | HIV GAG 248 | 404 |
| | 1292.09 | FPISPIETI | HIV POL 179 | 405 |
| | 1145.02 | FPVFQVPL | HIV nef 84-92 analog | 406 |
| | 1145.22 | TPVRMQVPL | HIV nef 84-92 analog | 407 |
| | 1181.04 | FPVRPQVPM | HIV nef 84-92(a) | 408 |
| | 1181.01 | FPVRPQVPA | HIV nef 84-92(a) | 409 |
| | 1181.02 | FPVRPQVPV | HIV nef 84-92(a) | 410 |
| | 1181.05 | FPVRPQVPF | HIV nef 84-92(a) | 411 |
| | 1181.06 | FPVRPQVPW | HIV nef 84-92(a) | 412 |

TABLE 7

P. falciparum derived HTL epitopes

| | | | |
|---|---|---|---|
| Peptide | Sequence | Source | SEQ ID NO: |

| | | | |
|---|---|---|---|
| F125.04 | RHNWVNHAVPLAMKLI | PfSSP2 61 | 473 |
| 1188.34 | HNWVNHAVPLAMKLI | PfSSP2 62 | 414 |
| 1188.16 | KSKYKLATSVLAGLL | PfEXP1 71 | 415 |
| | LVNLLIFHINGKIIKNSE | PfLSA1 13 | 416 |
| F125.02 | LVNLLIFHINGKIIKNS | PfLSA1 13 | 417 |
| 27.0402 | LLIFHINGKIIKNSE | PfLSA1 16 | 418 |
| 1188.32 | GLAYKFVVPGAATPY | PfSSP2 512 | 419 |
| 27.0392 | SSVFNVVNSSIGLIM | PfCSP 410 | 420 |
| 27.0417 | VKNVIGPFMKAVCVE | PfSSP2 223 | 421 |
| 27.0388 | MRKLAILSVSSFLFV | PfCSP 2 | 422 |
| 27.0387 | MNYYGKQENWYSLKK | PfCSP 53 | 423 |
| 1188.38 | KYKIAGGIAGGLALL | PfSSP2 494 | 424 |
| 1188.13 | AGLLGNVSTVLLGGV | PfEXP1 82 | 425 |
| 27.0408 | QTNFKSLLRNLGVSE | PfLSA1 94 | 426 |
| 35.0171 | PDSIQDSLKESRKLN | PfSSP2 165 | 427 |
| 35.0172 | KCNLYADSAWENVKN | PfSSP2 211 | 428 |

TABLE 8

P. falciparum derived CTL epitopes

| | | | | |
|---|---|---|---|---|
| Supertype | Peptide | Sequence | Source | SEQ ID NO: |

| | | | | |
|---|---|---|---|---|
| A2 | 116721 | FLIFFDLFLV | PfSSP2 14 | 429 |
| | 116708 | GLIMVLSFL | PfCSP 425 | 430 |
| | 1167.12 | VLAGLLGNV | PfEXP1 80 | 431 |
| | 1167.13 | KILSVFFLA | PfEXP1 2 | 432 |
| | 1167.10 | GLLGNVSTV | PfEXP183 | 433 |
| | 1167.18 | ILSVSSFLFV | PfCSP 7 | 434 |
| | 1167.19 | VLLGGVGLVL | PfEXP 191 | 435 |
| A3 | 1167.36 | LACAGLAYK | PfSSP2 511 | 436 |
| | 1167.32 | QTNFKSLLR | PfLSAI 94 | 437 |
| | 1167.43 | VTCGNGIQVR | PfCSP 375 | 438 |
| | 1167.24 | ALFFIIFNK | PfEXP1 10 | 439 |
| | 1167.28 | GVSENIFLK | PfLSA1 105 | 440 |
| | 1167.47 | HVLSHNSYEK | PfLSA1 59 | 441 |
| | 1167.51 | LLACAGLAYK | PfSSP2 510 | 442 |
| | 1167.46 | FILVNLLIFH | PfLSA1 11 | 443 |
| B7 | 1101.03 | MPLETQLAI | PfSHEBA 77 | 444 |
| | 1167.61 | TPYAGEPAPF | PfSSP2 539 | 445 |
| A2 | 1167.14 | FLIFFDLFL | PfSSP2 14 | 446 |
| | 1167.16 | FMKAVCVEV | PfSSP2 230 | 447 |
| | 1167.15 | LIFFDLFLV | PfSSP2 15 | 448 |
| | 1167.17 | LLMDCSGSI | PfSSP2 51 | 449 |
| | 1167.09 | VLLGGVGLV | PfEXP1 91 | 450 |
| B7 | 19.0051 | LPYGRTNL | PfSSP2 126 | 451 |
| Other | 16.0245 | FQDEENIGIY | PfLSA1 1794 | 452 |
| | 16.0040 | FVEALFQEY | PfCSP 15 | 453 |
| | 1167.54 | FYFILVNLL | PfLSA1 9 | 454 |
| | 1167.53 | KYKLATSVL | PfEXP1 73 | 455 |
| | 1167.56 | KYLVIVFLI | PfSSP2 8 | 456 |
| | 15.0184 | LPSENERGY | PfLSA1 1663 | 457 |
| | 16.0130 | PSDGKCNLY | PfSSP2 207 | 458 |
| | 16.0077 | PSENERGYY | PfLSA 1 1664 | 459 |
| | 1167.57 | PYAGEPAPF | PfSSP2 528 | 460 |
| | 1167.55 | YYIPHQSSL | PfLSA1 1671 | 461 |

TABLE 9
Activation of T Cell Proliferation by Expression
Vectors Encoding MHC Class II Epitopes Fused to MHC
Class II Targeting Sequences

Stimulating Peptide¹

| Immunogen | PADRE | OVA 323 | CORE 128 |
|---|---|---|---|
| peptide + CFA² | 3.0 (11) | 2.7 (1.2) | 3.2 (1.4) |
| pEP2.(PAOS).(−) | — | — | — |
| pEP2.(AOS).(−) | 5.6 (1.8) | — | — |
| pEP2.(PAOS).(sigTh) | 5.0 (2.9) | — | 2.6 (1.5) |
| pEP2.(PAOS).(lgαTh) | 5.6 (2.1) | — | 3.0 (1.6) |
| pEP2.(PAOS).(LampTh) | 3.8 (1.7) | — | 3 |
| pEP2.(PAOS).(liTh) | 5.2 (2.0) | 3.2 (1.5) | 3.7(1.5) |
| pEP2.(PAOS).(H2M) | 3.3 (1.3) | — | 2.8 |

¹Geometric mean of cultures with SI ≧ 2.
²Proliferative response measured in the lymph node.

TABLE 10

CTL Epitopes in CDNA Minigene

Immunogenicity In Vivo (IFA)

| | | | | | | |
|---|---|---|---|---|---|---|
| | | | | | CTL | |
| | | | MHC | No. CTL- | Response | |
| | | MHC | Binding | Positive | (GeoMean | |
| Epitope | Sequence | Restrict. | Affinity | Cultures | ×/÷SD)^{b} | SEQ ID NO: |

| | | | | | | |
|---|---|---|---|---|---|---|
| | | | IC30% (nM) | | ΔLU | |
| HBV Core 18 | FLPSDFFPSV | A2.1 | 3 | 6/6 | 73.0 (1.1) | 91 |
| HBV Env 335 | WLSLLVPFV | A2.1 | 5 | 4/6 | 5.3 (1.6) | 107 |
| HBV Pol 455 | GLSRYVARL | A2.1 | 76 | ND^{c} | ND | 99 |
| HIV Env 120 | KLTPLCVTL | A2.1 | 102 | 2/5 | 6.4 (1.3) | 93 |
| HIV Pol 476 | ILKEPVHGV | A2.1 | 192 | 2/5 | 15.2 (2.9) | 101 |
| HBV Pol 55 1-A | YMDDVVLGA | A2.1 | 200 | 0/6 | — | 95 |
| HBV Pol 55 1-V | YMDDVVLGV | A2.1 | 5 | 6/6 | 8.2 (2.3) | 462 |
| HIV Env 49 | TVYYGVPVWK | A11 | 4 | 28/33 | 13.4 (3.1) | 105 |
| HBV Core 141 | STLPETRVVRR | A11 | 4 | 6/6 | 12.1 (2.6) | 103 |
| HBV Pol 149 | HTLWKAGILYK | A11 | 14 | 6/6 | 13.1 (1.2) | 88 |

^{a}Peptide tested in HLA-A2.1/K^{b}H-2^{bxs} transgenic mice by co-immunizing with a T helper cell peptide in IFA.
^{b}Geometric mean CTL response of positive cultures.
^{c}ND, not done.

TABLE 11
Summary of Immunogenicity of pMin.1 DNA
construct in HLA A2.1/K^{b} transgenic mice

CTL Response^{a}

| | No. Positive | Geo. Mean Response Positive |
|---|---|---|
| Epitope | Cultures/Total^{b} | Cultures [×/ ÷ SD] |
| | | ΔLU |
| HBV Core 18 | 9/9 | 455.5 [2.2] |
| HIV Env 120 | 12/12 | 211.9 [3.7] |
| HBV Pol 551-V | 9/9 | 126.1 [2.8] |
| HBV Pol 455 | 12/12 | 738.6 [1.3] |
| HIV Pol 476 | 11/11 | 716.7 [1.5] |
| HBV Env 335 | 12/12 | 43.7 [1.8] |
| HBV Core 18 | 10/10 | 349.3 [1.8] |
| (Theradigm)^{c} | | |

^{a}Mice were immunized with pMin.1 DNA or Theradigm-HBV lipopeptide and CTL activity in splenocyte cultures was determined after in vitro stimulation with individual peptide epitopes. Results from four independent experiments are shown.
^{b}See Example V, Materials and Methods for definition of a CTL-positive culture.
^{c}Response of mice immunized with Theradigm-HBV lipopeptide containing the HBV Core 18 epitope.

TABLE 12
Summary of immunogenicity
in HLA A11/K^{b} transgenic mice

CTL Response^{a}

| | No. Positive | Geo. Mean Response |
|---|---|---|
| Epitope | Cultures/Total^{b} | Positive Cultures [×/ ± SD] |
| | | ΔLU |
| HBV Core 141 | 5/9 | 128.1 [1.6] |
| HBV Pol 149 | 6/9 | 267.1 [2.2] |
| HIV Env 43 | 9/9 | 40.1 [2.9] |

^{a}Mice were immunized with pMin.1 DNA and CTL activity in splenocyte cultures was determined after in vitro stimulation with individual A11-restricted epitopes. The geometric mean CTL response from three independent experiments are shown.
^{b}Definition of a CTL-positive culture is described in Example V, Materials and Methods.

What is claimed:

1. An expression system which comprises a promoter operably linked to a nucleotide sequence which encodes a peptide comprising a first amino acid sequence which is a major histocompatibility (MHC) targeting sequence fused to a second amino acid sequence encoding a Class I MHC restricted CTL peptide epitope and an HTL peptide epitope, wherein the CTL peptide epitope is selected from the group consisting of HCV CTL peptide epitopes set forth as SEQ ID NOs: 252-270, 272, and 274-294, and an analog of each of the foregoing CTL peptide epitopes.

2. The expression system of claim 1, wherein the HTL peptide epitope is a universal HTL peptide epitope.

3. The method of claim 2, wherein the universal HTL peptide epitope is a panDR epitope.

4. The method of claim 3, wherein the panDR epitope comprises the sequence Ala Lys Phe Val Ala Ala Trp Thr Leu Lys Ala Ala Ala (SEQ ID NO:52).

5. The method of claim 1, wherein the HTL peptide epitope is an HCV HTL peptide epitope.

6. The method of claim 5, wherein the HCV HTL peptide epitope is selected from the group consisting of HTL peptide epitopes set forth as SEQ ID NOs: 236-251, and an analog of each of the foregoing HTL peptide epitopes.

7. The method of claim 1, wherein the MHC targeting sequence is the signal sequence from Ig kappa.

8. The method of claim 1, wherein the MHC targeting sequence is the signal sequence from insulin.

9. The method of claim 1, wherein the MHC targeting sequence is the signal sequence from tissue plasminogen.

10. The method of claim 1, wherein the MHC targeting sequence is the signal sequence from LAMP-1.

11. A method to induce an immune response in a subject which comprises administering to a mammalian subject the expression system of claim 1.

12. An expression system which comprises a promoter operably linked to a nucleotide sequence which encodes a peptide comprising a first amino acid sequence which is a MHC targeting sequence fused to a second amino acid sequence encoding a class I MHC restricted CTL peptide epitope and HCV HTL peptide epitope, wherein the HCV HTL peptide epitope is selected from the group consisting of HCV HTL peptide epitopes set forth as SEQ ID NOs: 236-251, and an analog of each of the foregoing HTL peptide epitopes.

13. The expression system of claim 12, wherein the CTL peptide epitope is an HCV CTL peptide epitope.

14. The expression system of claim 13, wherein the CTL peptide epitope is an A2 peptide epitope.

15. The expression system of claim 13, wherein the CTL peptide epitope is an A3 peptide epitope.

16. The expression system of claim 13, wherein the CTL peptide epitope is a B7 peptide epitope.

17. The expression system of claim 13, wherein the CTL peptide epitope is selected from the group consisting of CTL peptide epitopes set forth as SEQ ID NOs: 252-270, 272, and 274-294, and an analog of each of the foregoing CTL peptide epitopes.

18. The method of claim 12, wherein the MHC targeting sequence is the signal sequence from Ig kappa.

19. The method of claim 12, wherein the MHC targeting sequence is the signal sequence from insulin.

20. The method of claim 12, wherein the MHC targeting sequence is the signal sequence from tissue plasminogen.

21. The method of claim 12, wherein the MHC targeting sequence is the signal sequence from LAMP-1.

22. A method to induce an immune response in a subject which comprises administering to a mammalian subject the expression system of claim 12.
